SEQUENCE LISTING (207 sequences)

SEQ ID NO: 1 - protein, 110 aa, artificial sequence
  primer
EQLMESGGDLVKPGASLTLTCTASGFDVSGSYYSHWVRQAPGKGLEWIGMIYNGDGSTWYPSWVNGRFSISKTSSTTVTLQMTSLTAADTATYFCAKNLGLWGPGTPGHR

SEQ ID NO: 2 - protein, 110 aa, artificial sequence
  primer
QSVEESGGDLVKPGASLTLTCTASGFSFSSNYYIWWVRQAPGKGLEWIGWINTVSDGAYYASWAKGRFTISKTSSTTVTLQMTSLTVADTATYFCTNNGLLWGPGTLVTV

SEQ ID NO: 3 - protein, 110 aa, artificial sequence
  primer
EQLMESGGDLVKPGASLTLTCTASGFDVSGSYYSHWVRQAPGKGLEWIGMIYNGDGSTWYPSWVNGRFSISKTSSTTVTLQMTSLTAADTATYFCARDLGLWGPGTLVTV

SEQ ID NO: 4 - protein, 110 aa, artificial sequence; identical to SEQ ID NO: 3
  primer

SEQ ID NO: 5 - protein, 110 aa, artificial sequence; identical to SEQ ID NO: 3
  primer

SEQ ID NO: 6 - protein, 110 aa, artificial sequence
  primer
QSLEESGGDLVKPGASLTLTCTASGFDVSGTYYSHWVRQAPGKGLEWIGMIYNGDGSTWYPSWVNGRFSISKTSSTTVTVQMTSLTAADTATYFCARDLGLWGPGTQVTV

SEQ ID NO: 7 - protein, 96 aa, artificial sequence
  primer
AVGGTVTIKCQSSQSIHNNNYLSWYQQKPGQPPKLLIYQASTLASGVPSRFKGSGSGTQFTLTISDLECDDAATYYCAGGYDCSSADCFAFGGGTE

SEQ ID NO: 8 - protein, 95 aa, artificial sequence
  primer
PVGDTVTINCQSSQNVYSNYLSWFQKKPGQPPKLLIYRASNLASGVPPRFSGSGSGTDYTLTIGGVQAEDAATYYCLGGYSYSSTGLTFGAGTNV

SEQ ID NO: 9 - protein, 94 aa, artificial sequence
  primer
AVGGTVTIKCQSSQSINNYLSWYQQKPGQPPKLLIYQASTLASGVPSRFKGSGSGTQFTLTISDLECDDAATYYCAGGYDCSSADCFAFGGGTE

SEQ ID NO: 10 - protein, 96 aa, artificial sequence
  primer
AVGGTVTIKCQSSQSIVYNNYLSWYQQKPGQPPKLLIYQASTLASGVPSRFKGSGSGTQFTLTISDLECDDAATYYCAGGYDCSSADCFAFGGGTE

SEQ ID NO: 11 - protein, 96 aa, artificial sequence; identical to SEQ ID NO: 7
  primer

SEQ ID NO: 12 - protein, 94 aa, artificial sequence
  primer
  misc_feature (18)..(18): Xaa can be any naturally occurring amino acid
AVGGTVTIKCQASQSISXYLAWYQQKPGQPPKLLIYSASKLASGVPSRFRGSGSGTQFTLTISGVQCDDAATYYCAGGYYGGIFAFGGGTELEI

SEQ ID NO: 13 - protein, 20 aa, artificial sequence
  primer
SRLSKVAPVIKARMMEYGGT

SEQ ID NO: 14 - protein, 20 aa, artificial sequence
  primer
NMYAMLIARYKMFPEVKEKG

SEQ ID NO: 15 - protein, 19 aa, artificial sequence
  primer
ALGIGTDSVILIKCDERGK

SEQ ID NO: 16 - protein, 9 aa, artificial sequence
  primer
KGTDVQAWI

SEQ ID NO: 17 - protein, 111 aa, artificial sequence
  primer
EVQLQQSGAELVKPGASVKLSCKASGYTFTSYWMHWVKQRPGHGLEWIGEISPTNGRTNYNEKFKTKATLTVDKSSSTAYMQLSSLTSEDSAVYYCTRLGGLNWGQGTLVT

SEQ ID NO: 18 - protein, 120 aa, artificial sequence
  primer
EVQLQQSGAELVRPGASVTLSCKASGYTFIDYEMHWVKQTPVHGLEWIGTIDPETGGTAYNQKFKDKATLTADKSSSTAYMELRGLTSEDSAVYYCTRWYYDSSYVLYGMDYWGQGTSVT

SEQ ID NO: 19 - protein, 116 aa, artificial sequence
  primer
EVQLQQSGAELVKPGASVKLSCKASGYTFTSFWMHWVKQRPGQGLEWIGEINPSNGRTNYNEKFKSKATLTVDKSSSTAYMQLSSLTSEDSAVYYCARSTMISTGFPYWGQGTLVT

SEQ ID NO: 20 - protein, 111 aa, artificial sequence; identical to SEQ ID NO: 17
  primer

SEQ ID NO: 21 - protein, 111 aa, artificial sequence
  primer
EVQLQQPGAELVKPGASVKLSCKASGYTFTSYWMHWVKQRPGHGLEWIGEISPTNGRTNYNEKFKTKATLTVDKSSSTAYMQLSSLTSEDSAVYYCTRLGGLNWGQGTLVT

SEQ ID NO: 22 - protein, 115 aa, artificial sequence
  primer
EVKLVESGGGSVKPGGSLKLSCAASGFTFSNYLMSWVRQTPEKRLEWVATISSGGGFTYYPDNVKGRFTISRDNAKNTLYLQMSSLKSEDTAMYYCSREFTTKAMDYWGQGTSVT

SEQ ID NO: 23 - protein, 117 aa, artificial sequence
  primer
EVQLVESGGGLVKPGGSLKLSCAASGFTFSSYTMSWVRQTPEKRLEWVATISSGGNYTFYPDSVKGRFTISRDNAKNTLYLQMSSLKSEDTAMYYCTRGLFYGYAWFNYWGQGTLVT

SEQ ID NO: 24 - protein, 34 aa, artificial sequence
  primer
  misc_feature (3)..(3): Xaa can be any naturally occurring amino acid
  misc_feature (9)..(9): Xaa can be any naturally occurring amino acid
  misc_feature (20)..(20): Xaa can be any naturally occurring amino acid
  misc_feature (26)..(26): Xaa can be any naturally occurring amino acid
  misc_feature (31)..(31): Xaa can be any naturally occurring amino acid
EVXLQQSGXELVRTGASVKXSCKASXYSFTXDYI

SEQ ID NO: 25 - protein, 111 aa, artificial sequence; identical to SEQ ID NO: 21
  primer

SEQ ID NO: 26 - protein, 115 aa, artificial sequence; identical to SEQ ID NO: 22
  primer

SEQ ID NO: 27 - protein, 108 aa, artificial sequence
  primer
  misc_feature (10)..(10): Xaa can be any naturally occurring amino acid
  misc_feature (13)..(13): Xaa can be any naturally occurring amino acid
  misc_feature (59)..(59): Xaa can be any naturally occurring amino acid
  misc_feature (62)..(62): Xaa can be any naturally occurring amino acid
  misc_feature (66)..(66): Xaa can be any naturally occurring amino acid
  misc_feature (99)..(99): Xaa can be any naturally occurring amino acid
  misc_feature (103)..(103): Xaa can be any naturally occurring amino acid
  misc_feature (106)..(106): Xaa can be any naturally occurring amino acid
  misc_feature (108)..(108): Xaa can be any naturally occurring amino acid
EVQLQQSGPXLVXPGASVKMSCKASGYTFTGYWIHWVKQRPGQGLEWIGMIDPSNSETXLNXKFKXKATLNVDNSSNTAYMHLSSLTSEDSAVYYCARXYGDXYAXFX

SEQ ID NO: 28 - protein, 115 aa, artificial sequence
  primer
EVKLVESGGGSVKPGGSLKLSCAASGFTFSKYLMSWVRQTPEKRLEWVATISSGGSFTYYPDNVKGRFTISRDNAKNTLYLQMSSLKSEDTAMYYCTREFTTKAMDYWGQGTSVT

SEQ ID NO: 29 - protein, 117 aa, artificial sequence; identical to SEQ ID NO: 23
  primer

SEQ ID NO: 30 - protein, 111 aa, artificial sequence; identical to SEQ ID NO: 21
  primer

SEQ ID NO: 31 - protein, 116 aa, artificial sequence
  primer
EVQLQQSGAEPARPGASVKLSCKASGYTFTDYYIHWVKQRTGQGLEWIGEIYPGTDNTYYNEKFKGKATLTADKSSSTAYMQLSSLTSEDSAVYFCARQLGHYFAMDYWGQGTSVT

SEQ ID NO: 32 - protein, 111 aa, artificial sequence; identical to SEQ ID NO: 17
  primer

SEQ ID NO: 33 - protein, 117 aa, artificial sequence
  primer
EVKLVESGGGLVQPGGSLKLSCAASGFTFSSYTMSWVRQTPEKRLEWVATISSGGNYTFYPDSVKGRFTISRDNVKNTLYLQMSSLKSEDTAMYYCTRGLFYGYAWFNYWGQGTLVT

SEQ ID NO: 34 - protein, 111 aa, artificial sequence; identical to SEQ ID NO: 21
  primer

SEQ ID NO: 35 - protein, 115 aa, artificial sequence; identical to SEQ ID NO: 28
  primer

SEQ ID NO: 36 - protein, 111 aa, artificial sequence
  primer
EVQLQQPGAELVKPGASVKLSCKASGYTFTSYWMHWVKQRPGHGLEWIGEISPTNGRTNYNEKFKTKATLTVDKSSSTAYMQLSSLTSEDSAVYYCTRLGGLNWGQGTLGT

SEQ ID NO: 37 - protein, 109 aa, artificial sequence
  primer
DIVLTQSPKFMSTSVGDRVSITCKASQSVGTAVAWYQQKPGQSPKLLIYSASNRETGVPDRFTGSGSGTDFTLTISNVQSEDLADYFCQQYSSYPLTFGAGTKLELKRA

SEQ ID NO: 38 - protein, 115 aa, artificial sequence
  primer
DIVMTQSHKFMSTSVGDRVSITCKASQDVGTAVAWYQQKPGQSPKLLIYASTRHTGVPDRFTGSGSGTDFTLTISNVQSEDLADYFCQQYSSYPLTFGAGTKLELKRADAAPTVS

SEQ ID NO: 39 - protein, 108 aa, artificial sequence
  primer
ENVLTQSPAIMSASPGEKVTMTCSASSSVSYMYWYQQKPGSSPRLLIYDTSNLASGVPVRFSGSGSGTSYSLTISRMEAEDAATYYCQQWSSYPPTFGGGTKLEIKRA

SEQ ID NO: 40 - protein, 109 aa, artificial sequence
  primer
DIVLTQSPASLAVSLGQRATISCRASESVDSYGNSFMHWYQQKPGQPPKLLIYRASNLESGIPARFSGSGSRTDFTLTINPVEADDVATYYCQQSNEDPRTFGGGTKLE

SEQ ID NO: 41 - protein, 109 aa, artificial sequence
  primer
DIAVTQSPASLAVSLGQRATISCRASESVDSYGNSFMHWYQQKPGQPPKLLIYLASNLESGVPARFSGSGSRTDFTLTIDPVEADDAATYYCQQNNEDPYTFGGGTKLE

SEQ ID NO: 42 - protein, 109 aa, artificial sequence
  primer
DVVMTQTQKFMSTSVGDRVSITCKASQNVGTAVAWYQQKPGQSPKLLIYSASNRYTGVPDRFTGSGSGTDFTLTISNMQSEDLADYFCQQYSSYPLTFGAGTKLELKRA

SEQ ID NO: 43 - protein, 109 aa, artificial sequence; identical to SEQ ID NO: 42
  primer

SEQ ID NO: 44 - protein, 108 aa, artificial sequence
  primer
DNVLTQSPASLAVSLGQRATISCRASESVDSYGNSFMHWYQKPGQPPKLLIYLASNLESGVPARFSGSGSRTDFTLTIDPVEADDAATYYCLQNNEDPYTFGGGTKLE

SEQ ID NO: 45 - protein, 108 aa, artificial sequence
  primer
DNVLTSHKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLELKRA

SEQ ID NO: 46 - protein, 108 aa, artificial sequence
  primer
DIVMTQSQKFMSTSVGDRVSITCASQNVGTAVAWYQQKPGQSPKLLIYSASNRYTGVPDRFTGSGSGTDFTLTISNMQSEDLADYFCQQYSSYPLTFGAGTKLELKRA

SEQ ID NO: 47 - protein, 108 aa, artificial sequence; identical to SEQ ID NO: 44
  primer

SEQ ID NO: 48 - protein, 109 aa, artificial sequence
  primer
DIVLTQSQKFMSTSVGDRVSITCKASQNVGTAVAWYQQKPGQSPKLLIYSASNRYTGVPDRFTGSGSGTDFTLTISNMQSEDLADYFCQQYSSYPLTFGAGTKLELKRA

SEQ ID NO: 49 - protein, 109 aa, artificial sequence
  primer
DIVLTQSPASLAVSLGQRATISCRASESVDSYGNSFMHWYQQKPGQPPKLLIYLASNLEFGVPARFSGSGSRTDFTLTIDPVEADDAATYYCQQNNEDPYTFGGGTKLE

SEQ ID NO: 50 - protein, 108 aa, artificial sequence; identical to SEQ ID NO: 45
  primer

SEQ ID NO: 51 - protein, 108 aa, artificial sequence
  primer
DVAMTQTQKFMSTSVGDRVSITCKASQNVGTAVAWYQQKPGQSPKLLIYSASNRYTGVPDRFTGSGSGTDFTLTISNMQSEDLADYFCQQYSSYPLTFGAGTKLELKR

SEQ ID NO: 52 - protein, 108 aa, artificial sequence
  primer
DIVMTQSHKFMSTSVGDRVSITCKASQDVGTAVAWYQQKPGQSPKLLIYASTRHTGVPDRFTGSGSGTDFTLTISNVQSEDLADYFCQQYSSYPLTFGAGTKLELKRA

SEQ ID NO: 53 - protein, 105 aa, artificial sequence
  primer
DVVMTQTHKFMSTSVGDRVSITCKASQDVGTAVAWYQQKPGQSPKLLIYWASTRHTGVPDRFTGSGSGTDFTLTISNVQSEDLADYFCQQYSSYPYTFGGGTKLE

SEQ ID NO: 54 - protein, 109 aa, artificial sequence; identical to SEQ ID NO: 42
  primer

SEQ ID NO: 55 - protein, 109 aa, artificial sequence
  primer
DIALTQSPASLAVSLGQRATISCRASESVDSYGNSFMHWYQQKPGQPPKLLIYLASNLESGVPARFSGSGSRTDFTLTIDPVEADDAATYYCQQNNEDPYTFGGGTKLE

SEQ ID NO: 56 - protein, 4 aa, artificial sequence
  primer
LGGL

SEQ ID NO: 57 - protein, 8 aa, artificial sequence
  primer
EFTTKAMD

SEQ ID NO: 58 - protein, 10 aa, artificial sequence
  primer
GLFYGYAWFN

SEQ ID NO: 59 - protein, 9 aa, artificial sequence
  primer
QQYSSYPLT

SEQ ID NO: 60 - protein, 9 aa, artificial sequence
  primer
QQSNEDPRT

SEQ ID NO: 61 - protein, 9 aa, artificial sequence
  primer
QQNNEDPYT

SEQ ID NO: 62 - protein, 9 aa, artificial sequence
  primer
LQNNEDPYT

SEQ ID NO: 63 - protein, 119 aa, artificial sequence
  primer
EVQLQQSGPELEKPGASVKISCKASGYSFTGYNMNWVKQSNGKSLEWIGNIDPYYGDITYNQKFKGKATLTVDKSSSTAYMQLKSLTSEDSAVYYCARTATALYTMDYWGQGTSVTVSS

SEQ ID NO: 64 - protein, 119 aa, artificial sequence; identical to SEQ ID NO: 63
  primer

SEQ ID NO: 65 - protein, 119 aa, artificial sequence; identical to SEQ ID NO: 63
  primer

SEQ ID NO: 66 - protein, 119 aa, artificial sequence
  primer
EVQLQQSGPELEKPGASVKISCKASGYSFTGYNMNWVKQSSGKSLEWIGNFDPYYGVITYNQKFKGKATLTVDKSSSTAYMQLKSLTSEDSAVYYCARTATALYTMDYWGQGTSVTVSS

SEQ ID NO: 67 - protein, 119 aa, artificial sequence; identical to SEQ ID NO: 66
  primer

SEQ ID NO: 68 - protein, 120 aa, artificial sequence
  primer
EVQLQQSGPEVVRPGSSVKMSCKASGYTFTTYWIHWVKQRPGQGLEWIGMIDPSNSEARLNQRFKDKATLNLDQSSNTAYMRLSSLTSEDSAVYYCAGPSDNSYAWFAYWGQGTLVTVSA

SEQ ID NO: 69 - protein, 118 aa, artificial sequence
  primer
EVKLVESGGGLVKPGGSLKLSCAASGFTFNNYAMSWVRQTPEKRLEWVATISSGGSFTFYPDSVKGRFTISRDNAKNTLYLQMTSLRSEDTAMYYCARQATTTAFDYWGQGTTLTVSS

SEQ ID NO: 70 - protein, 119 aa, artificial sequence; identical to SEQ ID NO: 63
  primer

SEQ ID NO: 71 - protein, 119 aa, artificial sequence
  primer
EVQLQQSGPELEKPGASVKISCKASGYSFTGYNMNWVKQSNGKSLEWIGNIDPYYGGSSYNQKFKGKATLTVDKSSSTAYMQLKSLTSEDSAVYYCARTATALYTMDYWGQGTSVTVSS

SEQ ID NO: 72 - protein, 119 aa, artificial sequence
  primer
EVQLQQSGPELEKPGASVKISCKASGYSFTGYNMNWVKQSNGKSLEWIGNIDPYYGGTSYNQKFKGKATLTVDKSSSTAYMQLKSLTSEDSAVYYCARTATALSTMDYWGQGTSVTVSS

SEQ ID NO: 73 - protein, 119 aa, artificial sequence; identical to SEQ ID NO: 71
  primer

SEQ ID NO: 74 - protein, 119 aa, artificial sequence; identical to SEQ ID NO: 71
  primer

SEQ ID NO: 75 - protein, 119 aa, artificial sequence; identical to SEQ ID NO: 63
  primer

SEQ ID NO: 76 - protein, 119 aa, artificial sequence; identical to SEQ ID NO: 71
  primer

SEQ ID NO: 77 - protein, 119 aa, artificial sequence; identical to SEQ ID NO: 63
  primer

SEQ ID NO: 78 - protein, 119 aa, artificial sequence
  primer
EVQLKQSGPELEKPGASVKISCKASGYSFTGYNMNWVKQSNGKSLEWIGNIDPYYGDITYNQKFKGKATLTVDKSSSTAYMQLKSLTSEDSAVYYCARTATALYTMDYWGQGTSVTVSS

SEQ ID NO: 79 - protein, 117 aa, artificial sequence
  primer
EVQLKQSGPGLVQPSQSLSITCTVSGFSLTTYGLHWVRQSPGKGLEWLGVIWRGGNTDYNAAFMSRLSITKDNSKSQVFFKMNSLQADDTAIYYCAKNDYYWGFGDWGQGTLVTVSA

SEQ ID NO: 80 - protein, 119 aa, artificial sequence; identical to SEQ ID NO: 63
  primer

SEQ ID NO: 81 - protein, 119 aa, artificial sequence; identical to SEQ ID NO: 72
  primer

SEQ ID NO: 82 - protein, 113 aa, artificial sequence
  primer
ESGGGSVKPGGSLKLSCAASGFTFSSYLMSWVRQTPEKRLEWVATISSGGSFTYYPDNVKGRFTISRDNAKNTLYLQMSSLKSEDTAMYYCTREFTTKALDYWGQGTSVTVSA

SEQ ID NO: 83 - protein, 10 aa, artificial sequence
  primer
PSDNSYAWFA

SEQ ID NO: 84 - protein, 8 aa, artificial sequence
  primer
QATTTAFD

SEQ ID NO: 85 - protein, 9 aa, artificial sequence
  primer
TATALSTMD

SEQ ID NO: 86 - protein, 8 aa, artificial sequence
  primer
NDYYWGFG

SEQ ID NO: 87 - protein, 9 aa, artificial sequence
  primer
TATALYTMD

SEQ ID NO: 88 - protein, 8 aa, artificial sequence
  primer
EFTTKALD

SEQ ID NO: 89 - protein, 17 aa, artificial sequence
  primer
MIDPSNSEARLNQRFKD

SEQ ID NO: 90 - protein, 17 aa, artificial sequence
  primer
TISSGGSFTFYPDSVKG

SEQ ID NO: 91 - protein, 17 aa, artificial sequence
  primer
NIDPYYGGTSYNQKFKG

SEQ ID NO: 92 - protein, 16 aa, artificial sequence
  primer
VIWRGGNTDYNAAFMS

SEQ ID NO: 93 - protein, 17 aa, artificial sequence
  primer
NFDPYYGVITYNQKFKG

SEQ ID NO: 94 - protein, 17 aa, artificial sequence
  primer
NIDPYYGGSSYNQKFKG

SEQ ID NO: 95 - protein, 17 aa, artificial sequence
  primer
TISSGGSFTYYPDNVKG

SEQ ID NO: 96 - protein, 110 aa, artificial sequence
  primer
EIVLTQSPAIMSASLGERVTMTCTASSSVSSSYLHWYQQKPGSSPKLWIYSTSNLASGVPARFSGSGSGTSYSLTISSMEAEDAATYYCHQYHRSPQTFGGGTKLEIKRA

SEQ ID NO: 97 - protein, 110 aa, artificial sequence
  primer
EIVLTQSPAIMSASLGERVTMTCTASSSVSSSYLHWYQQKPGSSPKLWIYSTSNLASGVPARFSGSGSGNSYSLTISSMEGEDAATYYCQQFTSSPSTFGAGTKLELKRA

SEQ ID NO: 98 - protein, 110 aa, artificial sequence
  primer
QIVLTQSPAIMSASLGERVTMTCTASSSISSSYLHWYQKPGSSPKLWIYSTSNLASGVPARFSGSGSGTSYSLTISSMEAEDAATYYCHQYHRSPPMTFGGGTKLEIKRA

SEQ ID NO: 99 - protein, 110 aa, artificial sequence
  primer
EIVLTQSPAIMSASPGEKVTMTCRASSSVSSSYLHWYQQKSGASPKLWIYSTSNLASGVPARFSGSGSGTSYSLTISSVEAEDAATYYCQQYSGYPLTFGSGTKLEIKRA

SEQ ID NO: 100 - protein, 110 aa, artificial sequence; identical to SEQ ID NO: 99
  primer

SEQ ID NO: 101 - protein, 107 aa, artificial sequence
  primer
QIVLTQSPAIMSASPGEKVTITCSASSSVSYMHWFQKPGTSPKLWIYSTSNLASGVPARFSGSGSGTSYSLTISRMEAEDAATYYCQQRSSYPFTFGSGTKLEMKRA

SEQ ID NO: 102 - protein, 113 aa, artificial sequence
  primer
DIVLTQSPAPLAVSLGQRATISCRASESVDSYGNSFMHWYQQKPGQPPKLLIYLASNLESGVPARFSGSGSRTDFTLTIDPVEADDAATYYCQQNNEDPPTFGGGTKLEIKRA

SEQ ID NO: 103 - protein, 111 aa, artificial sequence
  primer
  misc_feature (7)..(7): Xaa can be any naturally occurring amino acid
EKLCSPXSPAIMSASLGERVTMTCTASSSVSSSYLHWYQQKPGSSPKLWIYSTSNQAPGVPARFSGSGSGNSYSLTISSMEGEDAATYYCQQFTSSPSTFGAGTKLELKRA

SEQ ID NO: 104 - protein, 110 aa, artificial sequence
  primer
EIVLTQSPAIMSASPGEKVTMTCRASSSVSSSYLHWYQQKSGASPKLWIYSTSNLASGVPARFSGSGSGTSYSLTISSVEAEDAATYYCQQYSGYPGTFGGGTKLEIKRA

SEQ ID NO: 105 - protein, 111 aa, artificial sequence
  primer
VLTQSPASLAVSLGQRATISCRASESVDSYGNSFMHWYQQKPGQPPKLLIYLASNLESGVPARFSGSGSRTDFTLTIDPVEADDAATYYCQQNNEDPYTFGGGTKLEIKRA

SEQ ID NO: 106 - protein, 111 aa, artificial sequence
  primer
DKLFSPRSPAIMSASLGERVTMTCTASSSVSSSYLHWYQQKPGSSPKLWIYSTSNLASGVPARFSGSGSGTSYSLTISSMEAEDAATYYCHQYHRSPQTFGGGTKLEIKRA

SEQ ID NO: 107 - protein, 111 aa, artificial sequence
  primer
  misc_feature (6)..(6): Xaa can be any naturally occurring amino acid
ETLCCXRSPAIMSASPGEKVTMTCRASSSVSSSYLHWYQQKSGASPKLWIYSTSNLASGVPARFSGSGSGTSYSLTISSVEAEDAATYYCQQYSGYSLTFGSGTKLEIKRA

SEQ ID NO: 108 - protein, 113 aa, artificial sequence
  primer
DKLFSPRSPAIMSASPGEKVTMTCRASSSVSSSYLHWYQQKSGASPKLWIYSTSNLASGVPARFSGSGSGTSYSLTISSVEAEDAATYYCQQYSGYPLMLTFGAGTKLELKRA

SEQ ID NO: 109 - protein, 102 aa, artificial sequence
  primer
AIMSASPGEKVTMTCRASSSVSSSYLHWYQQKSGASPKLWIYSTSNLASGVPARFSGSGSGTSYSLTISSVEAEDAATYYCQQYGGYPLTFGAGTKLELKRA

SEQ ID NO: 110 - protein, 110 aa, artificial sequence
  primer
  misc_feature (5)..(5): Xaa can be any naturally occurring amino acid
EIVLXQSPAIMSASPGEKVTMTCRASSSVSSSYLHWYQQKSGASPKLWIYSTSNLASGVPARFSGSGSGTSYSLTISSVEAEDAATYYCQQYSGYPGTFGGGTKLEIKRA

SEQ ID NO: 111 - protein, 111 aa, artificial sequence
  primer
EKLCSPRSPAIMSASLGERVTMTCTASSSVSSSYLHWYQQKPGSSPKLWIYSTSNLASGVPARFSGSGSGNSYSLTISSMEGEDAATYYCQQFTSSPSTFGAGTKLELKRA

SEQ ID NO: 112 - protein, 111 aa, artificial sequence
  primer
  misc_feature (6)..(6): Xaa can be any naturally occurring amino acid
EKLCSXRSPAIMSASPGEKVTMTCRASSSVSSSYLHWYQQKSGASPKLWIYSTSNLASGVPARFSGSGSGTSYSLTISSVEAEDAATYYCQQYSGSPLTFGAGTKLELKRA

SEQ ID NO: 113 - protein, 109 aa, artificial sequence
  primer
  misc_feature (6)..(6): Xaa can be any naturally occurring amino acid
EMLCCXRLTNSCPHQETGFSITCKASQDVGTAVAWYQQKPGQSPKLLIYWASTRHTGVPDRFTGSGSGTDFTLTISNVQSEDLADYFCQQYSSYPFTFGSGTKLEIKRA

SEQ ID NO: 114 - protein, 110 aa, artificial sequence; identical to SEQ ID NO: 96
  primer

SEQ ID NO: 115 - protein, 111 aa, artificial sequence
  primer
DKLFSSRSPAIMSASLGERVTMTCTASSSVSSSYLHWYQQKPGSSPKLWIYSTSNLASGVPARFSGSGSGTSYSLTISSMEAEDAATYYCHQYHRSPQTFGGGTKLEIKRA

SEQ ID NO: 116 - protein, 8 aa, artificial sequence
  primer
QYHRSPQT

SEQ ID NO: 117 - protein, 9 aa, artificial sequence
  primer
CQQFTSSPS

SEQ ID NO: 118 - protein, 9 aa, artificial sequence
  primer
QQYSGYPLT

SEQ ID NO: 119 - protein, 9 aa, artificial sequence
  primer
QQYSGYPGT

SEQ ID NO: 120 - protein, 10 aa, artificial sequence
  primer
HQYHRSPPMT

SEQ ID NO: 121 - protein, 9 aa, artificial sequence
  primer
QQRSSYPFT

SEQ ID NO: 122 - protein, 9 aa, artificial sequence
  primer
QQYSSYPFT

SEQ ID NO: 123 - protein, 9 aa, artificial sequence
  primer
QQNNEDPPT

SEQ ID NO: 124 - protein, 9 aa, artificial sequence
  primer
QQYSGYSLT

SEQ ID NO: 125 - protein, 11 aa, artificial sequence
  primer
QQYSGYPLMLT

SEQ ID NO: 126 - protein, 9 aa, artificial sequence
  primer
QQYGGYPLT

SEQ ID NO: 127 - protein, 9 aa, artificial sequence
  primer
QQNNEDPYT

SEQ ID NO: 128 - protein, 9 aa, artificial sequence
  primer
QQYSGSPLT

SEQ ID NO: 129 - protein, 8 aa, artificial sequence
  primer
STSNLASG

SEQ ID NO: 130 - protein, 8 aa, artificial sequence
  primer
LASNLESG

SEQ ID NO: 131 - protein, 8 aa, artificial sequence
  primer
STSNQAPG

SEQ ID NO: 132 - protein, 8 aa, artificial sequence
  primer
WASTRHTG

SEQ ID NO: 133 - protein, 119 aa, artificial sequence
  primer
EVKLVETGGGSVKPGGSLKLSCAASGFTFSSYLMSWVRQTPEKRLEWVATISSGGSFTYYPDNVKGRFTISRDNAKNTLYLQMSSLKSEDTAMYYCTREFTTKALDYWGQGTSVTVSAA

SEQ ID NO: 134 - protein, 119 aa, artificial sequence
  primer
EVKLVESGGGSVKPGGSLKLSCAASGFTFSKYLMSWVRQTPEKRLEWVATISSGGSFTYYPDNVKGRFTISRDNAKNTLYLQMSSLKSEDTAMYYCTREFTTKAMDYWGQGTSVTVSSA

SEQ ID NO: 135 - protein, 120 aa, artificial sequence
  primer
EVQLKQSGPELEKPGASVKISCKASGYSFTGYNMNWVKQSNGKSLEWIGNIDPYYDSISYNQKFKGKATLTVDKSSSTAYMQLKSLTSEDSAVYYCARTATALYTMDYWGQGTSVTVSSA

SEQ ID NO: 136 - protein, 120 aa, artificial sequence
  primer
EVQLQQSGPELEKPGASVKISCKASGYSFTGYNMNWVKQSSGKSLEWIGNFDPYYGVITYNQKFKGKATLTVDKSSSTAYMQLKSLTSEDSAVYYCARTATALYTMDYWGQGTSVTVSSA

SEQ ID NO: 137 - protein, 119 aa, artificial sequence
  primer
EVQLVESGGGSVKPGGSLKLSCAASGFTFSSYLMSWVRQTPEKRLEWVATISSGGSYTYYPDNVKGRFTISRDNAKNTLYLQMSSLKSEDTAMYYCTREFTTKAMDYWGQGTSVTVSSA

SEQ ID NO: 138 - protein, 120 aa, artificial sequence
  primer
EVQLQQSGPALEKPGASVKISCKASGYSFTGYNMNWVKQSSGKSLEWIGNFDPYYGVISYNQKFKGKATLTVDKSSSTAYMQLKSLTSEDSAVYYCARTATALYTMDYWGQGTSVTVSSA

SEQ ID NO: 139 - protein, 119 aa, artificial sequence
  primer
EVKLVESGGGSVKPGGSLKLSCAASGFTFSSYLMSWVRQTPEKRLEWVATISSGGSFTYYPDNVKGRFTISRDNAKNTLYLQMSSLKSEDTAMYYCTREFTTKALDYWGQGTSVTVSAA

SEQ ID NO: 140 - protein, 119 aa, artificial sequence; identical to SEQ ID NO: 139
  primer

SEQ ID NO: 141 - protein, 98 aa, artificial sequence
  primer
  misc_feature (3)..(3): Xaa can be any naturally occurring amino acid
  misc_feature (11)..(12): Xaa can be any naturally occurring amino acid
  misc_feature (28)..(28): Xaa can be any naturally occurring amino acid
  misc_feature (31)..(31): Xaa can be any naturally occurring amino acid
  misc_feature (35)..(35): Xaa can be any naturally occurring amino acid
  misc_feature (38)..(38): Xaa can be any naturally occurring amino acid
  misc_feature (48)..(48): Xaa can be any naturally occurring amino acid
  misc_feature (50)..(50): Xaa can be any naturally occurring amino acid
  misc_feature (52)..(52): Xaa can be any naturally occurring amino acid
  misc_feature (58)..(58): Xaa can be any naturally occurring amino acid
  misc_feature (71)..(71): Xaa can be any naturally occurring amino acid
  misc_feature (75)..(75): Xaa can be any naturally occurring amino acid
EVXLVESGGGXXKPGGSLKISCAASGFXFTXYLMXWVXQTPEKRLEWXAXFXTDWFYXTIQTMRADSPSLXTMPXTPCTCKAASLRTLPCITVQESLR

SEQ ID NO: 142 - protein, 119 aa, artificial sequence
  primer
  misc_feature (31)..(31): Xaa can be any naturally occurring amino acid
  misc_feature (38)..(38): Xaa can be any naturally occurring amino acid
  misc_feature (42)..(42): Xaa can be any naturally occurring amino acid
  misc_feature (56)..(56): Xaa can be any naturally occurring amino acid
  misc_feature (76)..(76): Xaa can be any naturally occurring amino acid
  misc_feature (103)..(104): Xaa can be any naturally occurring amino acid
  misc_feature (114)..(114): Xaa can be any naturally occurring amino acid
  misc_feature (119)..(119): Xaa can be any naturally occurring amino acid
EVKLVESGGGSVKPGGSLKLSCAASGFTFSXYLMSWVXQTPXKRLEWVATISSGGXYTYYPDNVKGRFTISRDNAXNTLYLQMSSLKSEDTAMYYCTREFTTXXMDYWGQGTSXTVSSX

SEQ ID NO: 143 - protein, 120 aa, artificial sequence
  primer
EVQLQQSGPELEKPGASVKISCKASGYSFTGYNMNWVKQSNGKSLEWIGNIDPYYDSISYNQKFKGKATLTVDKSSSTAYMQLKSLTSEDSAVYYCARTATALYTMDYWGQGTSVTVSSA

SEQ ID NO: 144 - protein, 120 aa, artificial sequence
  primer
EVQLQQSGPELEKPGASVKISCKASGYSFTGYNMNWVKQSNGKSLEWIGNIDPYYGSISYNQKFKGKATLTVDKSSSTAYMQLKSLTSEDSAVYYCARTATALYTMDYWGQGTSVTVSSA

SEQ ID NO: 145 - protein, 120 aa, artificial sequence; identical to SEQ ID NO: 136
  primer

SEQ ID NO: 146 - protein, 119 aa, artificial sequence
  primer
EVKLVESGGGSVKPGGSLKLSCAASGFTFSKHLMSWVRQTPEKRLEWVATISSGGSFTYYPDNVKGRFTISRDNAKNTLYLQMSSLKSEDTAMYYCTREFTTKAMDYWGQGTSVTVSSA

SEQ ID NO: 147 - protein, 119 aa, artificial sequence
  primer
EVKLVESGGGSVKPGGSLKLSCAASGFTFSSYLMSWVRQTPEKRLEWVATISSGGSYTYYPDNVKGRFTISRDNAKNTLYLQMSSLKSEDTAMYYCTREFTTKAMDYWGQGTSVTVSSA

SEQ ID NO: 148 - protein, 119 aa, artificial sequence; identical to SEQ ID NO: 134
  primer

SEQ ID NO: 149 - protein, 119 aa, artificial sequence
  primer
EVKLVESGGGSVKPGGSLKLSCAASGFTFSNYLMSWVRQTPEKRLEWVATISSGGGFTYYPDNVKGRFTISRDNAKNTLYLQMSSLKSEDTAMYYCSREFTTKAMDYWGQGTSVTVSSA

SEQ ID NO: 150 - protein, 119 aa, artificial sequence; identical to SEQ ID NO: 149
  primer

SEQ ID NO: 151 - protein, 119 aa, artificial sequence
  primer
EVKLVESGGGSVKPGGSLKLSCAASGFTFSNYLMSWVRQTPEKRLEWVATISSGGSFTYYPDNVKGRFTISRDNAKNTLYLQMSSLKSEDTAMYYCTREFTTKAMDYWGQGTSVTVSSA

SEQ ID NO: 152 - protein, 119 aa, artificial sequence; identical to SEQ ID NO: 134
  primer

SEQ ID NO: 153 - protein, 120 aa, artificial sequence
  primer
  misc_feature (10)..(10): Xaa can be any naturally occurring amino acid
  misc_feature (19)..(19): Xaa can be any naturally occurring amino acid
  misc_feature (40)..(40): Xaa can be any naturally occurring amino acid
  misc_feature (49)..(50): Xaa can be any naturally occurring amino acid
  misc_feature (63)..(63): Xaa can be any naturally occurring amino acid
  misc_feature (68)..(68): Xaa can be any naturally occurring amino acid
  misc_feature (77)..(77): Xaa can be any naturally occurring amino acid
  misc_feature (79)..(79): Xaa can be any naturally occurring amino acid
  misc_feature (101)..(102): Xaa can be any naturally occurring amino acid
  misc_feature (105)..(105): Xaa can be any naturally occurring amino acid
  misc_feature (115)..(115): Xaa can be any naturally occurring amino acid
  misc_feature (120)..(120): Xaa can be any naturally occurring amino acid
EVQLQQSGAXLARPGASVXLSCKASGYTFTDYYIHWVKQXTGQGLEWIXXIYPGTDNTYYNEXFKGKXTLTADKSSXTXYMQLSSLTSEDSAVYFCARQLXXYFXMDYWGQGTSXTVSSX

SEQ ID NO: 154 - protein, 119 aa, artificial sequence
  primer
  misc_feature (103)..(103): Xaa can be any naturally occurring amino acid
EVKLVESGGGSVKPGGSLKLSCAASGFTFSSYLMSWVRQTPEKRLEWVATISSGGSFTYYPDNVKGRFTISRDNAKNTLYLQMSSLKSEDTAMYYCTREFTTXALDYWGQGTSVTVSAA

SEQ ID NO: 155 - protein, 10 aa, artificial sequence
  primer
TREFTTKALD

SEQ ID NO: 156 - protein, 10 aa, artificial sequence
  primer
TREFTTKAMD

SEQ ID NO: 157 - protein, 11 aa, artificial sequence
  primer
ARTATALYTMD

SEQ ID NO: 158 - protein, 7 aa, artificial sequence
  primer
LRTLPCI

SEQ ID NO: 159 - protein, 10 aa, artificial sequence
  primer
SREFTTKAMD

SEQ ID NO: 160 - protein, 11 aa, artificial sequence
  primer
  misc_feature (5)..(6): Xaa can be any naturally occurring amino acid
  misc_feature (9)..(9): Xaa can be any naturally occurring amino acid
ARQLXXYFXMD

SEQ ID NO: 161 - protein, 17 aa, artificial sequence
  primer
TISSGGSFTYYPDNVKG

SEQ ID NO: 162 - protein, 17 aa, artificial sequence
  primer
NIDPYYDSISYNQKFKG

SEQ ID NO: 163 - protein, 17 aa, artificial sequence
  primer
NFDPYYGVITYNQKFKG

SEQ ID NO: 164 - protein, 17 aa, artificial sequence
  primer
TISSGGSYTYYPDNVKG

SEQ ID NO: 165 - protein, 10 aa, artificial sequence
  primer
  misc_feature (1)..(1): Xaa can be any naturally occurring amino acid
  misc_feature (3)..(3): Xaa can be any naturally occurring amino acid
  misc_feature (9)..(9): Xaa can be any naturally occurring amino acid
XFXTDWFYXT

SEQ ID NO: 166 - protein, 17 aa, artificial sequence
  primer
NFDPYYGVISYNQKFKG

SEQ ID NO: 167 - protein, 17 aa, artificial sequence
  primer
TISSGGGFTYYPDNVKG

SEQ ID NO: 168 - protein, 17 aa, artificial sequence
  primer
  misc_feature (1)..(1): Xaa can be any naturally occurring amino acid
  misc_feature (14)..(14): Xaa can be any naturally occurring amino acid
XIYPGTDNTYYNEXFKG

SEQ ID NO: 169 - protein, 112 aa, artificial sequence
  primer
DIVLTQSPASLAVSLGQRATISCRASESVDSYGNSFMHWYQKPGQPPKLLIYLASNLESGVPARFSGSGSRTDFTLTIDPVEADDAATYYCQQNNEDPYTFGGGTKLEIKRA

SEQ ID NO: 170 - protein, 113 aa, artificial sequence
  primer
DIVLTQSPASLAVSLGQRATISCRASESVDSYGNSFMHWYQQKPGQPPKLLIYLASNLEFGVPARFSGSGSRTDFTLTIDPVEADDAATYYCQQNNEDPYTFGGGTKLEIKRA

SEQ ID NO: 171 - protein, 110 aa, artificial sequence; identical to SEQ ID NO: 99
  primer

SEQ ID NO: 172 - protein, 110 aa, artificial sequence
  primer
QIVLTSPAIMSASLGERVTMTCTASSSVSSSYLHWYQQKPGSSPKLWIYSTSNLASGVPARFSGSGSGTSYSLTISSMEAEDAATYYCHQYHRSPPMTFGGGTKLEIKRA

SEQ ID NO: 173 - protein, 113 aa, artificial sequence
  primer
DIVLTQSPASLAVSLGQRATISCRASESVDSYGNSFMHWYQQKPGQPPKLLIYLASNLESGVPARFSGSGSRTDFTLTIDPVEADDAATYYCQQNNEDPYTFGGGTKLEIKRA

SEQ ID NO: 174 - protein, 110 aa, artificial sequence
  primer
ENVLTQSPAIMSASPGEKVTMTCRASSSVSSSYLHWYQQKSGASPKLWIYSTSNLASGVPARFSGSGSGTSYSLTISSVEAEDAATYYCQQYSGYPLTFGSGTKLEIKRA

SEQ ID NO: 175 - protein, 113 aa, artificial sequence
  primer
DNVLTQSPASLAVSLGQRATISCRASESVDSYGNSFMHWYQQKPGQPPKLLIYLASNLESGVPARFSGSGSRTDFTLTIDPVEADDAATYYCQQNNEDPFTFGSGTKLEIKRA

SEQ ID NO: 176 - protein, 112 aa, artificial sequence; identical to SEQ ID NO: 169
  primer

SEQ ID NO: 177 - protein, 113 aa, artificial sequence; identical to SEQ ID NO: 173
  primer

SEQ ID NO: 178 - protein, 111 aa, artificial sequence
  primer
EKLCSPSLQQSCLHLQGQKVTMTCRASSSVGSSYLHWYQQKSGASPKLWIYSTSNLASGVPARFSGSGSGTSYSLTISSVEAEDAATYYCQQYSGYPLTFGGGTKLEIKRA

SEQ ID NO: 179 - protein, 110 aa, artificial sequence
  primer
QIVLTQSPAIMSASLGERVTMTCTASSSVSSSYLHWYQQKPGSSPKLWIYSTSNLASGVPARFSGSGSGTDYSLTISNLEQEDIATYFCQQGNTLPLTFGAGTKLELKRA

SEQ ID NO: 180 - protein, 111 aa, artificial sequence
  primer
  misc_feature (7)..(7): Xaa can be any naturally occurring amino acid
EKLCSPXSPAIMSASPGEKVTMTCRASSSVSSSYLHWYQQKSGASPKLWIYSTSNLASGVPARFSGSGSGTSYSLTISSVEAEDAATYYCQQYSGYPLTFGGGTKLEIKRA

SEQ ID NO: 181 - protein, 113 aa, artificial sequence
  primer
DNVLTQSPASLAVSLGQRATISCRASESVDSYGNSFMHWYQQKPGQPPKLLIYLASNLESGVPARFSGSGSRTDFTLTIDPVEADDAATYYCQQNNEDPYTFGGGTKLEIKRA

SEQ ID NO: 182 - protein, 112 aa, artificial sequence; identical to SEQ ID NO: 169
  primer

SEQ ID NO: 183 - protein, 112 aa, artificial sequence
  primer
DNVLTSPASLAVSLGQRATISCRASESVDSYGNSFMHWYQQKPGQPPKLLIYLASNLESGVPARFSGSGSRTDFTLTIDPVEADDAATYYCQQNNEDPYTFGGGTKLEIKRA

SEQ ID NO: 184 - protein, 112 aa, artificial sequence
  primer
DNVLTQSPASLAVSLGQRATISCRASESVDSYGNSFMHWYQKPGQPPKLLIYLASNLESGVPARFSGSGSRTDFTLTIDPVEADDAATYYCLQNNEDPYTFGGGTKLETKRA

SEQ ID NO: 185 - protein, 112 aa, artificial sequence; identical to SEQ ID NO: 184
  primer

SEQ ID NO: 186 - protein, 113 aa, artificial sequence
  primer
DIVMTQSPASLAVSLGQRATISCRASESVDSYGNSFMHWYQQKPGQPPKLLIYLASNLESGVPARFSGSGSRTDFTLTIDPVEADDAATYYCQQNNEDPFTFGSGTKLEIKRA

SEQ ID NO: 187 - protein, 113 aa, artificial sequence
  primer
DIVLTQSPASLAVSLGQRATISCRASESVDSYGNSFMHWYQQKPGQPPKLLIYLASNLESGVPARFSGSGSRTDFTLTIDPVEADDAATYYCQQNNEDPFTFGSGTKLEIKRA

SEQ ID NO: 188 - protein, 113 aa, artificial sequence
  primer
  misc_feature (79)..(80): Xaa can be any naturally occurring amino acid
DIVLTQSPASLAVSLGQRATISCRASESVDNYGISFMNWFQQKPGQPPKLLIYAASNQGSGVPARFSGSGSGTDFTLNXXPMEEDDTAMYFCQQSKEVPWTFGGGTKLEIKRA

SEQ ID NO: 189 - protein, 94 aa, artificial sequence
  primer
  misc_feature (58)..(58): Xaa can be any naturally occurring amino acid
  misc_feature (68)..(68): Xaa can be any naturally occurring amino acid
  misc_feature (78)..(78): Xaa can be any naturally occurring amino acid
  misc_feature (92)..(92): Xaa can be any naturally occurring amino acid
DIVLTQSPASLAVSLGQRATISCRASESVDSYGNSFMHWYQKPGQPPKLLIYLASNLXSGVPARFSGXGSRTDFTLTXDPVEADDAATYYCXQN

SEQ ID NO: 190 - protein, 9 aa, artificial sequence
  primer
QQNNEDPYT

SEQ ID NO: 191 - protein, 9 aa, artificial sequence
  primer
SGYPLTFGS

SEQ ID NO: 192 - protein, 9 aa, artificial sequence
  primer
HRSPPMTFG

SEQ ID NO: 193 - protein, 9 aa, artificial sequence
  primer
QQNNEDPFT

SEQ ID NO: 194 - protein, 9 aa, artificial sequence
  primer
YSGYPLTFG

SEQ ID NO: 195 - protein, 8 aa, artificial sequence
  primer
NTLPLTFG

SEQ ID NO: 196 - protein, 9 aa, artificial sequence
  primer
QQSKEVPWT

SEQ ID NO: 197 - protein, 10 aa, artificial sequence
  primer
LQNNEDPYTF

SEQ ID NO: 198 - protein, 7 aa, artificial sequence
  primer
LASNLES

SEQ ID NO: 199 - protein, 7 aa, artificial sequence
  primer
LASNLEF

SEQ ID NO: 200 - protein, 7 aa, artificial sequence
  primer
NLASGVP

SEQ ID NO: 201 - protein, 6 aa, artificial sequence
  primer
NLASGV

SEQ ID NO: 202 - protein, 7 aa, artificial sequence
  primer
AASNQGS

SEQ ID NO: 203 - protein, 119 aa, artificial sequence
  primer
EVKLVESGGGSVKPGGSLKLSCAASGFTFSGYLMSWVRQTPEKRLEWVATISSGGSFTYYPDNVKGRFTISRDNAKNTLYLQMSSLKSEDTAMYYCTREFTTKAMDYWGQGTSVTVSSA

SEQ ID NO: 204 - protein, 113 aa, artificial sequence; identical to SEQ ID NO: 173
  primer

SEQ ID NO: 205 - protein, 111 aa, artificial sequence
  primer
EVQLQQSGAELVKPGASVKLSCKASGYTFTSYWMHWVKQRPGHGLEWIGEISPGNGRTNYNEKFKGKATLTVDKSSSTAYMQLSSLTSEDSAVYYCTRLGGLYWGQGTLVT

SEQ ID NO: 206 - protein, 119 aa, artificial sequence; identical to SEQ ID NO: 63
  primer

SEQ ID NO: 207 - protein, 110 aa, artificial sequence
  primer
EIVLTQSPAIMSASLGERVTMTCRASSSVSSSYLHWYQQKPGASPKLWIYSTSNLASGVPARFSGSGSGTSYSLTISSVEAEDAATYYCQQYSGSPLTFGGGTKLEIKRA

(19) United States
(12) Patent Application Publication
(10) Pub. No.: US 2006/0280679 A1
(43) Pub. Date: Dec. 14, 2006

(54) Method of treating autoimmune disease by inducing antigen presentation by tolerance inducing antigen presenting cells

(76) Inventors: Katherine S. Bowdish, Del Mar, CA (US); Anke Kretz-Rommel, San Diego, CA (US); Naveen Dakappagari, San Diego, CA (US)

Correspondence Address: FISH & NEAVE IP GROUP;ROPES & GRAY LLP, ONE INTERNATIONAL PLACE, BOSTON, MA, 02110-2624, US

(21) Appl. No.: 11/016,647

(22) Filed: Dec. 17, 2004

Related U.S. Application Data
(63) Continuation-in-part of application No. PCT/US04/06570, filed on Mar. 4, 2004.
(60) Provisional application No. 60/548,385, filed on Feb. 28, 2004.
(60) Provisional application No. 60/529,500, filed on Dec. 15, 2003.
(60) Provisional application No. 60/451,816, filed on Mar. 4, 2003.

(51) Int. Cl. A61K 51/00; G01N 33/574; A61K 39/395; C07K 16/46; C07K 16/10; C07K 16/12; C07K 16/42

(52) U.S. Cl. 424/1.49; 424/131.1; 530/387.2; 530/388.3; 435/7.23; 424/178.1; 530/391.1

(57) ABSTRACT

Antibodies to antigen presenting cells may be utilized to interfere with the interaction of the antigen presenting cell and immune cells, including T cells. Peptides may be linked to said antibodies thereby generating an immune response to such peptides. Preferably peptides linked to the antibodies are associated with autoimmunity.

### CROSS REFERENCE TO RELATED APPLICATIONS

This application is a continuation-in-part of PCT/US04/06570 filed Mar. 4, 2004, which claims priority to and the benefit of U.S. Provisional Application Ser. No. 60/548,385 filed on Feb. 28, 2004; U.S. Provisional Application Ser. No. 60/529,500 filed Dec. 15, 2003; and U.S. Provisional Application Ser. No. 60/451,816 filed Mar. 4, 2003, the contents of all of which are incorporated herein by reference.

### TECHNICAL FIELD

Developing and restoring natural immune tolerance to autoantigens to treat or prevent autoimmune diseases.

### BACKGROUND OF RELATED ART

T cell-mediated disease insulin-dependent diabetes mellitus (“T1DM”) is a major health problem, affecting more than 1.5 million Americans. This autoimmune disease results from the T cell-mediated destruction of insulin-producing β-cells of the islets of Langerhans within the pancreas. Despite treatment with insulin, deaths resulting from T1DM have increased in the past 20 years, whereas mortality from cancer, cardiovascular disease and stroke have decreased (Hurlbert et al, 2001). In addition, complications of treatment with exogenous insulin including nephropathy, neuropathy and retinopathy are very debilitating.

T1DM is considered a Th1-mediated disease and early intervention which shifts the immune response towards a Th2 type, for example by systemic administration of IL-4, can prevent onset of disease (Cameron et al, 1997). The balance of the effector T cells, Th1 and Th2, may be important in maintaining immune tolerance, and shift in balance can result in autoimmunity. However, protection from autoimmune disease is not an intrinsic property of Th2 cells since Th2 cell lines from NOD mice have also been shown to transfer disease (Pakkala et al, 1997).

The immune system has evolved in complex ways to maintain self-tolerance. The thymus provides an important initial selection of T cells. This selection results in the export, to the periphery, of T-cells which are tolerant to self-antigens present in the thymus. However, many tissue-specific proteins are not expressed at sufficient levels to induce tolerance. For example, islet of Langerhans-reactive T cells have been found in healthy subjects, though presumably of low affinity (Lohman et al. 1996). Several mechanisms of peripheral tolerance complement central tolerance mechanisms in the thymus to keep autoreactive T cells under control. One of the key mediators of peripheral tolerance is the antigen presenting cell (“APC”). APCs such as dendritic cells (“DCs”) and macrophages capture self antigens from other cells and present them to autoreactive T cells to induce T cell tolerance by deletion, anergy and/or generation of regulatory T cells (Heath & Carbone, 2001). The current hypothesis is that immature APCs, such as APCs in the steady-state immune system, tolerize rather that activate T cells presumably due to a lack of co-stimulatory molecules. Hawiger et al. have targeted antigen to the major histocompatibility class II (“MHC II”) pathway of DCs using antibodies to DEC-205, a DC-restricted endocyte receptor (Hawiger et al., 2001). The antigen presentation by these DCs prompted a short burst of CD4+ T cell proliferation, followed by deletion and recipients were rendered tolerant to the antigen, as shown by lack of response to subsequent peptide immunization. In contrast, when antigen targeting was accompanied by a strong DC maturation stimulus such as anti-CD40, immunity was induced.

Dendritic cells can also induce peripheral tolerance by generating regulatory T cells that influence the functions of effector T cells through suppressive cytokines or a contact-dependent mechanism (Roncarolo et al, 2001; Jonuleit et al, 2000; Dhodapkar & Steinman, 2001). A number of different protocols for the induction of regulatory T cells have been developed, generally by means of “suboptimal” T cell stimulation. Suboptimal stimulation of T cells can be accomplished by antigen presentation in the absence of co-stimulation, or inflammation, or by partial blocking of the T cell receptor or its co-receptors CD4 and CD8. The phenotype and mechanism of action of the regulatory T cells is heterogeneous. Many suppressor cells are CD4+CD25+, however it is becoming increasingly clear that in many situations CD4+CD25− cells are equally effective. Other markers identified in the regulatory T cell population include CD62L, GITR and CD103 (Lafaille & Lafaille, 2002), and CD8+ regulatory T cells have also been reported (Dhodapkar & Steinman, 2002). Some regulatory T cells have been shown to produce the immunosuppressive cytokine interleukin (“IL”)-I0 (Wakkach et al, 2001; Barrat et all 2002), while regulatory T cells induced by oral tolerance have been characterized by the production of Transforming Growth Factor-β (“TGF-β”), in addition to the Th2 type cytokines IL-4 and IL-I0 (Weiner, 2001). Contact-dependent suppressor cells have been generated by activating CD4+CD45RA+ human peripheral T cells in the presence of TGF-β (Yamigawa et al, 2001). While induction of regulatory T cells requires stimulation through the T cell receptor, their suppressive effect appears to be non-antigen specific (Thorton & Shevach, 2000).

Immunoregulatory T cells have been shown to play a role in down modulating the pathogenic autoreactive T cells in NOD mice. There is evidence that prediabetic mice harbor immunoregulatory T cells and that a decrease in their numbers, or their functional capacity, is a major contributing event in the disease progression (Sempe et al, 1994). Co-transfer experiments have shown that CD4+ T splenocytes from prediabetic mice fully prevent disease transfer by diabetogenic cells into immuno-incompetent recipients (Boitard et al, 1989; Hutchings & Cooke, 1990). Also, induction of regulatory T cells by immature DCs correlated with disease prevention in the NOD mouse model (Huges et al, 2002).

In humans, autoreactive T cells responding to insulin, glutamic acid decarboxylase (“GAD”), heat shock protein (“HSP”) 60, or protein tyrosine phosphatase-like molecule (“IA-2”), and other undefined β-cell antigens have been described (Roep et al, 1990; Atkinson et al, 1992; Honeyman et al, 1993; Reijonen et al, 2002).

GAD is a biosynthetic enzyme of the inhibitory neurotransmitter gamma animobutyric acid (Baekkeskov et al, 1990). Two distinct isoforms with 65% homology, GAD65 and GAD67, have been cloned. Although GAD65 is the predominant isoform in humans, whereas GAD67 is the major form in NOD mice, antibodies against both isoforms are detected in humans (Kaufman et al, 1992). In NOD mice, anti-GAD antibodies were detected before, or at the time of, insulitis, and before antibodies to other β-cell antigens developed. This timing implies that GAD is the primary antigen that initiates β-cell autoimmunity in this model (Tisch et al, 1993). Further evidence for an important role of GAD in diabetes comes from the observations by many laboratories that GAD-specific T cells isolated from spleen or pancreas of diabetic mice can transfer disease to naive animals (Rohane et al, 1995; Wen et al, 1998; Zekzer et al, 1998). Although there remains controversy with regard to the central role of GAD in the pathogenesis of T1DM, evidence from animal experiments suggests at least an important role of this protein.

Immunization with purified GAD65 at an early age either intrathymically or intravenously can tolerize T cells against pancreatic β-cells in NOD mice, thereby preventing insulitis and diabetes (Tian et al, 1996; Ma et al, 1997). Tolerization against GAD could also prevent the development of immune reactions against other antigens such as HSP65. Further studies addressed which GAD peptides were capable of inducing tolerance (Tisch et al, 2001; Tisch et al, 1999; Zechel et al, 1998). Protection from diabetes onset can also be achieved by either insulin or HSP65 treatment via the intravenous, subcutaneous, oral or nasal route (Elias et al, 1991; Elias & Cohen, 1994; Elias et all 1997; Atkinson et all 1990). While antigen-specific therapies are highly effective in preventing disease onset when administered early, only few attempts were successful at controlling ongoing disease (Elias & Cohen, 1994; Tian et al, 1996).

General peptide immunizations cannot control whether antigen presenting cells present the peptides at a stage that induces immunity or by antigen presenting cells that can shift the immune response towards tolerance, and therefore can result in either immune stimulation or immune suppression.

Compromising the immune system can prevent the development of diabetes. A vast array of general agents suppressing T cell function such as FK506, anti-CD4, anti-CD8, anti-CTLA-4 and others have been shown to prevent or delay diabetes onset in NOD mice (reviewed in: Atkinson & Leiter, 1999). However, none of these reagents is specific for diabetogenic T cells, and the majority of these can prevent onset of disease, but is ineffective once disease is established. General immunosuppressive agents such as cyclosporine tested in clinical trials have been effective short-term (Feutren et al, 1988; Skyler & Rabinovitch, 1992). However, discontinuation of immunosuppression led to prompt relapses, and side effects such as kidney toxicity preclude long-term treatment (Parving et al, 1999).

Clinical trials have been initiated to assess the efficacy of antigen-specific therapy in diabetes. The HSP6O p277 peptide (DiaPep277) was tested in early onset diabetics (Raz et al, 2001). Multiple immunizations with the peptide slowed the disease progression and large-scale studies have been initiated to validate and extend the results. Clinical trials using the beta-chain of human insulin in combination with incomplete Freund's adjuvant, an altered peptide ligand of insulin B9-23 and GAD, are underway. However, trials treating recently diagnosed diabetics with oral insulin failed (Pozzili et al. 2000; Chaillous et al. 2000) and parenteral insulin administration was unsuccessful in preventing disease in high risk prediabetics (Diabetes Prevention Trial-Type 1 (DPT) Study Group, 2002). Failure could be due to several factors including choice of antigen, antigen dose (Kurts et al., 1999), timing and route of administration. Also, antigen therapy can not control what type of immune cell takes up the antigen. While mice are under controlled pathogen-free conditions, this is not the case in human trials. Priming, rather than tolerance can take place when there are concurrent bacterial or viral infections. In animals, diabetes could be induced by antigen immunization under certain conditions (Blana et al. 1996; Bellmann et al. 1998).

Since the understanding of how the immune system maintains tolerance to self-antigens has grown substantially in the past decade, current therapeutic strategies to prevent or cure T1DM aim at restoring immune tolerance to β-cell antigens. Current immunotherapy strategies are aimed at inducing tolerance to β-cell antigens either by directly inactivating the autoreactive T cells and/or inducing T cells with regulatory capabilities. Induction of regulatory T cells appears to be a promising approach for treatment of a number of autoimmune diseases.

### SUMMARY

The present disclosure relates to a method of treating autoimmune disease by inducing immune tolerance. The immune tolerance is induced by presenting autoantigens onto antigen-presenting cells. The autoantigens are linked to antibodies which recognize antigen-internalizing receptors. The autoantigens are internalized by and presented on the antigen-presenting cells, causing an inhibition of autoreactive T cells.

In a particularly useful embodiment, the methods and compounds described herein are used to treat diabetes mellitus by inducing an immune tolerance to an autoantigen, which can be, inter alia, β cell antigens, GAD or an epitope thereof, insulin or an epitope thereof, HSP or an epitope thereof. The autoantigen is linked to an antibody which recognizes DC-SIGNR, or a variation of DC-SIGNR, which is an antigen-internalizing receptor. The autoantigen is internalized into the target liver sinusoidal endothelial cells or other tolerizing APC's expressing DC-SIGNR on the surface. The autoantigen is presented on the target liver sinusoidal endothelial cells and inhibits the proliferation of autoreactive T cells or activates suppressive effects of regulatory T cells.

In another aspect, antibody/peptide constructs are described which contain an antibody to a receptor on an antigen presenting cell linked to a peptide. Preferably the peptide is an antigen, more preferably an autoantigen. In particularly useful embodiments, the antibody/autoantigen construct or portion thereof is internalized by the antigen presenting cell and immune tolerance to the autoantigen is achieved. In some cases a toxin can be combined with the antibodies of the present disclosure and administered to a patient. Where the toxin is to, e.g., a tumor cell, the antibody of the present disclosure can be utilized to direct the toxin to the tumor cell and thereby focus administration of the toxin to the tumor cell.

In another aspect, methods for recombinantly producing engineered antibodies that contain an antibody to a receptor or an antigen presenting cell linked to an autoantigen are described.

The present disclosure also relates to antibodies to DC-SIGNR which interfere with the interaction of DC-SIGNR expressing cells and ICAM-expressing cells such as T cells. Blocking of such interaction might result in immune stimulation. Furthermore, antibodies agonistic for L-SIGN might alter antigen presentation properties of the targeted cell, which could result in either immune activation or suppression.

In another aspect, the antibodies to DC-SIGNR prevent entry of viruses into cells including liver cells such as liver sinusoidal endothelial cells and their infection into other cells. The antibodies to DC-SIGNR may also be utilized to prevent entry of viruses into T-cells and their infection into other cells. In some embodiments, the present disclosure includes the use of antibodies to DC-SIGNR in vaccines.

In other embodiments, antibodies of the present disclosure may be utilized to bind to DC-SIGN and/or L-SIGN, thereby blocking the binding, infection, and transmission of infectious agents including, but not limited to, viruses such as HIV, HCV, Ebola, SARS, CMV and Sindbis.

In yet another embodiment, the antibodies to DC-SIGNR may be utilized to block binding, infection, and transmission of bacteria of the genus Mycobacterium, including M. tuberculosis and M. bovis. In other embodiments, the antibodies to DC-SIGNR may be utilized to block binding, infection, and transmission of parasites such as Schistosoma mansoni.

In yet another embodiment, the antibodies or antibody/peptide constructs of the present disclosure can be labeled with a toxin to DC-SIGNR expressing cells. Administration of the anti-DC-SIGNR antibodies or anti-DC-SIGNR antibody/peptide constructs labeled with toxin can then be utilized to reduce the levels of DC-SIGNR expressing cells which, in some instances, can be beneficial, such as in the treatment of autoimmune disease.

Antibodies to DC-SIGNR of the present disclosure may also be utilized as routine diagnostics for tumor types associated with DC-SIGNR expression and, in some embodiments, may be provided as part of diagnostic kits.

Antibodies to DC-SIGNR of the present disclosure may also be utilized as therapeutics for the treatment of cancer and tumor types associated with DC-SIGNR expression.

Antibodies of the present disclosure may also be utilized to isolate DC-SIGNR expressing cells from cells not expressing DC-SIGNR.

In some embodiments the antibodies to DC-SIGNR of the present disclosure can be a humanized antibody. In other embodiments, the antibodies to DC-SIGNR of the present disclosure can be an scFv.

Further embodiments of the present disclosure relate to prophylactic techniques as well as diagnostic techniques using the compositions and/or embodying the methods as described above. Compositions comprising the antibodies to DC-SIGNR of the present disclosure in a pharmaceutically acceptable carrier are also provided.

### BRIEF DESCRIPTION OF THE DRAWINGS

FIG. 1 schematically shows the interaction an antibody/autoantigen construct in accordance with the present disclosure with an antigen presenting cell (APC), and a T cell.

FIG. 2A shows the light chain amino acid sequences (SEQ. ID NOS: 1-6) and heavy chain amino acid sequences of rabbit anti-mSIGNR1 scFV antibodies.

FIG. 2B shows the heavy chain amino acid sequences (SEQ. ID NOS: 7-12) of rabbit anti-mSIGNR1 scFV antibodies.

FIG. 3 is a schematic diagram of a portion of a vector for antibody peptide construct production.

FIG. 4 is a graphical depiction of the results of in vitro experiments in accordance with the present disclosure showing the reactivity of IgG1 clones with human DC-SIGNR.

FIG. 5 is a graphical depiction of the results of in vitro experiments in accordance with the present disclosure showing the reactivity of IgG2a clones with human DC-SIGNR.

FIG. 6 is a graphical depiction of the results of in vitro experiments in accordance with the present disclosure showing the reactivity of IgG1 clones with human DC-SIGNR and DC-SIGN.

FIG. 7 is a graphical depiction of the results of in vitro experiments in accordance with the present disclosure showing the reactivity of IgG2a clones with human DC-SIGNR and DC-SIGN.

FIGS. 8A-8C shows the amino acid sequences of heavy chain clones reactive with human DC-SIGNR (SEQ. ID NOS: 17-36).

FIGS. 9A-9B shows the amino acid sequences of light chain clones reactive with human DC-SIGNR (SEQ. ID NOS: 37-55).

FIG. 10 shows additional amino acid sequences of IgG1 heavy chain clones reactive with human DC-SIGNR (SEQ. ID NOS: 63-82).

FIG. 11 shows additional amino acid sequences of IgG1 light chain clones reactive with DC-SIGNR (SEQ. ID NOS: 96-115).

FIG. 12 shows additional amino acid sequences of IgG2a heavy chain clones reactive with DC-SIGNR (SEQ. ID NOS: 133-154).

FIG. 13 shows additional amino acid sequences of IgG2a light chain clones reactive with DC-SIGNR (SEQ. ID NOS: 169-189).

FIG. 14 shows that six antibodies (clone names C7, D12, E4, E10, G3, G10) exhibited very good binding with L-SIGN receptor, while three antibodies (D12, G3 and E10) also reacted with DC-SIGN but at substantially lower level.

FIG. 15 shows the affinity of various antibodies for the L-SIGN protein.

FIG. 16 shows the epitope specificity of different antibodies characterized by competing out L-SIGN specific monoclonal antibody (mab162) binding to L-SIGNFc fusion protein in an ELISA.

FIG. 17 shows the results of antibody internalization by liver sinusoidal endothelial cells.

FIG. 18 shows the results of fluorescent beads adhesion assay for ligand blocking used to measure ICAM-1 and ICAM-3-mediated adhesion of K562/LSIGN cells as measured by flow cytometry.

### DETAILED DESCRIPTION

The present methods induce immune tolerance to autoantigens, or self-peptides, implicated in autoimmune disease.

Immunotolerance is induced in accordance with the present disclosure by administering an antibody/autoantigen construct (sometimes referred to herein as an “engineered antibody”) to a subject. The antibody/autoantigen construct includes an autoantigen linked to an antibody.

The antibody component can be an antibody that binds to any receptor on any antigen presenting cell. As those skilled in the art will appreciate, types of antigen presenting cells include dendritic cells, macrophages, endothelial cells Kupffer cells and B cells. Among the presently known receptors or antigen presenting cells are DEC-205, mannose receptor, DC-SIGN, DC-SIGNR, MHC, toll receptor, langerin, asialoglycoprotien receptor, beta-glucan receptor, C-type lectin receptor and dendritic cell immunoreceptor. In particularly useful embodiments, the receptor is one that will internalize the STT antibody. Whether internalization occurs at a particular receptor can be determined experimentally using techniques known to those skilled in the art. Receptors or antigen presenting cells that are presently known to provide internalization of antibodies include DEC-205, mannose receptor, DC-SIGN and DC-SIGNR.

The antibody component can be a natural antibody (isolated using conventional techniques) or an antibody that is synthetically prepared by recombinant methods within the purview of those skilled in the art. The antibody can be, for example, a fully human antibody, a non-human antibody, a humanized antibody, a chimeric antibody or any of the foregoing types of antibodies that have been manipulated in any way (e.g., site-specific modifications or de-immunization). The antibody can be advantageously selected from a library of antibodies using techniques known to those skilled in the art, such as, for example phage display and panning.

Once selected, nucleic acid encoding the antibody can be amplified using techniques known to those skilled in the art such as, for example, conventional PCR or the amplification technique described in U.S. patent application Ser. No. 10/251,085 filed Sep. 19, 2002 and U.S. patent application Ser. No. 10/014,012 filed Dec. 10, 2001, respectively, the disclosures of which are incorporated herein by reference.

An autoantigen is linked to the antibody to prepare an antibody/autoantigen construct in accordance with this disclosure. For purposes of the present disclosure, the terms “antibody/autoantigen construct” and “antibody/peptide” are used interchangeably.

Any autoantigen can be employed. The autoantigen can be naturally occurring and isolated using techniques known to those skilled in the art. Alternatively, if the amino acid sequence of the autoantigen is known, it can be synthetically prepared using known techniques. Suitable autoantigens include insulin, GAD, Hsp, nuclear antigens, acetylcholine receptor, myelin basic protein, myelin oligodendrocyte glycoprotein, proteolipid protein, myelin associated glycoprotein, glomular basement membrane protein and thyrotropin receptor. In particularly useful embodiments, the autoantigen is one that induces immune tolerance upon presentation by a tolerizing antigen presenting cell.

The autoantigen can be linked to the antibody by any suitable method. One particular method is set forth in the Examples, infra, however this disclosure is not limited to any particular method of making the antibody/autoantigen construct.

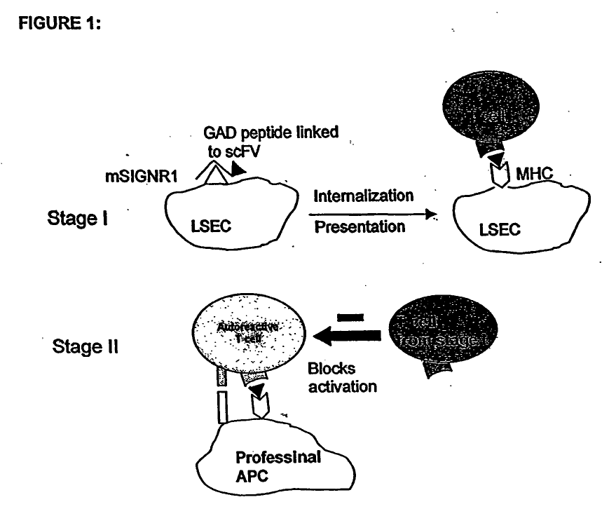

The present methods of inducing immune tolerance to autoantigens target antigen-presenting cells (“APCs”) and direct an autoantigen to those cells by way of an antibody. FIG. 1 schematically shows the interaction of an antibody/autoantigen construct in accordance with the present disclosure with an antigen presenting cell (APC), and a T cell. The antibody recognizes a receptor on the targeted cells. To direct delivery of the autoantigen via the antibody, the two are linked. This linking may be accomplished by any method, although this disclosure delineates the use of vector cloning. The antibody targets and binds to the unique antigen-internalizing receptor only, thereby assuring delivery of the autoantigen to the desired cell type.

After the antibody is bound to the targeted antigen-internalizing receptor, the linked autoantigen and the antibody are internalized in the antigen presenting cell. The autoantigen is presented on the surface of the APCs, presumably through the autoantigen's interaction with major histocompatibility complex (“MHC”) within the cell. Once an autoantigen is expressed on the surface of the APCs with co-stimulatory potential, naive autoreactive T cells can become activated and target and react with their specific autoantigen. The absence of a co-stimulatory molecule in the surface of the APCs is most likely involved in limiting the T cell response. Autoreactive effector T cells can kill only a limited number of antigen expressing tissue cells. After killing a few target cells, the effector cell dies. The autoantigen presenting cells are then tolerated. Presentation of antigen by tolerizing antigen presenting cells to naive T cells induces regulatory T cells. Subsequently, the regulatory T cells prevent activation of other potentially auto-reactive T cells by stimulatory antigen presenting cells.

Thus, in some embodiments, the antibodies of the present disclosure may be utilized to form antibody/autoantigen constructs capable of binding to liver sinusoidal endothelial cells thereby stimulating proliferation of regulatory cells, or the antibodies of the present disclosure may be utilized to form antibody/autoantigen constructs capable of binding to liver sinusoidal endothelial cells thereby suppressing the activity of auto-reactive T-cells. Antibody/autoantigen constructs of the present disclosure may also be used to deliver a vaccine antigen to sinusoidal endothelial cells, including those found in the lymph nodes, thereby stimulating proliferation of antigen specific T-cells.

The present antibody/autoantigen construct can be administered in accordance with known methods, e.g., injection or infusion by intravenous, intraperitoneal, intracerebral, intramuscular, subcutaneous, intraocular, intraarterial, intrathecal, inhalation or intralesional routes, topical or by sustained release systems as noted below. The antibody/autoantigen construct is preferably administered continuously by infusion or by bolus injection. One may administer the antibody/autoantigen construct in a local or systemic manner.

The antibody/autoantigen constructs may be prepared in a mixture with a pharmaceutically acceptable carrier. Techniques for formulation and administration of the compounds of the instant application may be found in “Remington's Pharmaceutical Sciences,” Mack Publishing Co., Easton, Pa., latest edition. This therapeutic composition can be administered intravenously or through the nose or lung, preferably as a liquid or powder aerosol (lyophilized). The composition may also be administered parenterally or subcutaneously as desired. When administered systemically, the therapeutic composition should be sterile, pyrogen-free and in a parenterally acceptable solution having due regard for pH, isotonicity, and stability. These conditions are known to those skilled in the art.

Briefly, dosage formulations of the present antibody/autoantigen construct are prepared for storage or administration by mixing the compound having the desired degree of purity with physiologically acceptable carriers, excipients, or stabilizers. Such materials are non-toxic to the recipients at the dosages and concentrations employed, and may include buffers such as TRIS HCl, phosphate, citrate, acetate and other organic acid salts; antioxidants such as ascorbic acid; low molecular weight (less than about ten residues) peptides such as polyarginine, proteins such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethylene glycol.

When used for in vivo administration, the antibody/autoantigen construct formulation must be sterile and can be formulated according to conventional pharmaceutical practice. This is readily accomplished by filtration through sterile filtration membranes, prior to or following lyophilization and reconstitution. The antibody ordinarily will be stored in lyophilized form or in solution. Other vehicles such as naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

Pharmaceutical compositions suitable for use include compositions wherein one or more antibody/autoantigen constructs are contained in an amount effective to achieve their intended purpose. More specifically, a therapeutically effective amount means an amount of antibody effective to prevent, alleviate or ameliorate symptoms of disease or prolong the survival of the subject being treated. Determination of a therapeutically effective amount is well within the capability of those skilled in the art, especially in light of the detailed disclosure provided herein. Therapeutically effective dosages may be determined by using in vitro and in vivo methods.

An effective amount of antibody/autoantigen construct to be employed therapeutically will depend, for example, upon the therapeutic objectives, the route of administration, and the condition of the patient. In addition, the attending physician takes into consideration various factors known to modify the action of drugs including severity and type of disease, body weight, sex, diet, time and route of administration, other medications and other relevant clinical factors. Accordingly, it will be necessary for the therapist to titer the dosage and modify the route of administration as required to obtain the optimal therapeutic effect. Typically, the clinician will administer antibody/autoantigen construct until a dosage is reached that achieves the desired effect. The progress of this therapy is easily monitored by conventional assays.

For any antibody/autoantigen construct, the therapeutically effective dose can be estimated initially from cell culture assays. For example, a dose can be formulated in animal models to achieve a circulating concentration range that includes the EC₅₀ as determined in cell culture (e.g., the concentration of the test molecule which promotes or inhibits cellular proliferation or differentiation). Such information can be used to more accurately determine useful doses in humans.

Toxicity and therapeutic efficacy of the antibody/autoantigen constructs described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Molecules which exhibit high therapeutic indices are preferred. The data obtained from these cell culture assays and animal studies can be used in formulating a range of dosage for use in human. The dosage of such molecules lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1.).

Dosage amount and interval may be adjusted individually to provide plasma levels of the antibody/autoantigen construct which are sufficient to promote or inhibit cellular proliferation or differentiation or minimal effective concentration (MEC). The MEC will vary for each antibody/autoantigen construct, but can be estimated from in vitro data using described assays. Dosages necessary to achieve the MEC will depend on individual characteristics and route of administration. However, HPLC assays or bioassays can be used to determine plasma concentrations.

Dosage intervals can also be determined using MEC value. Antibody/autoantigen construct molecules should be administered using a regimen which maintains plasma levels above the MEC for 10-90% of the time, preferably between 30-90% and most preferably between 50-90%.

In cases of local administration or selective uptake, the effective local concentration of the antibody/autoantigen construct may not be related to plasma concentration.

A typical daily dosage might range from about 1 μg/kg to up to 1000 mg/kg or more, depending on the factors mentioned above. Typically, the clinician will administer the antibody/autoantigen construct until a dosage is reached that achieves the desired effect. The progress of this therapy is easily monitored by conventional assays.

Depending on the type and severity of the disease, from about 0.001 mg/kg to abut 1000 mg/kg, more preferably about 0.01 mg to 100 mg/kg, more preferably about 0.010 to 20 mg/kg of the antibody/autoantigen construct might be an initial candidate dosage for administration to the patient, whether, for example, by one or more separate administrations, or by continuous infusion. For repeated administrations over several days or longer, depending on the condition, the treatment is repeated until a desired suppression of disease symptoms occurs or the desired improvement in the patient's condition is achieved. However, other dosage regimes may also be useful.

In a particularly useful embodiment, the disclosed methods can be used to treat diabetes mellitus by inducing immune tolerance to insulin-producing β cells of the islets of Langerhans within the pancreas. Autoantigens of these cells are linked to antibodies which recognize the desired antigen-internalizing receptor. Suitable autoantigens for use in this disclosure are β cell antigens, and epitopes, or peptides representing epitopes, of insulin, glutamic acid decarboxylase (“GAD”) and heat shock protein (“HSP”). Linking a set of peptides covering epitopes from insulin, GAD and hsp to an anti-DC-SIGNR antibody has the potential to induce tolerance to all major antigens implicated in T1DM. Other autoantigens may be known and used, or discovered and used, by those skilled in the art.

The antigen-internalizing receptor is presented on specialized APCs. For this method, the antigen-internalizing receptor chosen is DC-SIGNR (dendritic cell-specific intercellular adhesion molecule 3-grabbing nonintergrin related receptor). DC-SIGNR is expressed by liver sinusoidal endothelial cells (“LSEC”), which are liver-resident antigen presenting cells. (¹Pohlmann et al, 2001). DC-SIGNR belongs to the family of pathogen internalization receptors that internalize receptor bound protein and facilitate antigen presentation. (¹Geijtenbeek et al., 2002). It has been shown that presentation of an antigen by LSECs results in an antigen-specific tolerance (Limmer et al., 2000). In contrast to other dendritic cell types that can mature from an immature tolerogenic state to an activating state, liver sinusoidal cells can not be induced to develop into an activating antigen presenting cell (²Knolle et al., 1999). The human DC-SIGNR (also called L-SIGN) homologue to human DC-SIGN shows 77% identity to DC-SIGN at the amino acid level and has the typical domain for internalizing receptors (Bashirova et al, 2001; Soilleux et al, 2000). DC-SIGNR is highly expressed on LSEC and is also found on a sub-population of lymph node macrophage-like cells, but is not expressed by DCs.

For purposes of the present disclosure, the terms “DC-SIGNR” and “L-SIGN” are used interchangeably.

The C-type lectin mouse DC-SIGN (CD209) has recently been identified as a DC-specific receptor. DC-SIGN mediates transendothelial migration of DCs, which enables primary immune responses by initiating transient DC-T cell interactions (³Geijtenbeek et al, 2000; ²Geijtenbeek et al, 2000). DC-SIGN also serves as an internalizing antigen receptor recognizing pathogens through carbohydrate structures. Besides its prominent role in DC-T cell clustering and initiation of T cell responses, DC-SIGN is a major receptor involved in infection of DC and subsequent transmission to T cells of viruses such as HIV-1, HIV-2, SIV-1, hepatitis C virus (HCV), Ebola virus, cytomegalovirus (CMV), and Dengue virus; bacteria such as Helicobacter pylori, Klebsiella pneumonae, and Mycobacteria tuberculosis; yeast such as Candida albicans; and parasites such as Leishmania pifanoi and Schistosoma mansoni. The murine homologue of DC-SIGNR, mSIGNR1, captures antigens that are rapidly internalized and targeted for lysozomes for processing (¹Geijtenbeek et al, 2002). Based on amino acid sequence, murine mSIGNR1 is equally homologous to human DC-SIGNR as it is to human DC-SIGN and is therefore useful for animal modeling studies.

In some embodiments, the antibodies to DC-SIGNR modulate, i.e. inhibit or enhance, the interaction of DC-SIGNR expressing cells with ICAM-expressing cells. In one embodiment, the anti-DC-SIGNR antibodies bind to the DC-SIGNR receptor site on the surface of an antigen presenting cell such as LSEC, and impede the interaction(s) between the LSEC and a T cell. More specifically, the antibodies to DC-SIGNR reduce the adhesion between LSEC and T cells by interfering with the adhesion between DC-SIGNR and an ICAM receptor on the surface of a T cell. Blocking of this interaction can modulate the immune response. For example, the antibodies to DC-SIGNR can modulate the immune function by blocking L-SIGN-T-cell interaction, thereby causing proliferation of regulatory T-cells, including those found in the liver. In another embodiment, the antibodies to DC-SIGNR can modulate the immune function by blocking L-SIGN-T-cell interaction, thereby suppressing proliferation auto-reactive of T-cells, including those found in the lymph nodes.

In some additional embodiments, the antibodies of the present disclosure may be utilized to form antibody-peptide constructs capable of binding to liver sinusoidal endothelial cells thereby stimulating proliferation of regulatory cells, or the antibodies of the present disclosure may be utilized to form antibody-peptide constructs capable of binding to liver sinusoidal endothelial cells thereby suppressing the activity of auto-reactive T-cells. Antibody-peptide constructs of the present disclosure may also be used to deliver a vaccine antigen to sinusoidal endothelial cells, including those found in the lymph nodes, thereby stimulating proliferation of antigen specific T-cells.

As used herein, “ICAM receptor(s)” means both the ICAM-2 and ICAM-3 receptor, especially the ICAM-3 receptor.

In some embodiments, the antibodies to DC-SIGNR of the present disclosure do not bind to DC-SIGN. In other embodiments, the antibodies of the present disclosure possess high affinity for DC-SIGNR and low affinity for DC-SIGN. Antibodies of the present disclosure may be capable of binding both linear and conformational epitopes on DC-SIGNR.

It is also contemplated that the present antibodies or antibody/peptide constructs can be labeled with a toxin to DC-SIGNR expressing cells. Administration of the anti-DC-SIGNR antibodies or anti-DC-SIGNR antibody/peptide constructs labeled with toxin can then be utilized to reduce the levels of DC-SIGNR expressing cells which, in some instances, can be beneficial, such as in the treatment of autoimmune disease, cancer or inflammatory diseases. In this manner, the present antibodies or antibody/peptide constructs can also be utilized to kill or ablate DC-SIGNR expressing cells in vivo. This involves administering the antibodies or antibody/peptide constructs bonded to a cytotoxic drug (e.g., a toxin or radiation-emitting compound) to a subject requiring such treatment. Since the antibodies or antibody/peptide constructs recognize DC-SIGNR expressing cells (e.g., cancer cells or liver sinusoidal endothelial cells), any such cells to which the antibodies or antibody/peptide constructs bind are destroyed. In one embodiment, a method of treating cancer in accordance with this disclosure involves administering an effective cancer-cell killing amount of an anti-DC-SIGNR antibody or anti-DC-SIGNR antibody/peptide construct having a toxin bound thereto to a cancer patient. In another embodiment, a method of treating an inflammatory disease in accordance with this disclosure involves administering an effective DC-SIGNR expressing cell killing amount of an anti-DC-SIGNR antibody or anti-DC-SIGNR antibody/peptide construct having a toxin bound thereto to a patient suffering from an inflammatory disease.

In other embodiments, the antibodies to DC-SIGNR of the present disclosure block entry of viruses into liver cells such as liver sinusoidal cells and their infection into other cells. The antibodies to DC-SIGNR of the present disclosure may also block entry of viruses into T-cells and their infection into other cells.

In some embodiments, the antibodies to DC-SIGNR of the present disclosure may be utilized to block infection by bacteria of the genus Mycobacterium. Infections which may be blocked include those caused by M. tuberculosis and M. bovis.

By interfering with the adhesion of T cells to antigen presenting cells, the use of antibodies to DC-SIGNR will affect antigen presenting cell-T cell clustering, T cell activation and other interactions that rely on contact between antigen presenting cells and T cells. These other interactions include both direct cell-to-cell contact or close proximity of antigen presenting cells and T cells.

In other embodiments, the anti-DC-SIGNR antibodies of the present disclosure are linked to peptides such as autoantigens, or self-antigen, peptides. These peptides can be linked to anti-DC-SIGNR antibodies by any suitable method, including grafting a vector to an antibody fragment and cloning the linked vector/antibody, or chemically linking. Methods of linking a vector, cloning or chemical linking are well known to those skilled in the art.

The peptides, preferably autoantigens, along with the linked antibody, are then internalized into the LSEC. LSECs bear surface molecules necessary for antigen presentation such as MHC II, CD80 and CD86 (Lohse et al, 1996; Rubinstein et al, 1986). In addition to inducing a regulatory phenotype in naive CD4+T cells (Knolle et al, 1999), LSECs can induce tolerance in CD8+ T cells by cross-presenting exogenous antigen (Limmer et al, 2000). LSECs respond to stimuli as TNF-α and endotoxin by downregulation of MHC, and hindering endosomal processing (²Knolle et al, 1999). Furthermore, LSECs do not migrate out of the liver to lymph organs.

This internalization facilitates the presentation of self-antigen peptides to the surface to the LSECs, mediated via MHC interactions. Once an autoantigen is expressed on the surface of the LSECs which have co-stimulatory potential, naïve autoreactive T cells can become activated. The T cells target and react with the linked autoantigen. The effector T cells kill few LSECs and die off without co-stimulatory molecules. This presentation of an autoantigen by LSEC results in autoantigen-specific tolerance.

The liver has a unique microenvironment with an abundance of tolerogenic mediators such as IL-10 and TGF-β and specialized APCs that favor the development of immunologic tolerance (¹Knolle & Gerken, 2000). Tolerogenic properties of the liver are supported by the finding that allogeneic liver transplants can be accepted across MHC barriers (Calne, 1969). Furthermore, application of antigens via the portal vein is more likely to lead to tolerance than systemic application of the antigen (Kamei et al, 1990). Draining through the liver has been reported to be a prerequisite for oral tolerance induction (Yang et al, 1994). Blood passing through the hepatic vessels first comes into contact with Kupffer cells and LSECs. The blood flow through the hepatic sinusoids is slow, allowing contact between the liver sinusoidal cell populations and passing leukocytes. LSECs bear surface molecules necessary for antigen presentation such as MHCII, CD80 and CD86 (Lohse et al, 1996; Rubinstein et al, 1986). In addition to inducing a regulatory phenotype in naive CD4+ T cells (³Knolle et al, 1999), LSECs can induce tolerance in CD8+ T cells by cross-presenting exogenous antigen (Limmer et al, 2000). Klugewitz et al (Klugewitz et al, 2002) demonstrated that injection of Th1, IFN-γ producing TCR-transgenic cells into mice results after intravenous protein immunization in suppression of IFN-γ production by these cells in the liver and promotion of Th2-cells. In contrast to professional myeloid APC that can differentiate from an immature, tolerogenic stage into a mature stage initiating immunity, LSECs respond to stimuli as TNF-α and endotoxin by downregulation of MHC and hindering endosomal processing (²Knolle et al, 1999). Furthermore, LSECs do not migrate out of the liver to lymph organs. LSECs might not be the only APC specialized on inducing tolerance. Pugliese et al recently identified a small subset of spleen DCs that induced tolerance by presenting endogenously expressed autoantigen (Puglise et al, 2001). Overall, LSECs appear to be a favorable cell type for presenting β-cell antigens with the purpose of tolerance induction.

In addition, both DC-SIGN and L-SIGN have been shown to bind to a number of viruses, e.g., HIV (Bashirova et al. 2001; ²Pohlmann et al. 2001), HCV (Gardner et al. 2003), Ebola (Alvarez et al. 2002; Simmons et al. 2003), SARS (Jeffers et al. 2004), CMV (Halary et al. 2002), and Sindbis (Klimstra et al. 2003). While these receptors act as an attachment point for HIV and HCV, transmitting them in trans to other cells, e.g., T-cells (Bashirova et al. 2001; ²Pohlmann et al. 2001; Cormier et al. 2004), they also bind to and permit the entry and infection by Ebola, SARS, CMV and Sindbis viruses into receptor expressing cells. In addition, both DC-SIGN and L-SIGN serve as receptors for infection by bacterial pathogens of the Mycobacterium genus, including M. tuberculosis (⁴Geijtenbeek et al. 2003; Koppel et al. 2004), as well as parasites such as Schistosoma mansoni (Van Liempt et al. 2004).

Thus, in another embodiment, antibodies of the present disclosure may be utilized to bind to DC-SIGN and/or L-SIGN, thereby blocking the binding, infection, and transmission of infectious agents including, but not limited to, viruses such as HIV, HCV, Ebola, SARS, CMV and Sindbis; bacterial pathogens of the Mycobacterium genus, including M. tuberculosis and M. bovis; and parasites such as Schistosoma mansoni.

Antibodies to DC-SIGNR of the present disclosure may also be utilized as routine diagnostics for tumor types associated with DC-SIGNR expression. For example, upregulation of DC-SIGNR in cancer samples could be utilized as the basis for a diagnostic tool to evaluate whether a cancer has become exposed to the immune system, since upregulation of immune receptors is expected to happen only under pressure by the immune system. Using methods known to those skilled in the art, including immunohistochemistry and/or FACS analysis, tumor biopsies may be exposed to antibodies to DC-SIGNR and then analyzed for the presence of bound antibody, which would be indicative of a cancer associated with DC-SIGNR expression. In some embodiments the antibodies may be provided as part of diagnostic kits for determining the presence of a cancer expressing DC-SIGNR.

Antibodies to DC-SIGNR of the present disclosure may also be utilized as therapeutics for the treatment of cancer. In one embodiment, the antibodies of the present disclosure induce ADCC (antibody-dependent cellular cytotoxicity) or CDC (complement-dependent cytotoxicity) of tumor cells, thereby killing said cells.

The present antibodies or antibody/peptide constructs can also be utilized to kill or ablate cancerous cells in vivo. This involves administering the antibodies or antibody/peptide constructs bonded to a cytotoxic drug to a subject requiring such treatment. Since the antibodies or antibody/peptide constructs recognize cancer cells, any such cells to which the antibodies or antibody/peptide constructs bind are destroyed.

The antibodies or antibody/peptide constructs of the present disclosure may be used to deliver a variety of cytotoxic compounds. Any cytotoxic compound can be fused to the present antibodies or antibody/peptide constructs. The fusion can be achieved chemically or genetically (e.g., via expression as a single, fused molecule). The cytotoxic compound can be a biological, such as a polypeptide, or a small molecule. As those skilled in the art will appreciate, for small molecules, chemical fusion is used, while for biological compounds, either chemical or genetic fusion can be employed.

The antibodies or antibody/peptide constructs of the present disclosure may be used to deliver a variety of cytotoxic drugs including therapeutic drugs; a compound emitting radiation; molecules of plant, fungal, or bacterial origin; biological proteins; and mixtures thereof. The cytotoxic drugs can be intracellularly acting cytotoxic drugs, such as short-range radiation emitters, including, for example, short-range, high-energy α-emitters. Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, α-sacrin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin, for example. Procedures for preparing enzymatically active polypeptides of the immunotoxins are described in WO84/03508 and WO85/03508, which are hereby incorporated by reference. Certain cytotoxic moieties are derived from adriamycin, chlorambucil, daunomycin, methotrexate, neocarzinostatin, and platinum, for example.

Procedures for conjugating the antibodies or antibody/peptide constructs with the cytotoxic agents have been previously described.

Alternatively, the antibodies or antibody/peptide constructs of the present disclosure can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp. 303-316 (Academic Press 1985), which is hereby incorporated by reference. Other suitable radioisotopes include α-emitters, such as ²¹²Bi, ²¹³Bi, and ²¹¹At, and β-emitters, such as ¹⁸⁶Re and ⁹⁰Y. Radiotherapy is expected to be particularly effective in connection with prostate cancer, because prostate cancer is a relatively radiosensitive tumor.

In another embodiment, the antibodies to L-SIGN of the present disclosure may be utilized as therapeutics to treat tumors by binding to L-SIGN and preventing negative regulation of the immune system through L-SIGN expressing cancer cells. By preventing this negative regulation, the immune system may proceed to eradicate the cancer cells.

The efficacy of the above treatments may be confirmed utilizing methods known to those skilled in the art, including xenograft models.

Antibodies to L-SIGN in accordance with the present disclosure which are utilized as cancer therapeutics may also be combined with any other immunomodulatory therapy, such as cancer vaccines, anti-CTLA-4, anti-CD25 or cyclophosphamide to achieve increased therapeutic efficacy in the treatment of cancer.

Antibodies to L-SIGN in accordance with the present disclosure may also be used in some embodiments to isolate L-SIGN expressing cells found in a mixture of cells which includes cells not expressing L-SIGN. For example, human non-parenchymal liver cells, which contain a mixture of sinusoidal endothelial cells, red blood cells, Kupffer cells and other minor cell populations may be readily obtained from commercial sources. To isolate L-SIGN expressing cells, i.e., liver sinusoidal cells, from human non-parenchymal liver cells, the red blood cells may be exposed to an agent such as ammonium chloride which will result in lysis of the red blood cells. The dead cells may be removed using commercially available dead cell removal kits, including those sold by Miltenyi Biotech (Germany). The remaining cells may be counted and labeled with anti-L-SIGN antibody. After washing the cells, they may be resuspended in an appropriate buffer and the cells of interest, i.e., the L-SIGN expressing cells, may be isolated by exposing the remaining cells to anti-L-SIGN antibody which, in some cases, may be conjugated to a bead or similar separation medium. Commercially available separation media and methods for their use are known to those skilled in the art and include commercially available beads from Miltenyi. In some particularly useful embodiments, the L-SIGN expressing cells may be isolated from cells not expressing L-SIGN by using anti-LSIGN-conjugated beads, e.g., anti-mouse IgG-conjugated beads, according to the manufacturer's instructions (Miltenyi). Those cells binding to the anti-L-SIGN antibody are L-SIGN expressing cells and are thereby isolated from the remaining cells not expressing L-SIGN. Once isolated from the remaining cells, the L-SIGN expressing cells may be removed from the anti-L-SIGN antibody using methods readily known to those skilled in the art. The quality of the isolation may be monitored by methods known to those skilled in the art, including FACS analysis using a panel of antibodies against molecules such as CD54, mannose receptor, LYVE-1, CD40, asialoglycoprotein and others.

Practice of the present methods, including additional preferred aspects and embodiments thereof, will be more fully understood from the following examples, which are presented for illustration only and should not be construed as limiting in any way.

### EXAMPLE 1

### Obtaining Anti-mSIGNR1 Antibodies

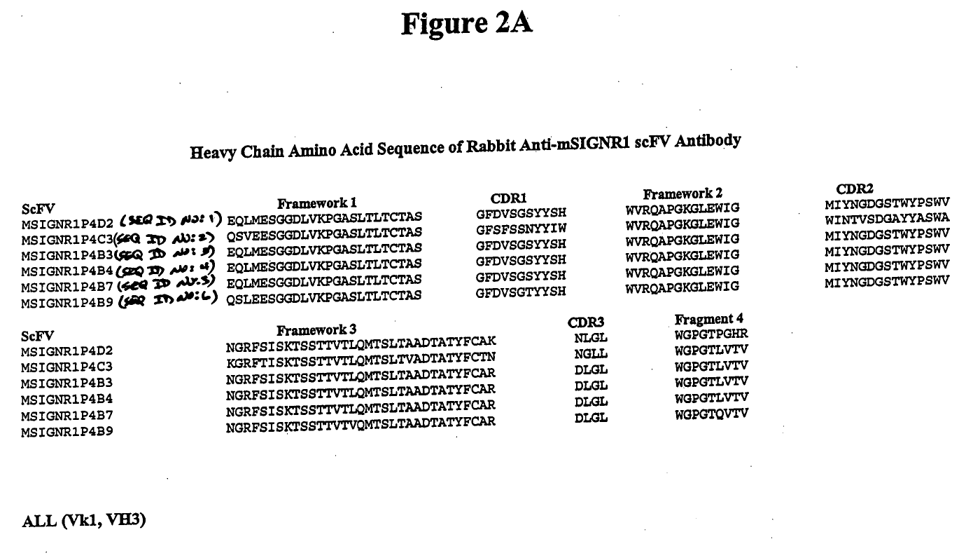

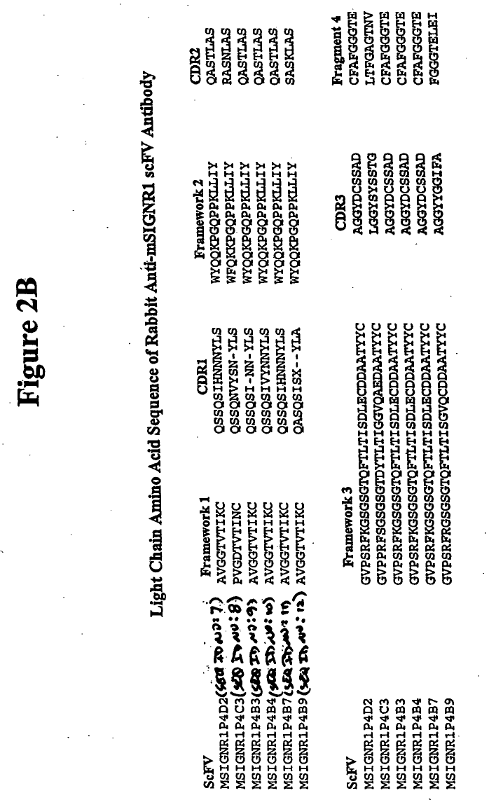

Using phage display technology, a panel of single chain antibodies (scFv) that recognize mSIGNR1 was identified. scFvs contain the variable light and heavy chain region connected by a linker. Their short length makes these antibody fragments very suitable for antigen linkage, and the capacity for binding to the receptor is preserved. Rabbits were immunized with recombinant mSIGNR1, and a scFv antibody library was constructed using the phage display vector pRL4 which is described in Published International Application No. WO 02/46436 A2 published on Jun. 13, 2002, the disclosure of which is incorporated herein by reference. Antibody fragments in this system are displayed on the gene III coat protein of the phage. Antibodies recognizing mSIGNR1 were isolated by 4 rounds of solid phase panning on recombinant mSIGNR1. Six different antibodies were identified. The amino acid sequences of these six antibodies are presented in FIGS. 2A and B (SEQ. ID NOS: 1-6 and 7-12, respectively). All antibodies recognized mSIGNR1 in solid phase ELISA, and no cross-reactivity with mDC-SIGN, the murine homologue of human DC-SIGN, was observed. The antibodies were epitope-tagged with HA and HIS₆. Both mSIGNR1 and DC-SIGNHIS were produced by 3T3 EBNA cells and purified over a nickel column.

### EXAMPLE 2

### Identifying Anti-mSIGNR1 Antibodies that are Internalized upon Binding to the Cell Surface Receptor

Screen for Cell Lines Expressing mSIGNR1

A panel of murine macrophage cell lines (P388D1, 1-13.35, WEHI-3 and J774) are screened for expression of mSIGNR1 by RT-PCR by standard methods. Primers are designed based on the mSIGNR1 Genbank sequence and used these in RT-PCR of mouse organs. A cell line expressing mSIGNR1 on the mRNA level is identified and surface expression is confirmed by FACS analysis. 5×10⁵ cells are incubated with 1 μg anti-mSIGNR1 antibody in PBS containing 1% BSA and 0.1% NaN₃ on ice for 15 minutes, conditions that do not allow for antibody internalization. After 2 washes with PBS containing 1% BSA and 0.1% NaN₃, bound anti-mSIGNR1 are detected by biotinylated anti-HA (Roche) followed by PE-conjugated streptavidin (Becton Dickenson) and cells are analyzed using FACSCalibur (Becton Dickinson, Mountain View, Calif.). Alternatively, internalization is determined on primary cells known to express mSIGNR1 such as liver sinusoidal endothelial cells. Expression of mSIGNR1 on LSECs can also be confirmed by FACS as described above, but only 1×10⁵ cells is added per reaction.

Measurement of Internalization

Once a mSIGNR1-expressing cell line or primary cell type has been identified, internalization of the antibody panel is assessed by FACS analysis. To show that internalization is based on mSIGNR1 binding, a cell line that does not express mSIGNR1 such as JAWS1 mouse dendritic cells is included. Anti-mSIGNR1 detection using biotinylated anti-HA antibody followed by PE-conjugated steptavidin on intact and permeabilized cells is compared as described for anti-DEC-205 antibodies (Mahnke et al, 2000). 1.5×10⁶ cells for cell lines or 3×10⁵ cells for primary cells are incubated with 3 μg of mSIGNR1 in PBS containing 1% bovine serum albumin (BSA) for 20 minutes at 4° C. to allow for antibody binding to the surface without internalization. Unbound antibody is removed by washing 2 times with PBS containing 1% BSA at 4° C. Each sample is divided into 3. One third is fixed with 4% paraformaldehyde and surface antibody is detected as described above. The other two thirds are further incubated for 30 minutes at 37° C. to allow for internalization before being fixed. One half is directly detected with anti-HA and steptavidin, the other half is permeabilized by incubating the cells with PBS containing 0.1% (vol/wt) saponin (Sigma-Aldrich). The amount of internalized antibody is calculated by subtracting the mean fluorescence in fixed cells from that recorded with fixed and permeabilized cells. The antibodies with the highest percentages of internalization within 30 minutes are chosen for further studies linking peptides to the antibodies. An existing unrelated rabbit scFv is used as a negative control, the commercially available ER-TR9 antibody that has recently been shown to bind mSIGNR1 (¹Geijtenbeek et al, 2002) is used as a positive control. Also, Fab fragments of ER-TR9 are produced by papain digestion and tested for internalization to verify that dimerization is not a requirement for internalization. If desired, the scFvs can be converted into Fab′2 or IgG.

In an alternative embodiment, a mSIGNR1 library is panned for internalizing antibody as described by the group of James D. Marks (Poul et al., 2000). A suitable process for this embodiment is outlined below.

Selection of Internalizing Antibodies from mSIGNR1 Phage Library

5×10⁶ cells identified as described above to express mSIGNR1 are incubated with 1×10¹² colony forming units of phage from a mSIGNR1 library presenting antibody fragments fused to gene 3 protein on their surface for 1.5 hours at 4° C. to allow phage binding without internalization. After phage binding, the cells are washed 5 times with phosphate-buffered saline to remove non-specifically or weakly bound phage. Cells are then incubated for 15 minutes at 37° C. to allow endocytosis of surface-bound phage, but avoid phage degradation within the cell. To remove phage bound to the surface of the cell, cells are stripped by washing three times with a low pH glycine buffer. Then cells are trypsinized and washed with PBS before being lysed with high pH triethylamine. The cell lysate containing phage are used to infect E. coli to prepare phage for the next round of selection. A total of three rounds of selection are performed. The titer of phage bound to the cell surface (found in the first low pH glycine wash) and the number of phage recovered from within the cell are monitored for each round. An increase in the number of endocytosed phage indicates a successful selection of internalizing phage antibody.

To determine whether any of the internalized scFv antibody fragments bind to mSIGNR1, 500 clones from round 3 are selected using a robotic Qpix (Genetix) system and grown in 96-well dishes in SB medium overnight in a HiGrow shaker (Gene Machines). The next day, dishes are spun down and supernatants tested in solid phase mSIGNR1 ELISA using a robotic Genesis freedom 200 (Tecan) system. 96-well ELISA plates are coated with 1 μg mSIGNR1/ml PBS overnight at 4° C. The next day, plates are blocked by 1% BSA, followed by 3 washes with PBS containing 0.05% Tween. Control plates are coated with 1% BSA. Supernatants containing antibody are added to mSIGNR1 or BSA alone wells at concentrations between 0.05-5 μg/ml in PBS containing 1% BSA. After 2 hours on a shaker at room temperature, plates are washed 3 times with PBS containing 0.05% Tween. For detection of bound scFv, anti-HA antibody (12CA5 mouse ascites, Strategic Biosolutions, DE) are added at a 1:1,000 dilution in PBS with 1% BSA. After 2 hours on a shaker at room temperature, plates are washed again and alkaline-phosphatase-conjugated anti-mouse IgG (Sigma) are added for 2 hours. After 3 more washes, bound antibody are detected using Sigma 104® (substrate. The plates are read at various time points at OD₄₀₅ with an ELSA plate reader (Molecular Devices).

Clones giving a positive signal in ELISA are characterized by restriction enzyme digest pattern. DNA are isolated using Qiagen's miniprep kit. 2 μg of DNA are digested with 5 U of EcoRII for 2 hours at 37° C. and then the samples are run on a 4% NuSieve agarose gel. Patterns are compared and sequences are purified in small quantities (about 100-300 μg). scFvs are properly assembled in the periplasmic space of bacteria and are secreted. scFvs can either be isolated from the supernatant or the periplasmic space. Clones are grown in 4 liter of SB to an OD₆₀₀ of 0.8 and induced with 1 mM isopropyl-p-D-thiogalactopyranoside (IPTG) for 3-4 hours at 30° C. to produce optimum amounts of scFv. To isolate single chain antibodies form the periplasmic space, cell pellets are resuspended in cold PBS with added Complete Mini (Roche) protease inhibitor and are sonicated using a Sonics Vibra-cell VC750. Cellular debris is then pelleted and the supernatants are applied to Qiagen Ni-NTA columns using an Akta FPLC (Pharmacia). Antibody is eluted with imidazole. This method generally yields about 100-300 μg of purified antibody/liter. Endotoxin is removed by filtration through Sartorius Q15 filters generally yielding antibody preparations containing less than 10 U/ml endotoxin as determined by LAL test (an assay commercially available from Bio Whittaker). Antibodies are analyzed again for internalization as described above as well as for binding to recombinant mSIGNR1 in solid phase ELISA. The antibody with the highest percentage of internalization within 30 minutes and a good signal in a solid phase ELISA (>10 D after 1 hour at 1 μg/ml) are selected to make peptide-antibody constructs.

### EXAMPLE 3

### Link GAD Peptides to the Antibody

Vector and Cloning Strategy

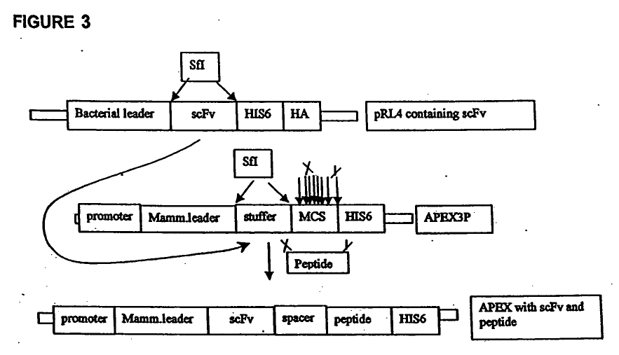

Following identification of the best bacterially-produced scFv, a conversion to a mammalian expression system is made. Mammalian expression allows for the appropriate secondary modifications of the peptides and endotoxin-free production. A vector (e.g., described in U.S. Pat. No. 6,355,245, the disclosure of which is incorporated herein by reference) with compatible restriction sites as shown in FIG. 3 is used. DNA from the antibody of interest in pRL4 (described above) are cut with Sfi and inserted into the Apex 3P vector containing a CMV promoter and mammalian antibody leader sequence. To insert nucleotides encoding the peptides of interest, restriction sites are chosen from the sites available (MCS=Nael, FseI, XbaI, EcoRI, PstI, EcoRV, BSABI , BstXI, NotI, BsrBI, Xho, PbvIOI, SphI, NsiI, XbaI) that are not contained within the antibody sequence. Oligonucleotides encoding peptides are synthesized by Operon with the appropriate restriction sites at each end and are inserted using T4 DNA ligase. The resulting construct will contain the scFv followed by a spacer determined by the restriction enzyme chosen followed by a peptide and a HIS-tag (FIG. 3). Sequences are confirmed using standard techniques before transfecting DNA into 393EBNA cells for antibody production.

Choice of Peptide

While it should be understood that any of the peptides of the known diabetes autoantigens insulin, hsp and GAD 65 and 67 can be used in the processes described herein, for the following experiment GAD is chosen as the peptide. GAD-reactive T cells are the first autoreactive T cells to be detected in the NOD mouse (Tisch et all 1993; Kaufman et al, 1993) and have been shown to be important in the disease process. Furthermore, human and murine GAD are 95% homologous. Epitopes recognized by splenic NOD T cells have been extensively characterized (Kaufman et al, 1993; Tisch et all 1999; Zechel et al. 1998) and many immunodominant peptides are similar in NOD mice and T1DM patients and have been used interchangeably in in vitro T cell assays (Kaufman et all 1993). The initial immune response in NOD mice is directed against a defined region in the carboxy-terminal region of GAD65 (peptide 509-528, peptide 524-543 (Kaufman et all 1993). Later T cell responses are also directed against other regions between 200-300 as well as other autoantigens. One of the early CD4 GAD65 T cell epitopes, peptide 524-543 (SRLSKVAPVIKARMMEYGGT (SEQ. ID NO: 13), same sequence in mice and humans) and two of the later occurring murine GAD65 epitopes, peptide 247-266 (NMYAMLIARYKMFPEVKEKG (SEQ. ID NO: 14), 1 amino acid difference between human and mouse underlined), and peptide 290-309 (ALGIGTDSVILIKCDERGK (SEQ. ID NO: 15), same sequence in mice and humans) are selected for use as the peptides in this experiment. All 3 epitopes can induce a spontaneous proliferative response in NOD splenocytes. Furthermore, peptide immunization with peptide 247-266 and peptide 290-309 have been shown to delay diabetes onset in NOD mice (Ma et all 1997; Tisch et all 1999; Zechel et all 1998). In addition to CD4 T cell epitopes, tolerance to CD8 T cell epitopes has also been reported to be important (Quinn et all 2001; Bercovici et al, 2000). As a negative control, an antibody construct is made with hen egg lysozyme peptide 116-124 (KGTDVQAWI) (SEQ. ID NO: 16). The most effective peptides from these in vitro studies are then linked in various combinations with an antibody construct and tested in the NOD diabetes model.

### EXAMPLE 4

### Antibody-Peptide Construct Production and Purification

For T cell experiments, approximately 300 μg of each antibody construct is produced in EBNA293 human embryonic kidney cells. Cells are grown in DMEM with 10% FCS, 2 mM glutamine and 250 U/ml G418 (Sigma). Cells in TI75 flasks are transfected with DNA using Qiagen's Effectine reagent according to the manufacturer's instruction. Medium is exchanged for serum-free medium after 3 days. Supernatant is collected at day 4 and day 8, cell debris is removed by centrifugation and the cleared supernatant is loaded on a Ni-column using a Akta-FPLC. Antibody is eluted with imidazole, dialyzed into PBS and correct size verified by running 1 μg on a SDS-gel.

### EXAMPLE 5

### Internalization of the Antibody-Peptide Construct by LSEC Resulting in Peptide Presentation and the Effect of this Presentation on T Cells

Liver sinusoidal cells are targeted in vitro with the peptide-antibody construct and it is determined whether these cells can induce a phenotypic change in T cells derived from young NOD or Balb Ic mice.

Isolation of Murine Sinusoidal Endothelial Cells

Liver sinusoidal endothelial cells are isolated from 3 week old NOD or 4-6 week-old Balb/c mice. Cells are obtained by portal perfusion first with EGTA to chelate calcium and loosen cell-cell contacts followed by perfusion with 0.05% collagenase A in Hank's buffer to degrade intercellular matrix as described by Kretz-Rommel (Kretz-Rommel & Boelsterli, 1995). The perfused liver is removed from the mouse and gently worked with a pair of angled forceps. The resulting crude cell suspension is filtered through a series of metal sieves (30, 50, 80 mesh) to remove larger tissue fragments. Sinusoidal cells are separated from parenchymal cells by density gradient centrifugation on a metrizamide gradient (1.089 g/cm3) followed by 2 washing steps to remove cell debris (3Knolle et al, 1999). At this point, a mixture of Kupffer cells and liver sinusoidal cells is obtained. For FACS experiments this is sufficient, since Kupffer cells can be distinguished from liver sinusoidal endothelial cells using the F4/80 antibody that recognizes Kupffer cells, but not liver sinusoidal cells. However, for co-culture experiments with T cells and peptide, Kupffer cells are removed by labeling the cells with PE-conjugated F4/80 (BD Pharmingen) followed by Miltenyi's anti-PE microbeads and magnetic sorting of the labeled cells using MACS column and separator according to the manufacturer's instructions. The remaining cell population is seeded onto 96 well tissue culture plates in Dulbecco's modified Eagle medium (DMEM) supplemented with 10% fetal bovine serum and 2% glutamine. The purity of cell populations is investigated at day 3 after isolation by FACS staining for surface markers using anti-mSIGNR1 and anti-F4/80. mSIGNR1 is absent on Kupffer cells (¹Geijtenbeek et al, 2002). 90% purity is considered sufficient to proceed with the experiments. 2 mouse livers are used per experiment with an expected yield of about 2×10⁷ cells (Knook & Sleyster, 1976, extrapolated for mouse).

T Cell Phenotypic Assays

T cell assays demonstrate if the peptide-antibody construct results in presentation of peptide by liver sinusoidal endothelial cells and whether peptide presentation can induce a phenotypic change in T cells. Liver sinusoidal endothelial cells are cultured in flat bottom microtiter plates at a density of 1×10⁵ cells/well. After maintaining the sinusoidal cells for 3 days, CD4+ T cells will be purified as described above from a 3-week old and an 8-week old NOD mouse or a 4-6 week-old Balb/c mouse and are added at 10⁴ or 10⁵ cells/well. Also, each antibody-peptide construct at concentrations of 0.1-5 pg/well is added. As a positive control, each GAD and control peptide by itself are included. Peptides are synthesized by SynPep (Dublin, Calif.). Negative control wells include either T cells alone or liver sinusoidal cells alone.

There are 4 possible outcomes of peptide presentation by LSECs to T cells: 1) induction of regulatory T cells characterized by the production of TGF-P and/or IL-I0 and IL-4 or the expression of CD4+CD25+CD62L, 2) deletion of T cells or 3) a complete lack or response. 4) It is also conceivable that peptide presentation instead of inducing tolerance results in stimulation of Th1 cells producing IL-2. To distinguish among these possibilities, culture supernatants (100 pl each) are collected at 24 and 48 hours and assayed for cytokine production as described below. T cell responses of 3-week old are compared with those of 8-week old NOD mice as well as with T cells derived from Balb/c mice that do not show a spontaneous response to GAD. Assays are set up in triplicate and repeated twice. Since a mixture of T cells is used, a cytokine response in the supernatant might not be easily seen. However, a mixture of cells reflects the situation in vivo and GAD-specific T cell responses are seen using total splenocytes (Tisch et al, 1993).

As a more sensitive measure for the induction of regulatory T cells, cells are evaluated for the expression of typical surface markers such as CD25, CD4 and CD62L (Lafaille & Lafaille, 2002) by FACS analysis after 3 days in culture. All reagents are available from BD-Pharmingen. Also, IL-4 production is analyzed by FACS. A functional test of potential immunoregulatory properties of the LSEC/peptide exposed T cells as described below are the ultimate test for tolerance induction in this system. The possibility of peptide presented by LSEC inducing cell death are assessed in culture supernatants using Roche's cell death ELISA kit according to the manufacturer's instructions.

Isolation of Splenocytes and CD4+ T Cells

Spleens from NOD of Balb/c mice are removed in a sterile environment and put in PBS using techniques within the purview of those skilled in the art. Cells are separated using 18-21 gauge needles, and larger pieces are allowed to settle. Supernatant is removed and centrifuged at 200 g for 7 min. Red blood cells are lysed using 5 ml 0.83% NH₄Cl per spleen. Cells are washed twice in PBS and then resuspended in medium. For certain experiments, total splenocytes are used. For other experiments, CD4+ T cells are isolated using Miltenyi's (Auburn, Calif.) CD4+ T cell isolation kit according to the manufacturer's instruction. Magnetic isolation of various cell populations is within the purview of one skilled in the art. In one process, isolation is based on depletion of non-CD4+ T cells using a cocktail of biotin-conjugated monoclonal antibodies against CD8a, CDI 1b, CD45R, DX5 and Ter-1 19. The purity of the isolated population is assessed by staining of a mixture of FITC-conjugated anti-CD4, PE-conjugated anti-CD8, APC-conjugated anti-CD11 b and cy-5-conjugated CD45R (all eBioscience, San Diego, Calif.). Expected purity is 90-95% with 70% yield. Since at least 1×10⁸ cells can be obtained from a mouse spleen and about 25% of splenocytes are CD4+, about 1.75×10⁷ cells can be obtained, enough for 175 96-well microtiter plate wells.

Measurement of Cytokine Production

Presence of IL-10, TGF-P, IFN-γ, IL-4 and IL-2 in supernatants of T cell/LSEC co-cultures are determined by standard sandwich ELISA as described (Kretz-Rommel & Rubin, 1997). All antibody pairs are available from BD Pharmingen. A cytokine capture antibody is coated on the plate in PBS overnight at 4° C. After 3 washes with PBS/0.05% Tween, culture supernatants and a standard curve of mouse recombinant cytokine are added and incubated for 2 hours on a shaker at room temperature. Plates are washed again and bound cytokine is detected with an alkaline-phosphatase conjugated anti-cytokine antibody. After 3 washes, Sigma ₁₀₄™ substrate is added and the plates are read at various timepoints at OD₄₀₅ with an ELISA plate reader (Molecular Devices).

### EXAMPLE 6

### Assess Whether T Cells Exposed to GAD Peptides Presented on LSECs Can Subsequently Prevent Activation of Autoreactive T Cells by Professional APCs Presenting GAD

In a further experiment, whether T cells exposed to peptides on liver sinusoidal endothelial cells for 3 days can negatively regulate activation of autoreactive T cells by peptide presented on splenic professional APCs is tested. Feasibility of the induction of T cells with regulatory properties in vitro has been demonstrated by a number of laboratories (Wakkach et all 2001; Barrat et all 2002; Thorton & Shevach, 1998). Immunosuppressive properties can be tested by adding the regulatory T cells to a culture system in which immune stimulation is normally observed. Addition of GAD peptide to spleen cells from a 7 week-old NOD mouse comprising both APCs and T cells provides such an immunostimulatory system as seen by a strong proliferative response. Addition of regulatory T cells abrogates this response. 10⁵ or 10⁶ splenocytes are added together with either 0.1, 1, or 10 μm peptide per well containing T cells exposed to LSEC and peptide-coupled antibody. Control wells include splenocytes with peptide alone and LSEC+T cells alone. Furthermore, to exclude significant contribution of proliferation by the presumed regulatory T cells, control wells also contain irradiated splenocytes (600 RAD, performed at UCSD irradiation service facility by Joe Aguilera) and T cells previously exposed to LSEC and the peptide-antibody construct. Irradiated splenocytes can present antigen, but do not proliferate. 1 μCi ³H-thymidine is added to each well during the last 16 hours of a 72 hour culture period to label newly synthesized DNA as a readout for proliferation. Cells are harvested using Packard's Universal Cell Harvester and incorporated ³H-thymidine is assessed using a Topcount (Packard). If ³H-thymidine incorporation in cultures containing splenocytes and T cells previously exposed to LSEC+peptide is reduced compared to the splenocyte cultures, T cells have been successfully induced with regulatory properties. Whether the peptide-conjugated anti-mSIGNR1 antibody can induce regulatory T cells in the NOD mouse, and whether disease can be halted are also tested.

### EXAMPLE 7

Mouse anti human L-SIGN antibodies were identified using recombinant phage technology. Mouse libraries (IgG1k and IgG2ak) derived from heavy and light chain combination of mice immunized with recombinant human L-SIGN were prepared by the methods disclosed in WO 03/025202, the contents of which are incorporated by reference herein. Once prepared, the libraries were first panned on human DC-SIGN to remove antibodies cross reactive with DC-SIGN. The unbound supernatants were used for selecting clones uniquely reactive with L-SIGN. A total of ninety-five colonies (36/round) for each of the two libraries (IgG1 & IgG2a) were induced and antibody production and their reactivity with L-SIGN were determined by a capture ELISA. Briefly, anti-human Fc (Caltag) was coated on ELISA plates at 500 ng/ml overnight. The plates were blocked with PBS containing 1% BSA followed by the addition of recombinant L-SIGN at 2 μg/ml. After washing the plate with PBS, supernatants were added. After a 12 hour incubation at room temperature, plates were washed 3 times and an alkaline-phosphatase-conjugated anti-Fab antibody was added for 2 hours. Signal after addition of SigmaS substrate was assessed using an ELISA reader (Molecular Devices).

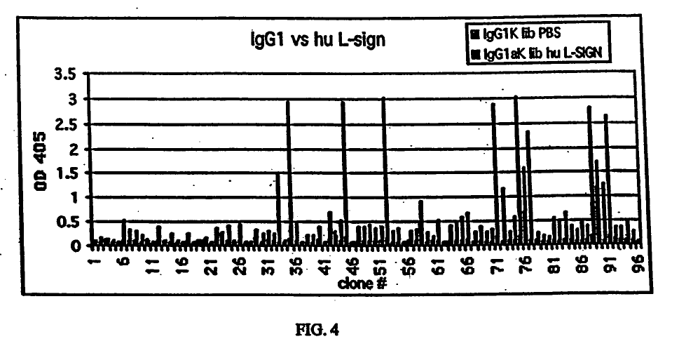

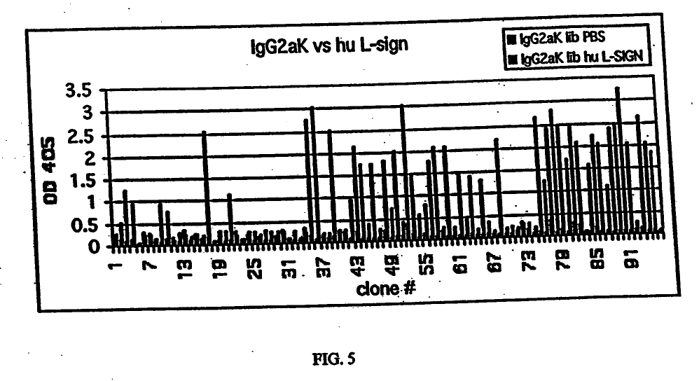

The majority of the clones showed good binding (OD405>1.0) of the antibody on the phage. Several clones from both IgG1 and IgG2a libraries showed positive reactivity with human L-SIGN. Results are set forth in FIGS. 4 and 5: FIG. 4 sets forth the IgG1 clones with human L-SIGN; FIG. 5 sets forth the reactivity of IgG2a clones with human L-SIGN.

### EXAMPLE 8

To identify clones uniquely reactive with human L-SIGN, all clones from Example 7 with OD values of five-fold above background were selected to test their reactivity with human DC-SIGN by ELISA. Anti-human Fc (Caltag) was coated on ELISA plates at 500 ng/ml overnight. The plates were blocked with PBS containing 1% BSA followed by the addition of recombinant DC-SIGN at 2 μg/ml. After washing the plate with PBS, supernatants were added. After a 12 hour incubation at room temperature, plates were washed 3 times and an alkaline-phosphatase-conjugated anti-Fab antibody was added for 2 hours. Signal after addition of SigmaS substrate was assessed using an ELISA reader (Molecular Devices).

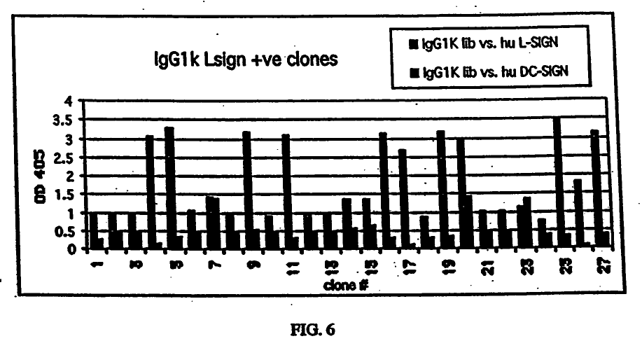

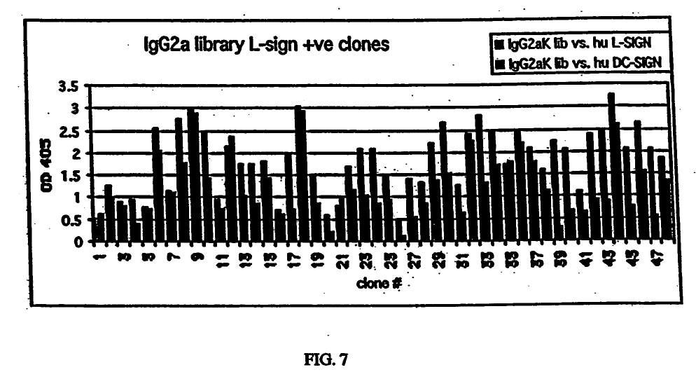

Ten clones from the IgG1 library and three clones from the IgG2a library were found to uniquely react with human L-SIGN (five to ten fold higher OD values vs. DC-SIGN). Results are set forth in FIGS. 6 and 7: FIG. 6 sets forth the reactivity of the IgG1 positive clones with L-SIGN and DC-SIGN; FIG. 7 sets forth the reactivity of the IgG2a clones with L-SIGN and DC-SIGN.

### EXAMPLE 9

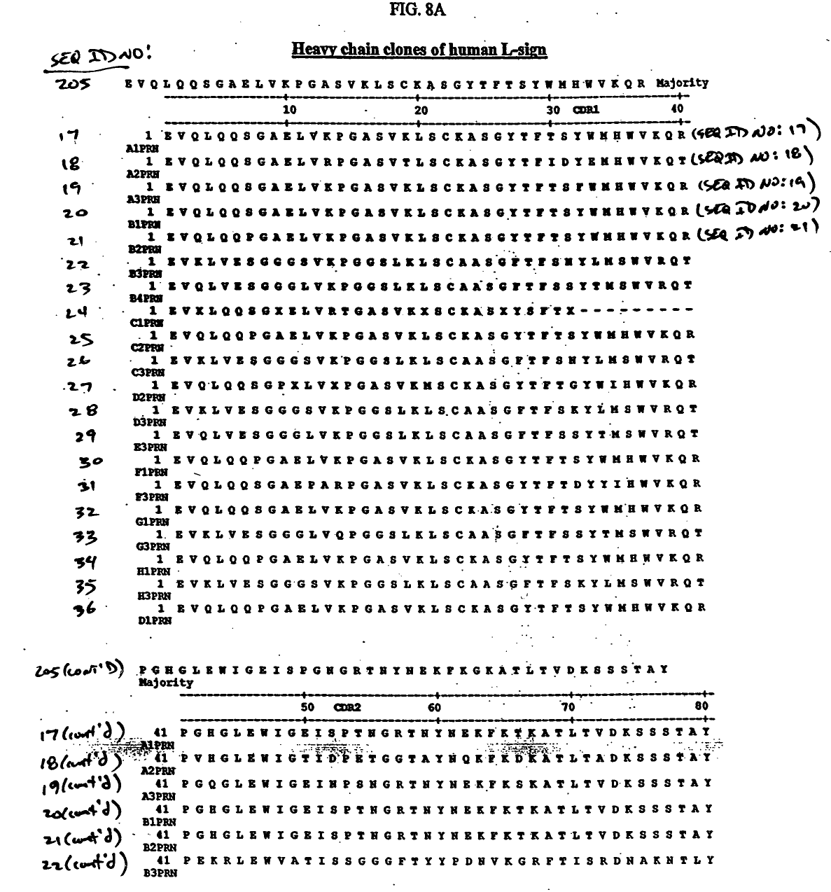

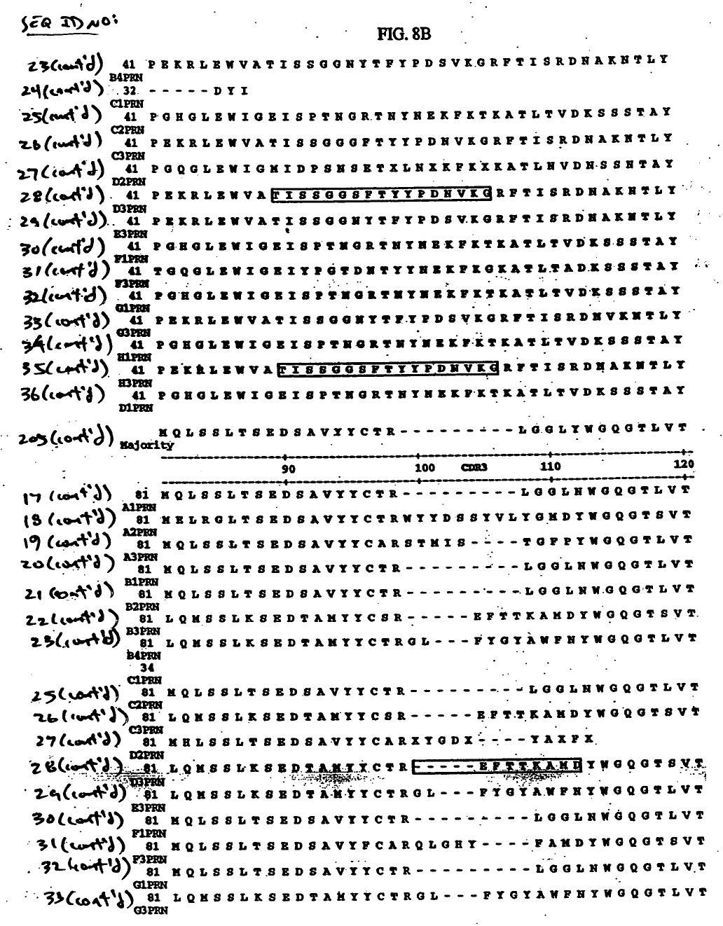

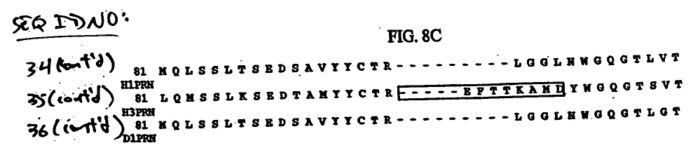

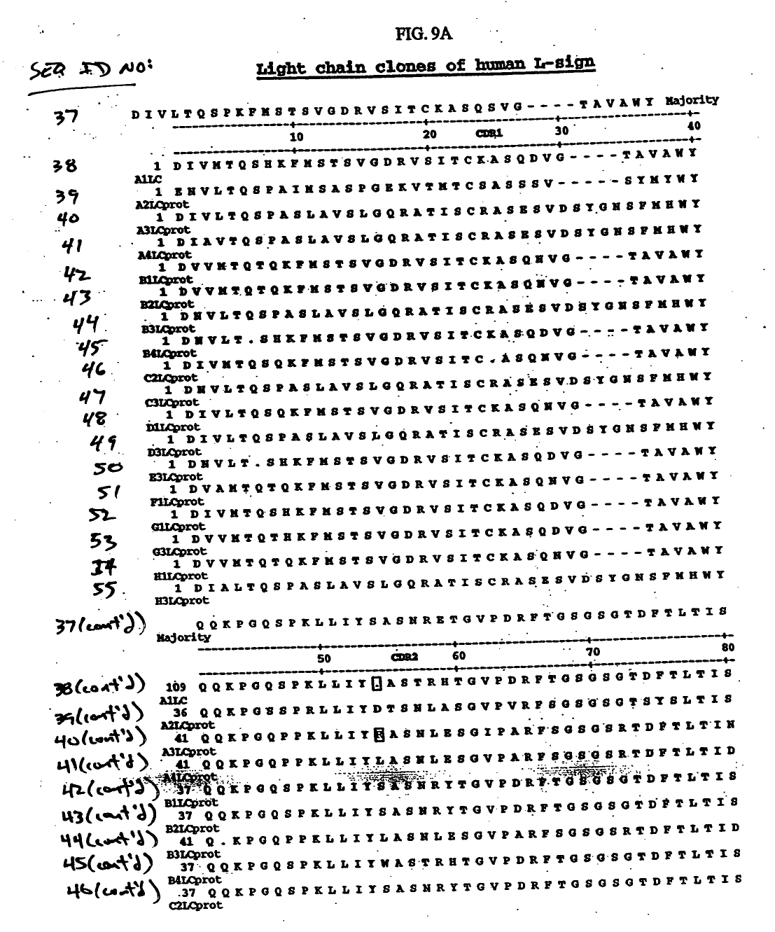

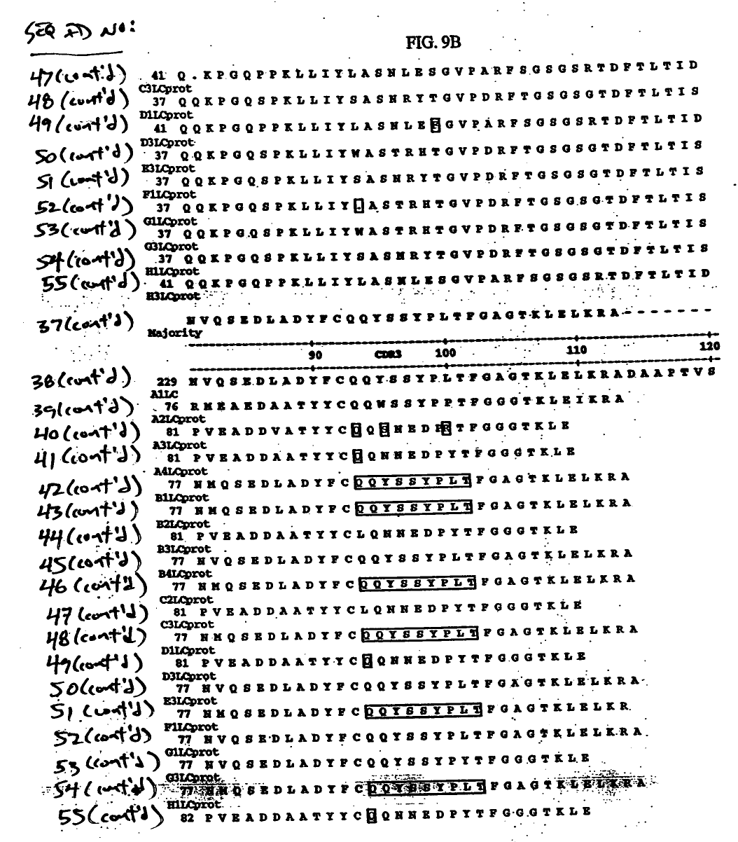

Thirteen clones identified as reactive with only human L-SIGN and nine clones strongly reactive with both L-SIGN and DC-SIGN as identified above in Example 8 were sequenced to determine the number of unique clones. Sequencing was determined by techniques known to those skilled in the art. The sequences of these clones are set forth in FIGS. 8 and 9; sequences for heavy chain clones are set forth in FIG. 8A-8C (SEQ. ID NOS. 17-36); sequences for light chain clones are set forth in FIGS. 9A-9B (SEQ. ID NOS: 37-55).

Sequencing results demonstrated a more diverse group of heavy chains compared with light chains. The diversity of the antibody clones was also enhanced by cross pairing of different light chains with the same heavy chain. A total of five unique clones reactive with L-SIGN were identified and clones were grouped based upon the similarity of their amino acid, sequences (see Table 1 below).

TABLE 1
Grouping of antibody clones reactive with human L-SIGN based
on amino acid sequence similarities

| Ab | | | Unique L-sign |
|---|---|---|---|
| clusters | Light chain | Heavy chain | clones |
| 1 | B1; B2; D1; F1; H1 | [symbol] ^{a} [symbol] ^{a}; B1(identical) | 2 |
| | C2 (stop/CDR1) | B2; C2; D1; F1; H1 | |
| | | (1/FR1 vs. A1, B1, G1) | |
| 2.1^{c} | B3; C3 | B3; C3^{a} | 1 |
| 2.2 | D3(1/CDR2); H3 | D3; H3^{b} | 0 |
| 2.3 | A3 (1/CDR2); A4^{b} | A3^{a} | 1 |
| | (1/CDR3) | | |
| 3.1 | B4; E3 (stop/FR1) | B4^{b}, E3^{b} | 0 |
| | G3 (1/CDR3) | G3^{b} (2/FR1; 1/FR2) | |
| 3.2 | [symbol] | | |
| | (stop/CDR2) | | |
| 4 | | D2^{a} | 1 |
| 5 | A2 | A2^{b} | 0 |
| 6 | | F3^{b} | 0 |
| 7 | | C1^{b} (incomplete seq) | 0 |

^{a}Antibody clones reactive with only L-SIGN
^{b}Antibody clones reactive with both L- and DC-SIGN
^{c}2.1 . . . 2.3 designates similar light chains but unique heavy chains

The reactivities (OD values) of the clones selected for sequencing with L-SIGN and DC-SIGN are set forth below in Table 2.

TABLE 2

| IgG1k | | | IgG1k | IgG2ak | | | IgG2ak |
|---|---|---|---|---|---|---|---|
| library | | | library | library | | | library |
| Seq. | | | clones to | Seq. | | | clones to |
| Well # | deep well# | OD405 | purify | Well # | deep well# | OD405 | purify |
| A1 | A5 | 3 | A5 | A3 | E11 | 2.2 | E11 |
| B1 | C5 | 3.2 | | B3 | F11 | 2 | |
| C1 | B6* | 1.4 | | C3 | F12 | 2 | F12 |
| D1 | E6 | 3.2 | | D3 | A3* | 2 | |
| E1 | D7 | 3.1 | | E3 | D5* | 2.9 | |
| F1 | G9 | 3.1 | | F3 | C6* | 2.3 | |
| G1 | H9 | 2.6 | | G3 | D7* | 2.9 | D7 |
| H1 | C10 | 3.1 | | H3 | E10* | 2.2 | |
| A2 | C11* | 1.4 | | A4 | A11* | 2.2 | |
| B2 | H11 | 3.5 | H11 | B4 | B12* | 2.6 | |
| C2 | B12 | 1.8 | | | | | |
| D2 | C12 | 3.1 | C12 | | | | |

*= Those clones selected for sequencing with L-SIGN and DC-SIGN

As set forth in FIG. 8, heavy chain CDR3 regions of the antibodies that bind to human DC-SIGNR were found have one of the following amino acid sequences: LGGL (SEQ. ID NO: 56); EFTTKAMD (SEQ. ID NO: 57); GLFYGYAWFN (SEQ. ID NO: 58). As set forth in FIG. 9, light chain CDR3 regions of the antibodies that bind to human DC-SIGNR were found to have one of the following amino acid sequences: QQYSSYPLT (SEQ. ID NO:59); QQSNEDPRT (SEQ. ID NO: 60); QQNNEDPYT (SEQ. ID NO: 61); LQNNEDPYT (SEQ. ID NO: 62).

### EXAMPLE 10

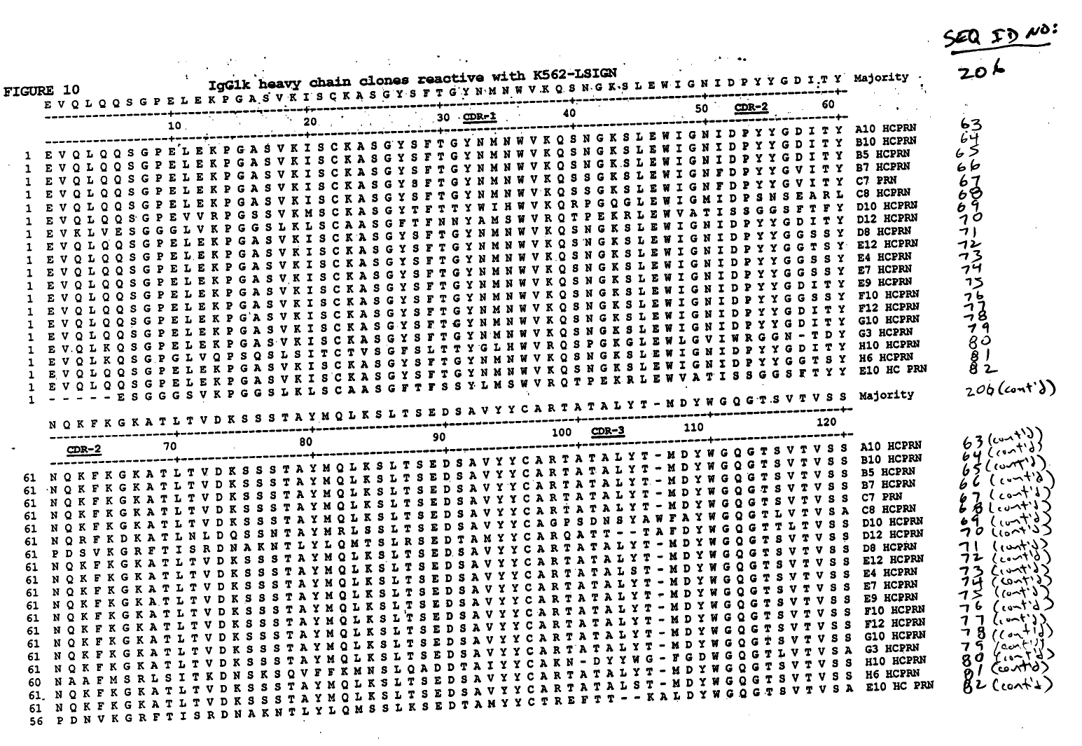

Additional clones from Example 7 above were examined for their ability to bind to L-SIGN utilizing the procedures described above in Example 7. Sequencing was determined by techniques known to those skilled in the art. The sequences for these additional clones are set forth in FIG. 10 (SEQ. ID NOS: 63-82). As set forth in FIG. 10, five additional heavy chain CDR3 regions of the antibodies that bind to human DC-SIGNR were found have one of the following amino acid sequences: PSDNSYAWFA (SEQ. ID NO: 83); QATTTAFD (SEQ. ID NO: 84); TATALSTMD (SEQ. ID NO: 85); NDYYWGFG (SEQ. ID NO: 86); TATALYTMD (SEQ. ID NO: 87); and EFTTKALD (SEQ. ID NO: 88). The CDR2 regions of these clones that bound to human DC-SIGNR were found to have one of the following amino acid sequences: MIDPSNSEARLNQRFKD (SEQ. ID NO: 89); TISSGGSFTFYPDSVKG (SEQ. ID NO: 90); NIDPYYGGTSYNQKFKG (SEQ. ID NO: 91); VIWRGGNTDYNAAFMS (SEQ. ID NO: 92); NFDPYYGVITYNQKFKG (SEQ. ID NO: 93); NIDPYYGGSSYNQKFKG (SEQ. ID NO: 94); and TISSGGSFTYYPDNVKG (SEQ. ID NO: 95).

Table 3 shows additional IgG1k antibody clones selected based on their reactivity with cells expressing L-SIGN.

TABLE 3

| Ab | Light | | |
|---|---|---|---|
| clusters | chain | Heavy chain | Unique clones |
| 1 | A10, H10 | A10, B10, B5, D12, E9, F12, G10, | A10, H10 |
| | | H10 | |
| 2 | D8, F10 | D8, E4, E7, F10 | D8, F10 |
| 3 | E12, H6 | E12, H6 | E12, H6 |
| 4 | B7, C7 | B7, C7 | B7, C7 |
| 5 | C8 | C8 | C8 (stop/LC) |
| 6 | D10 | D10 | D10 |
| 7 | E10 | E10 | E10 |
| 8 | G3 | G3 | G3 (stop/LC) |
| 9 | B5 | — | B5 (stop/LC) |
| 10 | B10 | — | B10 |
| 11 | D12 | — | D12 |
| 12 | E4 | — | E4 |
| 13 | E7 | — | E7 |
| 14 | E9 | — | E9 |
| 15 | F12 | — | F12 |
| 16 | G10 | — | G10 |

Table 4 below shows the reactivity of additional IgG1k antibody clones with cells expressing LSIGN (Geometric Mean fluorescence) and recombinant L-SIGN and DC-SIGN proteins (OD values).

TABLE 4

| | Geo. Mean | Geo. | | |
|---|---|---|---|---|
| IgG1k | Fluorescence | Mean Fluorescence | OD405 | OD405 |
| Clone Name | K562 | K562/L-SIGN | L-SIGN | DC-SIGN |
| A10 | 3.4 | 21.3 | 2.9 | 0.1 |
| B5 | 1.5 | 10.5 | 2.1 | 0.1 |
| B7 | 2.2 | 90.9 | 2.7 | 0.1 |
| B10 | 2.0 | 53.1 | 3.5 | 0.1 |
| C7 | 2.0 | 101.0 | 3.5 | 0.1 |
| C8 | 1.7 | 13.4 | 0.1 | 0.1 |
| D8 | 1.8 | 17.3 | 2.1 | 0.1 |
| D10 | 1.6 | 10.2 | 0.9 | 0.5 |
| D12 | 2.0 | 152.0 | 3.5 | 0.1 |
| E4 | 1.9 | 50.4 | 3.5 | 0.1 |
| E7 | 1.8 | 19.3 | 1.4 | 0.1 |
| E9 | 1.9 | 25.5 | 2.9 | 0.1 |
| E10 | 1.7 | 27.2 | 2.6 | 0.6 |
| E12 | 3.0 | 22.9 | 2.9 | 0.1 |
| F10 | 2.4 | 13.8 | 0.8 | 0.1 |
| F12 | 2.8 | 168.7 | 3.5 | 0.1 |
| G1 | 2.1 | 14.1 | 2.0 | 0.1 |
| G3 | 1.6 | 41.5 | 0.4 | 0.1 |
| G10 | 2.0 | 86.1 | 2.0 | 0.1 |
| H6 | 2.4 | 26.2 | 3.5 | 0.1 |
| H10 | 3.4 | 12.3 | 2.7 | 0.1 |

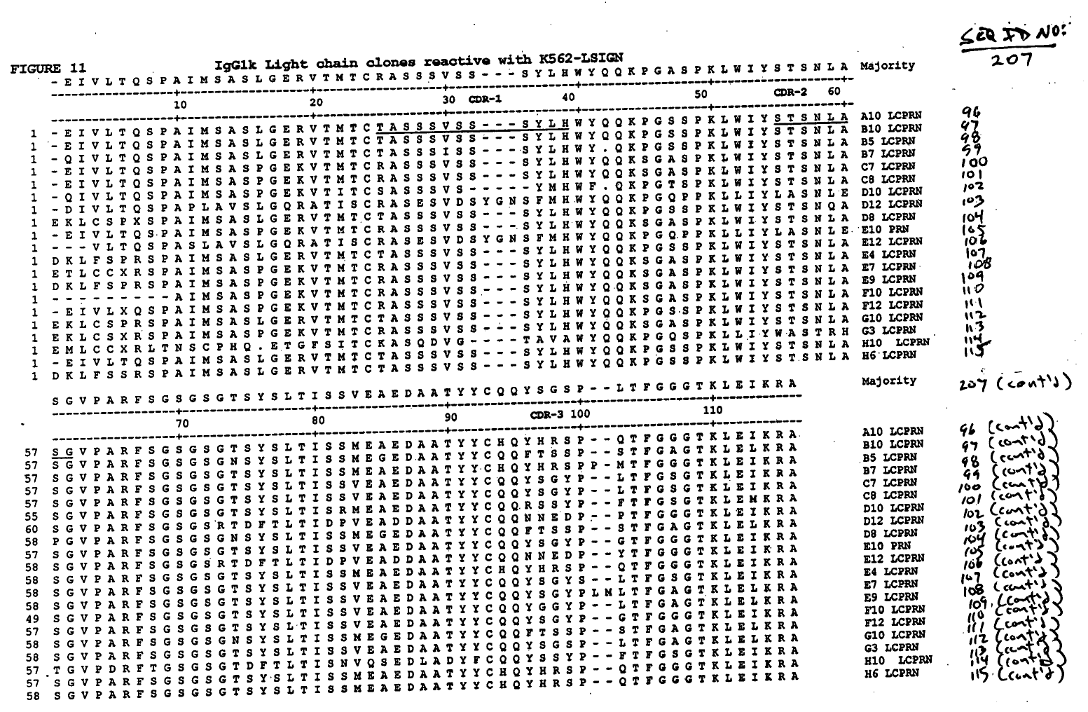

As set forth in FIG. 11, additional clones that bind human DC-SIGNR were identified (SEQ. ID NOS: 96-115). These clones were found to have IgG1k light chain CDR3 regions with one of the following amino acid sequences: Q Y H R S P Q T SEQ. ID NO: 116); C Q Q F T S S P S (SEQ. ID NO: 117); Q Q Y S G Y P L T (SEQ. ID NO: 118); Q Q Y S G Y P G T (SEQ. ID NO: 119); H Q Y H R S P P M T (SEQ. ID NO: 120); Q Q R S S Y P F T (SEQ. ID NO: 121); Q Q Y S S Y P F T (SEQ. ID NO: 122); Q Q N N E D P P T (SEQ. ID NO: 123); Q Q Y S G Y S L T (SEQ. ID NO: 124); Q Q Y S G Y P L M L T (SEQ. ID NO: 125); Q Q Y G G Y P L T (SEQ. ID NO: 126); Q Q N N E D P Y T (SEQ. ID NO: 127); Q Q Y S G S P L T (SEQ. ID NO: 128). The CDR2 regions of these clones that bound to human DC-SIGNR were found to have one of the following amino acid sequences: S T S N L A S G (SEQ. ID NO: 129); L A S N L E S G (SEQ. ID NO: 130); S N Q A P G (SEQ. ID NO: 131); W A S T R H T G (SEQ. ID NO: 132).

Table 5 shows additional IgG2ak antibody clones selected based on their reactivity with cells expressing L-SIGN.

TABLE 5

| Ab | | | |
|---|---|---|---|
| clusters | Light chain | Heavy chain | Unique clones |
| 1 | A12*, B11, | A12, C10, C12, H7 | A12, C12, H7 |
| | C12*, C6, E12, E8*, | | |
| | F10*, H7* | | |
| 2 | F6*, F12* | F12, F6 | F12, F6 |
| 3 | C10, G10, G5 | A3, F10, G5 | A3 |
| 4 | A4, B9 | A4, C7, D12 | A4 |
| 5 | A3 | A5, D8 | C7 |
| 6 | A5 | C5* | D12 |
| 7 | C7 | C6 | F10 |
| 8 | D8 | B9 | G5 |
| 9 | D12 | E12 | C5* |
| 10 | H6 | H6 | H6 |
| 11 | — | G10 | B9 |
| 12 | — | B11 | E12 |
| 13 | — | E8 | G10 |
| 14 | | | B11 |
| 15 | | | E8 |
| 16 | | | C6 |
| 17 | | | A5 |
| 18 | | | D8 |
| 19 | | | C10 |

*These sequences contain a stop codon

Table 6 shows the reactivity of additional IgG2ak antibody clones with cells expressing LSIGN (Geometric Mean fluorescence) and recombinant L-SIGN and DC-SIGN proteins (OD values).

TABLE 6

| | Geo. Mean | Geo. | | |
|---|---|---|---|---|
| IgG2ak | Fluorescence | Mean Fluorescence | OD405 | OD405 |
| Clone Name | K562 | K562/L-SIGN | L-SIGN | DC-SIGN |
| A3 | 2.8 | 15.4 | 3.5 | 1.0 |
| A4 | 2.8 | 12.1 | 3.5 | 0.1 |
| A5 | 1.8 | 59.8 | 3.5 | 0.1 |
| A12 | 2.3 | 23.4 | 3.5 | 0.8 |
| B9 | 3.5 | 31.4 | 3.5 | 0.1 |
| B11 | 3.4 | 14.2 | 3.5 | 0.6 |
| C5 | 3.3 | 13.9 | 3.5 | 0.6 |
| C6 | 2.6 | 13.2 | 3.5 | 0.6 |
| C7 | 2.5 | 21.2 | 3.5 | 0.1 |
| C10 | 2.8 | 25.8 | 3.5 | 0.7 |
| C12 | 2.9 | 23.3 | 3.5 | 1.0 |
| D8 | 3.2 | 17.3 | 3.5 | 0.1 |
| D12 | 2.3 | 41.0 | 3.5 | 0.1 |
| E8 | 2.9 | 11.2 | 3.5 | 0.4 |
| E12 | 3.5 | 19.4 | 3.5 | 0.7 |
| F6 | 2.7 | 13.8 | 3.5 | 0.6 |
| F10 | 2.6 | 18.3 | 3.5 | 1.0 |
| F12 | 2.0 | 9.5 | 3.5 | 0.5 |
| G5 | 3.1 | 10.7 | 3.5 | 0.4 |
| G10 | 2.6 | 21.5 | 3.5 | 0.7 |
| H6 | 3.2 | 12.7 | 3.5 | 0.6 |
| H7 | 2.1 | 11.5 | 3.5 | 1.0 |

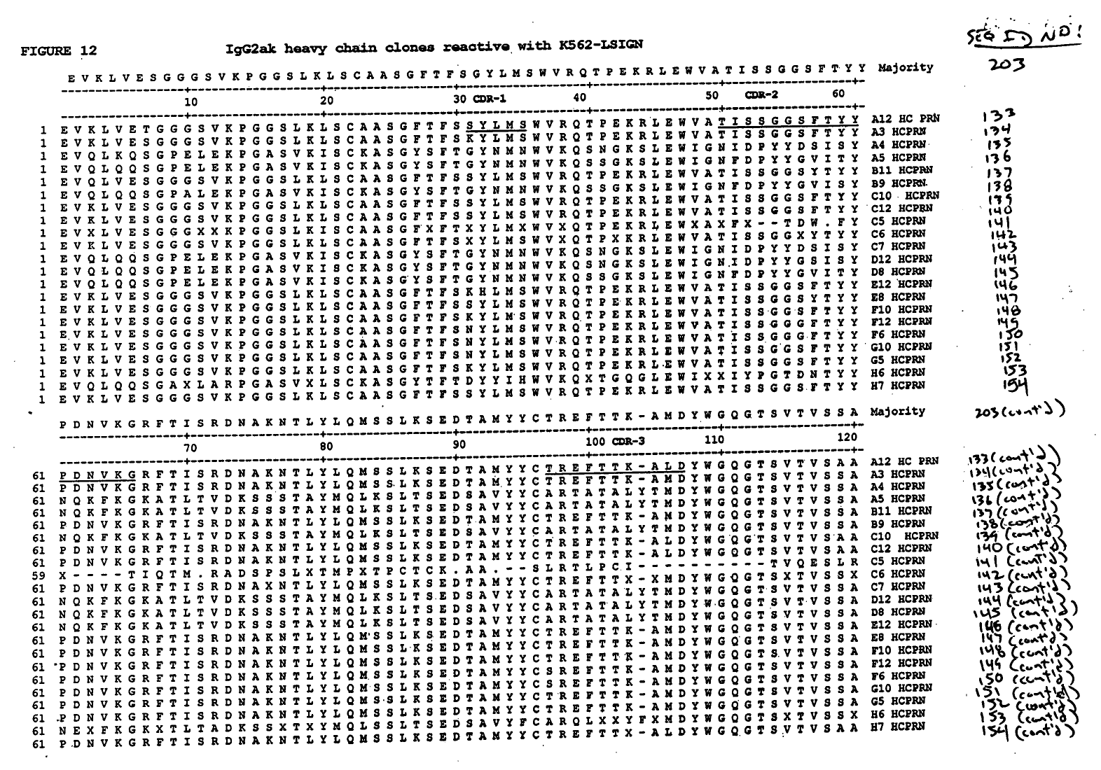

As set forth in FIG. 12, additional heavy chain clones that bind human DC-SIGNR were identified (SEQ. ID NOS: 133-154). These clones were found to have IgG2ak heavy chain CDR3 regions with one of the following amino acid sequences: T R E F T T K A L D (SEQ. ID NO: 155); T R E F T T K A M D (SEQ. ID NO: 156); A R T A T A L Y T M D (SEQ. ID NO: 157); L R T L P C I (SEQ. ID NO: 158); S R E F T T K A M D (SEQ. ID NO: 159); A R Q L X X Y F X M D (SEQ. ID NO: 160). The CDR2 regions of these clones that bound to human DC-SIGNR were found to have one of the following amino acid sequences: T I S S G G S F T Y Y P D N V K G (SEQ. ID NO: 161); N I D P Y Y D S I S Y N Q K F K G (SEQ. ID NO: 162); N F D P Y Y G V I T Y N Q K F K G (SEQ. ID NO: 163); T I S S G G S Y T Y Y P D N V K G (SEQ. ID NO: 164); X F X T D W . F Y X T (SEQ. ID NO: 165); N F D P Y Y G V I S Y N Q K F K G (SEQ. ID NO: 166); T I S S G G G F T Y Y P D N V K G (SEQ. ID NO: 167); X I Y P G T D N T Y Y N E X F K G (SEQ. ID NO: 168).

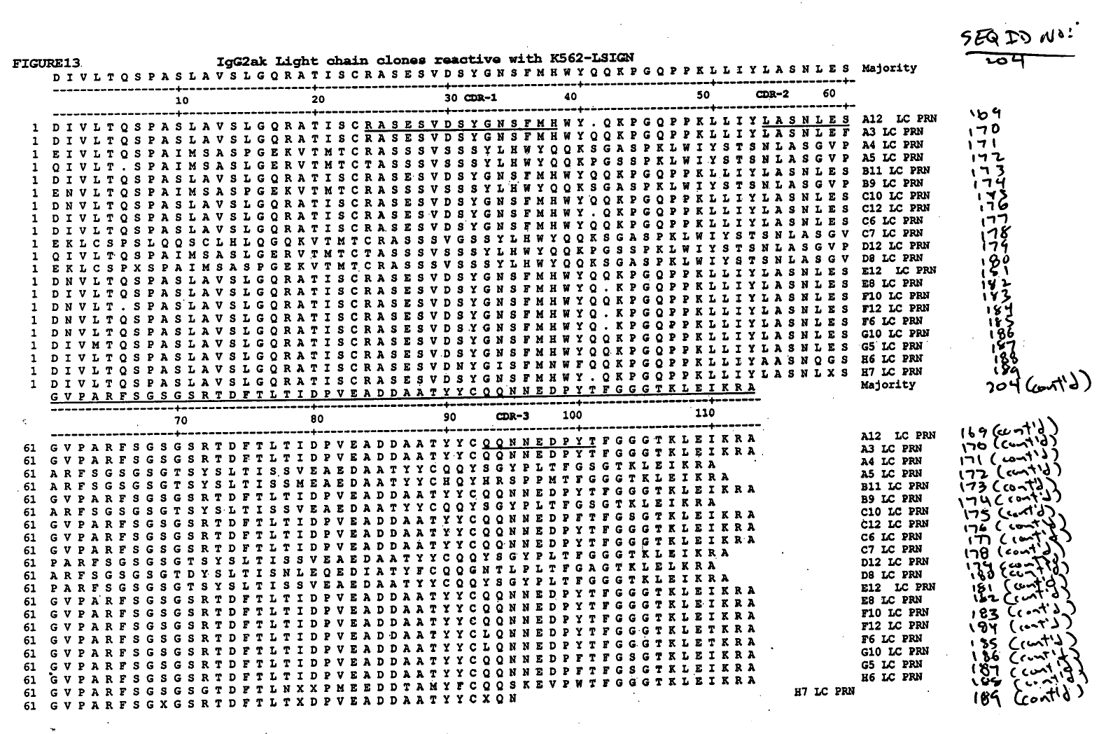

As set forth in FIG. 13, additional light chain clones that bind human DC-SIGNR were identified (SEQ. ID NOS: 169-189). These clones were found to have IgG2ak light chain CDR3 regions with one of the following amino acid sequences: Q Q N N E D P Y T (SEQ. ID NO: 190); S G Y P L T F G S (SEQ. ID NO: 191); H R S P P M T F G (SEQ. ID NO: 192); Q Q N N E D P F T (SEQ. ID NO: 193); Y S G Y P L T F G (SEQ. ID NO: 194); N T L P L T F G (SEQ. ID NO: 195); Q Q S K E V P W T (SEQ. ID NO: 196); L Q N N E D P Y T F (SEQ. ID NO: 197). The CDR2 regions of these clones that bound to human DC-SIGNR were found to have one of the following amino acid sequences: L A S N L E S (SEQ. ID NO: 198); L A S N L E F (SEQ. ID NO: 199); N L A S G V P (SEQ. ID NO: 200); N L A S G V (SEQ. ID NO: 201); A A S N Q G S (SEQ. ID NO: 202).

### EXAMPLE 11

### Selective Reactivity of Candidate Soluble L-SIGN Antibodies with L-SIGN Receptor But not DC-SIGN Receptor

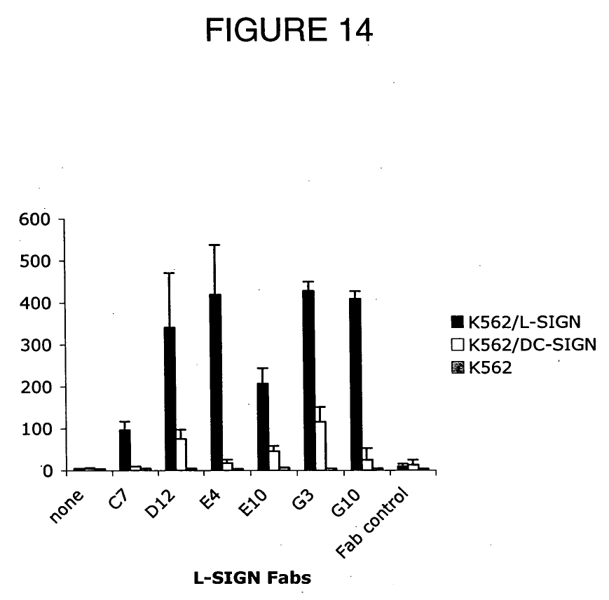

Based on phage-Fab screening, DNA sequencing (FIGS. 10 and 11), six antibodies (clone names C7, D12, E4, E10, G3, G10) having the best reactivity with cells expressing L-SIGN receptor (Table 4) were subcloned to remove the coat III protein. Each of these six candidate antibodies were expressed and purified as soluble Fab moieties and tested again for their ability to recognize the L-SIGN receptor but not DC-SIGN on cells. As shown in FIG. 14, all six antibodies exhibited very good binding with L-SIGN receptor, while three antibodies (D12, G3 and E10) also reacted with DC-SIGN but at substantially lower level. All antibodies (20 ug/ml) were incubated with 0.5×10⁶ cells (K562, K562/DC-SIGN, K562/L-SIGN) in FACS buffer (DPBS with 1% BSA, 0.1% Azide) for 1 hour at 4° C., washed with FACS buffer and incubated with phycoerythrin (PE) conjugated goat-anti mouse antibody (Jackson Immunoresearch, West Grove, Pa.), 1:50 dilution in FACS buffer at 4° C. for 30 minutes, washed and resuspended in 1% formaldehyde and analyzed on a BD FACSCalibur (Becton Dickinson, Mountain View, Calif.).

### EXAMPLE 12

### Relative Affinity and Epitope Characteristics of L-SIGN Antibodies

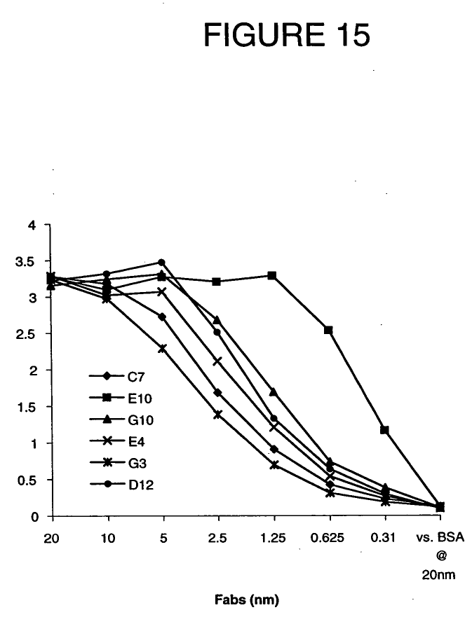

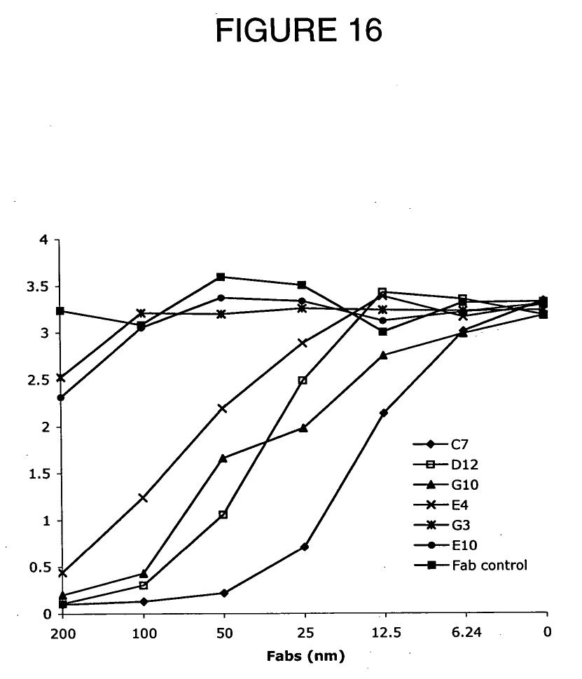

The six candidate antibodies were characterized further in terms of their affinity for the L-SIGN protein and the nature of epitope recognized. As shown in FIG. 15, while all antibodies react with the L-SIGN receptor with nanomolar affinities, clone E10 exhibited binding even in the picomolar range. Epitope specificity of different antibodies was characterized by competing out L-SIGN specific monoclonal antibody (mab162) binding to L-SIGNFc fusion protein in an ELISA. As shown in FIG. 16, four antibody clones, C7, D12, G10 and E4 competed out mab162 in a concentration dependent manner, while clone G3 and E10 did not compete. These data imply that clones C7, D12, G10 and E4 either share the same epitope or bind to overlapping epitopes as indicated by their subtle differences in the competition studies. To characterize the nature of epitope (conformational vs. linear; monomeric vs. multimeric) bound by the six antibodies, whole cell lysates of K562/L-SIGN cells prepared under denaturing and reducing conditions were separated by SDS-PAGE and the membrane probed with individual antibodies. Two antibodies (clone D12 and E10) recognized a protein band corresponding to the monomer, while clone G3 recognized a protein band corresponding to the trimeric form of the receptor. These data also indicate epitopes recognized by clone D12, E10 and G3 are linear, while epitopes recognized by antibody clones C7, E4 and G10 are conformational. The nature of epitopes recognized by L-SIGN fabs was delineated by western blotting studies. Briefly, one million K562/L-SIGN cells were lysed in 50 ul lysis buffer (150 mM NaCl; 25 mM Tris, pH 7.4; 2 mM EDTA, 1 mM sodium orthvanadate, 10 mM sodium fluoride; 1% Triton X-100, 0.5 mM PMSF, 10 μg/ml Aprotinin and 10 μg/ml Leupeptin). Lysis was achieved by gentle rotation at 4° C. for 20 minutes. Cell lysates were centrifuged (14,000×g, 10 minutes) to remove cell debris and boiled for 5 minutes in SDS sample buffer containing 1 mM DTT. Protein lysates were resolved on 4-15% SDS-PAGE gradient gels (Bio-Rad #116-1158), transferred to nitrocellulose membranes and then probed individually with L-SIGN specific fabs (1 μg/ml). Protein transfer was monitored with pre-stained molecular weight standards (Bio-Rad #161-0324). Immunoreactive bands were detected using HRP conjugated goat anti-mouse IgG (Bio-Rad #170-6516) by enhanced chemiluminescence (Super-signal West Pico kit, Pierce, Rockford, Ill.).

### EXAMPLE 13

### L-SIGN Antibodies Undergo Internalization Upon Binding to the Receptor on Human Liver Non-Parenchymal Cells

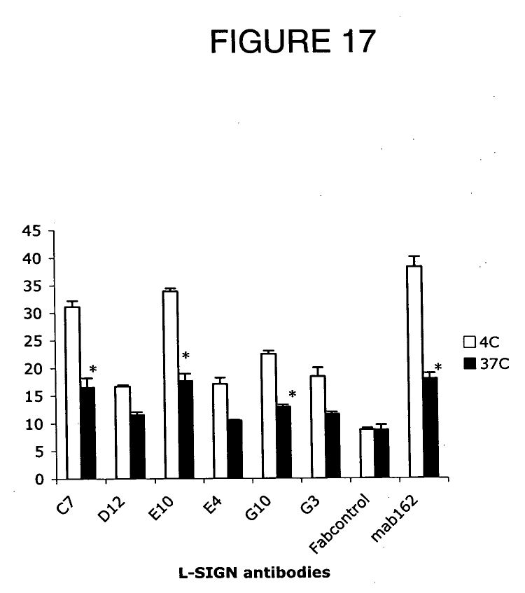

To deliver antigens into L-SIGN expressing sinusoidal endothelial cells (LSECs), which are known to cause immune tolerance, the internalizing potential of the antibody was assessed by flow cytometry and confocal microscopy. Freshly isolated human liver non-parenchymal cells were incubated with the six candidate antibodies. As shown in FIG. 17, three antibodies (C7, E10 and G10) exhibited over forty-percent internalization in two hours. This level of internalization was found to be statistically significant (p<0.05). Furthermore, antibody internalization was also confirmed directly by confocal microscopy studies. All six antibodies were found inside K562/L-SIGN cells following a ninety minute incubation, while no uptake was observed with K562/DC-SIGN cells used as control in this study. These studies demonstrate that while all of the L-SIGN antibodies are capable of being internalized, three antibodies (clones C7, E10 and G10) internalize most efficiently.

Antibody Internalization Assay

The assay was done as described by Takahara K et al (International Immunology, 2004). Briefly, 0.5×10⁶ cells (fresh human LSECs or K562/L-SIGN) were incubated with 20 μg/ml of L-SIGN fabs for 30 minutes at 4° C. in DPBS/1% BSA, washed off unbound antibody and then incubated at 37° C. for an additional 2 hours to enable internalization. Duplicate samples kept at 4° C. in DPBS/1% BSA/0.1% Azide served as controls. At the end of the incubation period, cells were incubated with phycoerythrin (PE) conjugated goat-anti mouse antibody for 30 minutes at 4° C. in DPBS/1% BSA/0.1% Azide, washed, fixed in 1% formaldehyde and analyzed on FACSCalibur (Becton Dickinson).

Confocal Microscopy

10⁵ K562 transfectants were incubated with 10 μg/ml of the various antibodies for 90 minutes at 37° C. in RPMI 1640 (GIBCO, Life Technologies, Breda, The Netherlands) supplemented with 10% fetal calf serum. Cells were then washed with PBS, fixed in PBS/4% paraformaldehyde, washed again and adhered to poly-L-lysin coated coverslips (20 minutes at room temperature). Cells were incubated with blocking buffer, PBS/3% BSA (Sigma Chemical Co., St. Louis, Mo.)/10 mM glycine (Merck, Darmstadt, Germany)/0.1% saponin (Riedel de Haen, Seelze, Germany), for 1 hour at room temperature. Subsequently, cells were incubated with 10 μg/ml rabbit anti-human CD55 (Santa Cruz Biotechnology, Santa Cruz, Calif.) for 1 hour at room temperature, washed with blocking buffer and incubated with 10 μg/ml goat anti-mouse IgG Alexa 647 (Molecular Probes, Leiden, The Netherlands) and 10 μg/ml goat anti-rabbit IgG Alexa 488 (Molecular Probes) in blocking buffer for 1 hour at room temperature. Cells were then washed with blocking buffer, with PBS and finally with 50 mM Tris-HCl. Finally, coverslips were mounted onto glass-slides with Mowiol (Calbiochem, Omnilabo International, Breda, The Netherlands).

### EXAMPLE 14

### L-SIGN Antibodies Block Binding of Ligand to the Receptor

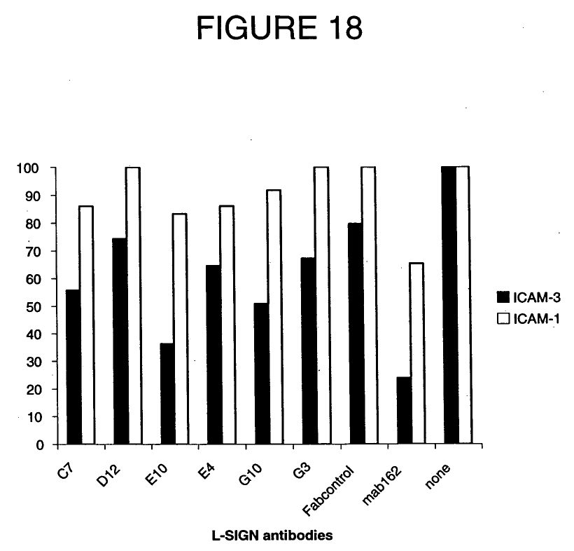

Several viruses e.g., Ebola, SARS, HIV and HCV have been shown to utilize L-SIGN receptor to gain entry into cells. Both the envelope glycoproteins on the surface of the viruses and ICAM-3 on the surface of host T-cells are known to interact with the lectin-binding domain of L-SIGN. To determine if the present antibodies are capable of blocking the L-SIGN receptor and ligand interaction, ligand coated fluorescent bead-blocking assay was performed. As shown in FIG. 18, three antibodies (C7, G10 and E10) that internalized most efficiently also blocked the binding of ICAM-3 to L-SIGN receptor the best (>40% inhibition was observed) without affecting the binding of ICAM-1 coated beads used as control in the assay. (See FIG. 18.)

Fluorescent Beads Adhesion Assay for Ligand Blocking.

Carboxylate-modified TransFluorSpheres (488/645 nm, 1.0 μm; Molecular Probes) were coated with ICAM-3 Fc protein (R & D systems, Minneapolis, Minn.) as was previously described for ICAM-1 beads (⁵Geijtenbeek et al, 1999.). Briefly, streptavidin-coated beads were incubated with biotinylated goat-antihuman anti-Fc Fab2 fragments for 2 hours at 37° C. in PBS, 0.5% BSA. The beads were washed and incubated with human IgG1 Fc fused ICAM-3 Fc (250 ng/mL) overnight at 4° C. For adhesion to ICAM-3 beads, K562/L-SIGN cells were resuspended in Tris-sodium-BSA (20 mmol/L Tris-HCl, pH 8.0, 150 mmol/L NaCl, 1 mmol/L CaCl2, 2 mmol/L MgCl2, 0.5% BSA (wt/vol); 5 Å˜106 cells/mL). Fifty thousand cells were preincubated with or without L-SIGN fabs (20 μg/mL) for 10 minutes at room temperature in a 96-well V-shaped-bottom plate. The ligand-coated beads (20 beads/cell) were added and the suspension was incubated for 30 minutes at 37° C. After washing, the cells were resuspended in Tris-sodium-BSA buffer. ICAM-3-mediated adhesion of K562/LSIGN cells was measured by flow cytometry using the FACSCalibur (Becton Dickinson). The percentage of cells bound to ICAM-3 in absence of antibodies was set at 100 and the decrease in binding in presence of L-SIGN antibodies expressed as ligand blocking.

### EXAMPLE 15

### A Summary of Biological Activities Mediated by L-SIGN Specific Antibodies

Based on the foregoing studies (see Table 7 below), at least three antibody clones C7, G10 and E10 have the potential for therapeutic application in autoimmunity and infectious disease.

TABLE 7

| | Receptor | Internalization | Internalization by | ICAM-3 |
|---|---|---|---|---|
| Clone | Specificity | by LSECs (%) | K562/LSIGN (%) | blocking (%) |
| D12 | L-SIGN/DC- | 31 | 25 | 26 |
| | SIGN^{a} | | | |
| C7 | L-SIGN | 47 | 26 | 44 |
| E4 | L-SIGN | 38 | 27 | 35 |
| G10 | L-SIGN | 42 | 32 | 45 |
| E10 | L-SIGN/DC- | 48 | 27 | 64 |
| | SIGN^{a} | | | |
| G3 | L-SIGN/DC- | 37 | 46 | 33 |
| | SIGN^{a} | | | |

^{a}Reactivity with DC-SIGN is significantly lower (<25%) than with L-SIGN

### EXAMPLE 16

### Selecting Antibodies Capable of Blocking Virus Binding

The selection of antibodies capable of blocking viral entry is accomplished by a fluorescent bead assay as described by ⁵Geijtenbeek et al. (1999). Briefly, fifty thousand K562/L-SIGN transfected cells are preincubated with or without L-SIGN Fabs (20 μg/mL) for 10 minutes at room temperature in a 96-well V-shaped bottom plate. Fluorescent beads (20 beads/cell) coated with viral envelope proteins, e.g., HCV E1/E2 or HIV gp120 are added and the suspension incubated for an additional 30 minutes at 37° C. After washing, the cells are resuspended in Tris-sodium-BSA buffer. The extent of blocking by antibodies of virus coated beads to K562/L-SIGN cells is measured using a FACSCalibur (Becton Dickinson). The percentage of cells bound to the virus beads (negative control) in the absence of antibodies is set at 100 and the decrease in binding in the presence of L-SIGN antibodies expressed as % blocking.

### EXAMPLE 17

### Blocking Viral Entry

After selecting antibodies capable of blocking virus binding to the receptor in Example 16 above, the capacity of these antibodies to block viral entry is tested. L-SIGN transfected K-562 cells are preincubated with antibodies for 30 minutes before adding reporter viruses expressing envelope proteins of interest or when feasible serum from virus+ or virus− donors. After 1 hour of incubation at 37° C., cells are washed 5 times with phosphate buffered saline and viral RNA is extracted from the cells using Qiagen's viral RNA mini spin kit. Viral RNA thus obtained is amplified by RT-PCR following the procedures of Gardner et al. (2003) and a southern blot is performed.

### EXAMPLE 18

### Preventing Viral Transmission

To test whether the antibodies identified in Example 16 can prevent transfer of a virus from receptor positive endothelial cells to either human T-cells or liver cells, K562/L-SIGN cells or freshly isolated human liver sinusoidal endothelial cells (L-SIGN+) or dendritic cells (DC-SIGN+) are incubated with the antibodies of Example 16 for 30 minutes before adding luciferase or green fluorescent protein reporter viruses expressing envelope proteins of interest, e.g., HCV-E2, HIV gp120, Ebola (Alvarez et al. 2002) or Sindbis (Klimstra et al. 2003). After washing with culture medium, the cells are co-cultured with T-cells (C8166) or human liver cells (Huh-7). Reporter virus transmission is assessed either by measuring luciferase activity (relative light units) in target cell lysates or by flow cytometric analysis of GFP positive target cells in combination with suitable surface marker double staining on target cells (e.g., CD3 on T-cells).

### EXAMPLE 19

### Assessing the Role of Antibodies in Blocking Infection from Mycobacterium tuberculosis

Mannosylated lipoarabinomannan (ManLAM), a carbohydrate rich structure present on the surface of M. tuberculosis has been reported to interact with both DC-SIGN (⁴Geijtenbeek et al. 2003) and L-SIGN (Koppel et al. 2004). High antibody titers against ManLAM are observed in people with active tuberculosis and have been shown to reduce bacterial loads in passive protection experiments (Hamasur et al. 2004). Mycobacterial binding and infection are inhibited using L-SIGN antibodies or L-SIGN peptide mimics capable of binding to ManLAM with high affinities. Strains of bacterium, e.g., M. bovis and M. tuberculosis are labeled with fluorescein isothiocyanate (FITC) as detailed in ⁴Geijtenbeek et al. (2003). K562/L-SIGN cells are incubated with FITC conjugated bacteria at a ratio of 1 to 20 in the presence or absence of antibodies of L-SIGN peptide mimics (50 μg/ml). The extent of blocking (reduction in fluorescence) by the antidotes or mimics is determined by flow cytometry analysis.

### EXAMPLE 20

### Treatment of Transplant Patients with HCV Infected Liver

If virus transmission is prevented, the antibodies can be used in a transplant setting in which donors potentially have HCV infections. To test this, mildly HCV-infected human donor liver are transplanted into immunodeficient mice such as NOD/SCID alongside with injection of primary blood lymphocytes from a healthy, HLA matched human donor. Mice are treated with antibodies over a period of one to 6 months. One to six months after transplantation, the mice are sacrificed, and the extent of HCV infection in the liver is assessed. Also, T cells are examined for infection with virus by PCR.

### EXAMPLE 21

### Identification of Cancer Types Expressing L-SIGN

Malignant tissues and matching normal tissues are collected and fixed in para-formaldehyde or snap-frozen in OCT. Sections are prepared using a microtome, and the sections are stained for the presence of L-SIGN using L-SIGN antibodies either directly conjugated to a suitable fluorochrome, such as FITC, or using a secondary fluorochrome-conjugated anti-mouse IgG. Staining results for malignant tissue that are significantly stronger than staining of normal tissue are indicative of cancer types expressing L-SIGN.

Commercially available cell lines for those cancer types expressing L-SIGN are then obtained and the presence of L-SIGN is evaluated by FACS analysis. Briefly, one million cells are incubated with 1 μg of anti-L-SIGN antibody in PBS containing 1% BSA and 0.1% NaN₃. After 30 minutes incubation, the cells are washed and incubated with fluorochrome-conjugated anti-mouse IgG for another 30 minutes before analysis with a FACSCalibur (Becton Dickinson).

### EXAMPLE 22

### Therapeutic Use of Anti-L-SIGN Antibodies

Direct Cell Killing

Once an L-SIGN expressing tumor type has been identified as in Example 21 above, anti-L-SIGN antibodies are tested for their capacity to induce ADCC or CDC in vitro. For evaluating ADCC, target cells (tumor cells) are first labeled with ⁵¹Cr. Antibodies to L-SIGN are then added at concentrations between 1-50 μg and target cell lysis by PBMC is determined after 4 hours at effector to target ratios of 1:10-1:100. For evaluation of CDC, tumor cells are incubated with human complement and L-SIGN antibodies. Cell killing is assessed by FACS analysis after addition of propidium iodide, a reagent that can only enter dead, but not live, cells. For antibodies that do not induce ADCC or CDC, any radiolabel or toxic reagent will remain conjugated to the antibody.

The capacity of either the naked or conjugated anti-L-SIGN antibodies to halt tumor growth is assessed in xenograft models. Briefly, tumor cells expressing L-SIGN are injected subcutaneously, intraperitoneally or intravenously. Animals are treated with either control or anti-L-SIGN antibodies. Tumor growth is measured by size for subcutaneous treatment or survival time for intraperitoneal or intravenous treatment. If tumor growth in the anti-L-SIGN treated groups is reduced by more than 30% compared to the control group, the antibodies may be utilized as a cancer therapeutic.

Blocking of Negative Regulatory Interaction of Cancer Cells with the Immune System

Antibodies blocking the interaction of L-SIGN with immune cells are identified using the fluorescent bead assay as described in example 14. The antibodies are evaluated for their therapeutic usefulness with regard to allowing the immune system to eradicate cancer cells by preventing negative regulation of the immune system through L-SIGN expressing cancer cells.

L-SIGN expressing tumor cells are implanted subcutaneously, intraperitoneally or intravenously into immune-deficient mice such as NOD/SCID. Mice will also receive 2 million PBMC's from healthy donors (or any number of PBMC's not sufficient to reject tumors by themselves). Tumor growth in the presence or absence of anti L-SIGN antibody is compared with control antibody. Tumor growth is measured by size for subcutaneous treatment or survival time for systemic tumors. If tumor growth in the anti-L-SIGN treated groups is reduced by more than 30% compared to the control group, the antibodies may be utilized as a cancer therapeutic.

### EXAMPLE 23

### Use of L-SIGN Antibodies to Isolate L-SIGN Expressing Cells

Fresh human non-parenchymal liver cells are obtained from commercial sources (e.g. CellzDirect, Tucson, Ariz.). Non-parenchymal liver cells contain a mixture of sinusoidal endothelial cells, red blood cells, Kupffer cells and other minor cell populations. To isolate liver sinusoidal cells, red blood cells are lysed using ammonium chloride. Dead cells are removed using commercially available dead cell removal kits (Miltenyi Biotech, Germany). Cells are counted and labeled with 0.1 μg/million cells of anti-L-SIGN antibody. After washing the cells, they are resuspended in MACS buffer (Miltenyi) and the cells of interest are isolated using anti-mouse IgG-conjugated beads according to the manufacturer's instructions (Miltenyi). The quality of the isolation is monitored by FACS analysis using a panel of antibodies against molecules such as CD54, mannose receptor, LYVE-1, CD40, asialoglycoprotein and others.

The following references are incorporated herein in their entirety by this reference:
Alvarez, C. P., F. Lasala, J. Carrillo, O. Muniz, A. L. Corbi, and R. Delgado. 2002. C-type lectins DC-SIGN and L-SIGN mediate cellular entry by Ebola virus in cis and in trans. J Virol 76:6841.
Aspord C, Thivolet C (2002) Nasal administration of CTB-insulin induces active toleranceagainst autoimmune diabetes in non-obese diabetic (NOD) mice. Clin Exp Immunol 130:
Atkinson M A, Kaufman D L, Campbell L, Gibbs K A, Shah S C, Bu D F, Erlander M G, Tobin A J, Maclaren N K (1992) Response of peripheral-blood mononuclear cells to glutamate decarboxylase in insulin-dependent diabetes. Lancet 339: 458-459.
Atkinson M A, Leiter E H (1999) The NOD mouse model of type I diabetes: As good as it gets? Nat Med 6: 601-604.
Atkinson M A, MacLaren N K, Luchetta R (1990) Insulitis and diabetes in NOD mice reduced by prophylactic insulin therapy. Diabetes 39: 956-960.
Bach J F (2001 ) Immunotherapy of insulin-dependent diabetes mellitus. Curr Opin Immunol 13: 601-605.
Baekkeskov S, Aanstoot H J, Christgau S, Reetz A, Lolimena M, Cascalho F, Folli F, Richter-Oelsen H, Decamilli P (1990) Identification of the 64 K autoantigen in insulin-dependent diabetes as the GABA-synthesizing enzyme glutamic acid decarboxylase. Nature 347: 151-156.
Barrat F J, Cua D J, Boonstra A, Richards D F, Crain C, Savelkouf H F, de Waal-Malefyt R, Coffman R L, Hawrylowicz C M, O'Garra A (2002) In vitro generation of interleukin 10-producing regulatory CD4(+) T cells is induced by immunosuppressive drugs and inhibited by T helper type i (ThI) and Th2-inducing cytokines. J Exp Med 195: 603-616.
Bashirova A A, Geijtenbeek T B H, van Duijnhoven G C F et al (2001) A dendritic cell-specific intercellular adhesion molecule 3-grabbing non-integrin (DC-SIGN) related protein is highly expressed on human liver sinusoidal endothelial cells and promotes HIV-1 infection. J Exp Med 193: 671-678.
Bellmann K, Kolb H, Rastegar S Jee P, Scott F W (1998) Potential risk of oral insulin with adjuvant for the prevention of type I diabetes: a protocol effective in NOD mice may exacerbate disease in BB rats. Diabetologia 41: 844-847.
Bercovici N, Heurtier A, Vizler C, Pardigon N, Cambouris C, Desreumaux P, Liblau R (2000) Systemic administration of agonist peptide blocks the progression of spontaneous CD8-mediated autoimmune diabetes intrasgenic mice without bystander damage. J Immunol 165:202-210.
Blana E, Carbone F R, Allison H, Miller J F, Heath W R (1996) Induction of autoimmune diabetes by oral administration of autoantigen. Science 274: 1707-1709.
Boitard C, Yasunami R, Dardenne M, Bach J F (1989) T cell mediated inhibition of the transfer of autoimmune diabetes in NOD mice. J Exp Med 169: 1669-1680.
Calne R (1969) Induction of immunological tolerance by porcine liver allografts. Nature 223:427-476.
Cameron M J, Arreaza G A, Zucker P, Chensue S W, Strieter R M, Chakrabarti S, Delovitch T L (1997) IL-4 prevents insulitis and insulin-dependent diabetes mellitus in nonobese diabeticmice by potentiation of regulatory T helper-2 cell function. J Immunol 159: 4686-4692.
Chaillous L, Lefevre H, Thivolet C, Boitard C, Lahlou N, Atlan Gepner C, Bouhanick B, Mognet A, Nicolino M, Carel J, Lecomte P, Marechaud R, Bougneres P, Charbonnel B, Sai P (2000) Oral insulin administration and residual beta-cell function in recent-onset type 1 diabetes: a multicentre randomized controlled trial. Lancet 356: 545-549.
Chatenoud L, Primo J, Bach J F (1997) CD3 antibody-induced self tolerance in overtly diabetic NOD mice. J Immunol 158:2947-2954.
Chatenoud L, Thervet E, Promo J, Bach J F (1994) Anti-CD3 antibody induces long-term remission of overt autoimmunity in nonobese diabetic mice. Proc Natl Acad Sci USA 89:3434-3438.
Cormier, E. G., R. J. Durso, F. Tsamis, L. Boussemart, C. Manix, W. C. Olson, J. P. Gardner, and T. Dragic. 2004. L-SIGN (CD209L) and DC-SIGN (CD209) mediate transinfection of liver cells by hepatitis C virus. Proc Natl Acad Sci USA.
Daniel D, Gill R G, Schloot N, Wegmann D (1995) Epitope specificity, cytokine production profile and diabetogenic activity of insulin-specific T cell clones isolated from NOD mice. Eur. J Immunol 25: 1056-1062.
Dhodapkar M V, Steinman R M (2001) Antigen-specific inhibition of effector T cell function in humans after injection of immature dendritic cells. J Exp Med 193:233-238.
Dhodapkar M V, Steinman R M (2002) Antigen-bearing immature dendritic cells induce peptide-specific CD8(+) regulatory T cells in vivo in humans. Blood 100:174-177.
Diabetes Prevention Trial-Type I (DPT) Study Group (2002): Effects of parental insulin in relatives at high risk of type 1 diabetes mellitus. N Eng J Med 346: 1685-1691.
Elias D, Cohen I R (1994) Peptide therapy for diabetes in NOD mice. Lancet 343: 704-706.
Elias D, Meilin A, Ablamuntis V, Birk O S, Carmi P, Konen-Waisman S, Cohen I R (1997) Hsp6O peptide therapy of NOD mouse diabetes induces a Th2 cytokine burst and down regulates autoimmunity to various beta cell antigens. Diabetes 46: 758-764.
Elias D, Reshef T, Birk O S, Van der Zee R, Walker M D, Cohen I R (1991) Vaccination against autoimmune mouse diabetes with a T cell epitope of the human 65 kDa heat shock protein. Proc Natl Acad Sci USA 88: 3088-3091.
Engering A, Geijtenbeek T B H, van Vliet S J et al (2002) The dendritic cell-specific adhesion receptor DC-SIGN internalizes antigen for presentation to T cells. J Immunol 168: 2118-2126.
Feutren G, Papoz L, Assan R et al (1988) Cyclosporin increases the rate and length of remissions in insulin-dependent diabetes of recent onset. Results of a multicenter double-blind trail. Lancet ii:I19-124.
Gardner, J. P., R. J. Durso, R. R. Arrigale, G. P. Donovan, P. J. Maddon, T. Dragic, and W. C. Olson. 2003. L-SIGN (CD 209L) is a liver-specific capture receptor for hepatitis C virus. Proc Natl Acad Sci USA 100:4498.
¹Geijtenbeek T B H, Groot P C, Nolte M A, van Vliet S J, Gangaram-Panday S T, van Duijnhoven G C F, Kraal G, van Oosterhout A J M, van Kooyk Y (2002) Marginal zone macrophages express a murine homologue of DC-SIGN that captures blood-borne antigens in vivo. Blood 100: 2908-2916.
²Geijtenbeek T B H, Krooshoop D J E B, Bleijs D A et al (2000) DC-SIGN-ICAM-2 interaction mediates dendritic cell trafficking. Nat Immunol 1: 252-257.
³Geijtenbeek T B H, Torensma R, van Vliet S J, van Duijnhoven G C F, Adema G J, van Kooyk Y, Figdor C G (2000) Identification of DC-SIGN, a novel dendritic cell-specific CAM3 receptor that supports primary immune responses. Cell 100: 575-585.
⁴Geijtenbeek, T. B., S. J. Van Vliet, E. A. Koppel, M. Sanchez-Hernandez, C. M. Vandenbroucke-Grauls, B. Appelmelk, and Y. Van Kooyk. 2003. Mycobacteria target DC-SIGN to suppress dendritic cell function. J Exp Med 197:7.
⁵Geijtenbeek, T. B., Y. van Kooyk, S. J. van Vliet, M. H. Renes, R. A. Raymakers, and C. G. Figdor. 1999. High frequency of adhesion defects in B-lineage acute lymphoblastic leukemia. Blood 94:754.
Halary, F., A. Amara, H. Lortat-Jacob, M. Messerle, T. Delaunay, C. Houles, F. Fieschi, F. Arenzana-Seisdedos, J. F. Moreau, and J. Dechanet-Merville. 2002. Human cytomegalovirus binding to DC-SIGN is required for dendritic cell infection and target cell trans-infection. Immunity 17:653.
Hamasur, B., M. Haile, A. Pawlowski, U. Schroder, G. Kallenius, and S. B. Svenson. 2004. A mycobacterial lipoarabinomannan specific monoclonal antibody and its F(ab′) fragment prolong survival of mice infected with Mycobacterium tuberculosis. Clin Exp Immunol 138:30.
Hawiger D, Inaba K, Dorsett Y, Guo M, Mahnke K, Rivera M, Ravetch J V, Steinman R M, Nussenzweig M C (2001 ) Dendritic cells induce peripheral T cell unresponsiveness under steady state conditions in vivo. J Exp Med 194:769-779.
Heath W R, Carbone F R (2001 ) Cross-presentation, dendritic cells, tolerance and immunity. Annu Rev Immunol 19: 47-64.
Herold K C, Hagopian W, Auger J A, Poumian-Ruiz E, Taylor L, Donaldson D, Gitelman S E, Harlan D, Xu D, Zivin R A, Bluestone J A (2002) Anti-CD3 monoclonal antibody in new-onset type I diabetes mellitus. N Eng J Med 346: 1692-1698.
Homann D, Holz A, Bot A, Coon B, Wolfe T, Petersen J, Dyrberg T P, Grusby M J, von Herrath M G (1999) Autoreactive CD4+ T cells protect from autoimmune diabetes via bystander suppression using the IL-4/Stat6 pathway. Immunity 11: 463-472.
Honeyman M C, Cram D S, Harrison L C (1993) Glutamic acid decarboxyase 67-reactive T cells: a marker of insulin-dependent diabetes. J. Exp. Med. 177:335-340.
Huges S, Mougneau E Ferlin W, Jeske D, Hofman P, Homann D, Beaudoin L, Schrike C, von Herrath M, Lehuen A et al (2002) Tolerance to islet antigens and prevention from diabetes induced by limited apoptosis of pancreatic beta cells. Immunity 16:169-181.
Hurlbert M S, Stallard J F, Furlanetto R W (2001 ) Symposium summary, Experimental Biology. J Investig Med 49:554-558.
Hutchings P R, Cooke A (1990) The transfer of autoimmune diabetes in NOD mice can be inhibited or accelerated by distinct cell populations present in normal splenocytes taken from young males. J Autoimmun 3: 175-185.
Jeffers, S. A., S. M. Tusell, L. Gillim-Ross, E. M. Hemmila, J. E. Achenbach, G. J. Babcock, W. D. Thomas, Jr., L. B. Thackray, M. D. Young, R. J. Mason, D. M. Ambrosino, D. E. Wentworth, J. C. Demartini, and K. V. Holmes. 2004. CD209L (L-SIGN) is a receptor for severe acute respiratory syndrome coronavirus. Proc Natl Acad Sci USA 101:15748.
Jonuleit H, Schmitt E, Schuler G, Knop J, Enk A H (2000) Induction of interleukin 10-producing, nonproliferating CD4 T cells with regulatory properties by repetitive stimulation with allogeneic immature human dendritic cells. J Exp Med 192: 1213-1222.
Kamei T, Callery M P, Flye M W (1990) Pretransplant portal venous administration of donor antigen and portal venous allograft drainage synergistically prolong rat cardiac allograft survival. Surgery 108: 415-421.
Kaufman D L, Claire-Salzler M, Tian J, Forsthuber T, Ting G S P, Robinson P, Atkinson M A, Sercarz E E, Tobin A J, Lehmann P V (1993) Spontaneous loss of T-cell tolerance to glutamic acid decarboxylase in murine insulin-dependent diabetes. Nature 366:69-72.
Kaufman D L, Erlander M G, Clare-Salzer M., Atkinson M A, Maclaren N K, Tobin A J (1992) Autoimmunity to two forms of glutamate decarboxylase in insulin-dependent diabetes mellitus. J. Clin. Invest. 89:283-292.
King C, Mueller H R, Malo C M, Murali-Krishna K, Ahmed R, King E, Sarvetnick N (2002) Interleukin-4 acts at the locus of the antigen-presenting dendritic cell to counter-regulate cytotoxic CD8+ T cell responses. Nat Med 7:206-214.
Klimstra, W. B., E. M. Nangle, M. S. Smith, A. D. Yurochko, and K. D. Ryman. 2003. DC-SIGN and L-SIGN can act as attachment receptors for alphaviruses and distinguish between mosquito cell- and mammalian cell-derived viruses. J Virol 77:12022.
Klugewitz K, Blumenthal-Barby F, Schrage A, Knolle P A, Hamann A, Crispe I N (2002) Intramodulatory effects of the liver: deletion of activated CD4+ cells and suppression of IFN-γ producing cells after intravenous protein immunization. J Immunol 169: 2407-2413.
¹Knolle P, Gerken G (2000) Local regulation of the immune response in the liver. Immunol Rev 174: 21-34.
²Knolle P A, Germann T, Treichel U, Uhrig A, Schmitt E, Hegenbarth S, Lohse A W, Gerken G (1999) Endotoxin down-regulates T cell activation by antigen-presenting liver sinusoidal endothelial cells. J Immunol 162: 1401-1407.
³Knolle P A, Schmitt E, Jin S, Germann T, Duchmann R, Hegenbarth S, Gerken G, Lohse A W (1999) Induction of cytokine production in naive CD4 T cells by antigen-presenting murine liver sinusoidal endothelial cells but failure to induce differentiation toward Th1 cells. Gastroenterology 1 16: 1428-1440.
Knook D L, Sleyster E C H (1976) Separation of Kupffer and endothelial cells of the rat liver by centrifugal elutriation. Exp Cell Res 99: 444-449.
Koppel, E. A., I. S. Ludwig, M. S. Hernandez, T. L. Lowary, R. R. Gadikota, A. B. Tuzikov, C. M. Vandenbroucke-Grauls, Y. van Kooyk, B. J. Appelmelk, and T. B. Geijtenbeek. 2004. Identification of the mycobacterial carbohydrate structure that binds the C-type lectins DC-SIGN, L-SIGN and SIGNR1. Immunobiology 209:117.
Kretz-Rommel A, Boelsterli U A (1995) Cytotoxic activity of T cells and non-T cells from diclofenac-immunized mice against cultured syngeneic hepatocytes exposed to diclofenca. Hepatology 22: 213-222.
Kretz-Rommel A, Rubin R L (1997) A metabolite of the lupus-inducing drug procainamide prevents anergy induction in T cell clones. J Immunol 158: 4465-4470.
Kurts C, Sutherland R M, Davey G, Li M, Lew A M, Blanas E, Carbone F R, Miller J F A P, Heath W R (1999) CD8 T cell ignorance or tolerance to islet antigens depends on antigen dose. Proc Natl Acad Sci USA 96: 12703-12707.
Lafaille M A C D and Lafaille J J (2002) CD4+ regulatory T cells in autoimmunity and allergy. Curr Opin Immunol 14:771-778.
Limmer A, Ohl J, Kurts C, Ljunggren H G, Reiss Y, Groettrup M, Momburg F, Arnold B, Knolle P A (2000) Efficient presentation of exogenous antigen by liver endothelial cells to CD8+ T cells results in antigen-specific T-cell tolerance. Nat Med 6: 1348-1354.
Lohman T, Leslie R G D, Londei M (1996) T cell clones to epitopes of glutamic acid decarboxylase 65 raised from normal subjects and patients with insulin-dependent diabetes. J Autoimmun 9: 385-389.
Lohse A W, Knolle P A, Bilo K, Uhrig A, Waldmann C, Ibe M, Schmitt E, Gerken G, Meyer Zum Buschenfelde K H (1996) Antigen-presenting function and B7 expression of murine sinusoidal endothelial cells and Kupffer cells. Gastroenterology 110: 1175-1181.
Ma S W, Zhao D L, Yin Z Q, Mukherjee R, Singh B, Qin H Y, Stiller C R, Jevnikar A M (1997) Transgenic plants expressing autoantigens fed to mice to induce oral immune tolerance. Nat Med 3: 793-796.
Mahnke K, Guo M, Lee S, Sepulveda H, Swain S L, Nussenzweig M, Steinman R M (2000) The dendritic cell receptor for endocytosis, DEC-205, can recycle and enhance antigen presentation via major histocompatiblity complex class II-positive lysosomal compartments. J Cell Biol 151: 673-685.
Miyazaki A et al (1985) Predominance of T lymphocytes in pancreatic islets and spleen of pre-diabetic non-obese (NOD) mice: a longitudinal study. Clin Exp Immunol 60:622-630.
Notkins A L, Lernmark A (2001) Autoimmune type I diabetes: resolved and unresolved issues. J Clin Invest 108: 1247-1252.
O'Reilly L A, Hutchings P R, Crocker P R, Simpson E, Lund T, Kiousses D, Takei F, Baird J, Cooke A (1991) Characterization of pancreatic islet cell infiltrates in NOD mice: effect of cell transfer and transgene expression. Eur J Immunol 21:1171-1180.
Pakkala S V, Kurrer M O, Katz J D (1997) T helper 2 (Th2) cells induce acute pancreatitis and diabetes in immune-compromised nonobese diabetic (NOD) mice. J Exp Med 186:299-306.
Park C G, Takahara K, Umemoto E et al (2001) Five mouse homologues of the human dendritic cell C-type lectin, DC-SIGN. Int Immunol 13: 1283-1290.
Parving H H, Tarnow L, Nielsen F, Rossing P, Mandrup-Poulsen T, Osterby R, Nerup J (1999) Cyclosporine nephrotoxicity in type 1 diabetic patients. A 7-year follow-up study. Diabetes Care 22: 478-483.
¹Pohlmann S, Soilleux E J, Baribaud F et al (2001) DC-SIGNR, a DC-SIGN homologue expressed in endothelial cells, binds to human and simian immunodeficiency viruses and activates infection in trans. Proc Natl Acad Sci USA 98: 2670-2675.
²Pohlmann, S., G. J. Leslie, T. G. Edwards, T. Macfarlan, J. D. Reeves, K. Hiebenthal-Millow, F. Kirchhoff, F. Baribaud, and R. W. Doms. 2001. DC-SIGN interactions with human immunodeficiency virus: virus binding and transfer are dissociable functions. J Virol 75:10523.
Poul M A, Becerril B, Nielsen U B, Morisson P, Marks J D (2000) Selection of tumor specific internalizing human antibodies from phage libraries. J Mol Biol 301: 1149-1161.
Pozzili P, Pitocco D, Visalli N, Cavall M G, Buzzetti R, Crino A, Spera S, Suraci C, Multari G, Cervoni M (2000) No effect of oral insulin on residual beta-cell function in recent-onset type I diabetes (the IMDIAB VII). Diabetologia 43: 1000-1004.
Pugliese A, Brown D, Garza D, Murchison D, Zeller M, Redondo M, Diez J, Eisenbarth G S, Patel D D, Ricordi C (2001) Self-antigen presenting cells expressing diabetes-associated autoantigens exist in both thymus and peripheral lymphoid organs. J Clin Invest 107: 555-564.
Quinn A, McInerney M F, Sercarz E E (2001) MHC Class I-restricted determinants on the glutamic acid decarboxylase 65 molecule induce spontaneous CTL activity. J Immunol 167: 1748 1757.
Raz I, Elias D, Avron A, Tamir M, Metzger M, Cohen I R (2001) P-cell function in new-onset type 1 diabetes and immunomodulation with a heat-shock protein peptide (DiaPep 277): a randomized, double-blind phase II trail. Lancet 358: 1749-1753.
Reijonen H, Novak E J, Kochik S, Henninger A, Liu A W, Kwok W W, Nepom G T (2002) Detection of GAD65-specific T cells by major histocompatibility complex class II tetramers in type I diabetic patients and at-risk subjects. Diabetes 51: 1375-1382.
Roep B O, Arden S D, de Vries R R P, Hutton J C (1990) T cell clones from a type-I diabetes patient respond to insulin secretory granule proteins. Nature 345:632-634.
Rohane P W, Shimada A, Kim D T, Edwards C T, Charlton B, Shultz L D, Fathman C G (1995) Islet-infiltrating lymphocytes from prediabetic NOD mice rapidly transfer diabetes to NOD-scid/scid mice. Diabetes 44: 550-554.
Roncarolo M G, Levings M K, Traversi C (2001) Differentiation of T regulatory cells by immature dendritic cells. J Exp Med 193:F5-F9.
Rubinstein D, Roska A K, Lipsky P E (1986) Liver sinusoidal lining cells express class II major histocompatibility antigens but are poor stimulators if fresh allogenic lymphocytes. J Immunol 137: 1803-3818.
Sempe P, Richard M F, Bach J F, Boitard C (1994) Evidence of CD4+ regulatory T cells in the non-obese diabetic male mouse. Diabetologica 37:337-343.
Simmons, G., J. D. Reeves, C. C. Grogan, L. H. Vandenberghe, F. Baribaud, J. C. Whitbeck, E. Burke, M. J. Buchmeier, E. J. Soilleux, J. L. Riley, R. W. Doms, P. Bates, and S. Pohlmann. 2003. DC-SIGN and DC-SIGNR bind ebola glycoproteins and enhance infection of macrophages and endothelial cells. Virology 305:115.
Skyler J, Rabinovitch A (1992) Effects of cyclosporine in recent onset type I diabetes mellitus. Effects on islet beta cell function. Miami Cyclosporine Diabetes Study Group. J Diabetes 6: 77-88.
Smith J A, Tang Q, Bluestone J A (1 998) Partial TCR signals delivered by Fc-R-nonbinding anti-CD3 monoclonal antibodies differentially regulate individual Th subsets. J Immunol 160:4841-4849.
Smith J A, Tso J Y, Clark M R, Cole M S, Bluestone J A (1997) Nonmitogenic anti-CD3 antibodies deliver a partial T cell receptor signal and induce clonal anergy. J Exp Med 185: 1413-1422.
Soilleux E J, Batten R, Trowsdale J (2000) Cutting Edge: DC-SIGN; a related gene, DC-SIGNR; and CD23 form a cluster on 19˜13. J Immunol 165: 2937-2942.
Thornton A M, Shevach E M (1998) CD4+CD25+ immunoregulatory T cells suppress polyclonal T cell activation in vitro by inhibiting interleukin 2 production. J Exp Med 188: 287-296.
Thorton A M, Shevach E M (2000) Suppressor function of CD4+CD25+ immunoregulatory T cells is antigen nonspecific. J Immunol 164: 183-190.
Tian J D, Clare-Salzler M, Herschenfeld A, Middleton B, Newman D, Mueller R, Arita S, Evans C, Atkinson M A, Mullen Y, Sarvetnick N, Tobin A J, Lehmann P V, Kaufman D L (1996) Modulating autoimmune responses to GAD inhibits disease progression and prolongs islet graft survival in diabetes-prone mice. Nat Med 2: 1348-1353.
Tian J, Atkinson M A, Clare-Salzer M, Herschenfeld A, Forsthuber T, Lehmann P V, Kaufman D L (1996) Nasal administration of glutamate decarboxylase (GAD65) peptides induces Th2 responses and prevents murine insulin-dependent diabetes. J Exp Med 183: 1561-1567.
Tisch R., Yang X D, Singer S M, Liblau R, Fugger L, McDevitt H (1993) Immune response to glutamic acid decarboxylase correlates with insulitis in non-obese diabetic mice. Nature 366: 72-75
Tisch R, Wang B, Atkinson M A, Serreze D V, Friedline R (2001) A glutamic acid decarboxylase 65-specific Th2 cell clone immunoregulates autoimmune diabetes in nonobese diabetic mice. J Immunol 166: 6925-2936.
Tisch R, Wang B, Serreze D V (1999) Induction of glutamic acid decarboxylase 65-specific Th2 cells and suppression of autoimmune diabetes at late stages of disease is epitope dependent. J Immunol 163: 1178-1187.
Van Liempt, E., A. Imberty, C. M. Bank, S. J. Van Vliet, Y. Van Kooyk, T. B. Geijtenbeek, and I. Van Die. 2004. Molecular basis of the differences in binding properties of the highly related C-type lectins DC-SIGN and L-SIGN to Lewis X trisaccharide and Schistosoma mansoni egg antigens. J Biol Chem 279:33161.
Wakkach A, Cottrez F, Groux H (2001) Differentiation of regulatory T cells is induced by CD2 co-stimulation. J Immunol 167:3107-3113.
Weiner H L (2001) Oral tolerance: immune mechanisms and the generation of Th3-type TGF-beta secreting regulatory T cells. Microbes Infect 3: 947-954.
Wen L, Wong F S, Burkly L, Altieri M, Mamalaki C, Kioussis D, Flavell R A, Sherwin R S (1998) Induction of insulitis by glutamic acid decarboxylase peptide-specific and CD4(+) T cells from human DQ transgenic mice. J Clin Invest 102: 947-957.
Wong F S, Visintin I, Wen L, Flavell R A, Janeway C A Jr (1996) CD8 T cell clones from young nonobese diabetic (NOD) islets can transfer rapid onset diabetes in NOD mice in the absence of CD4 cells. J Exp Med 183: 67-76.
Yagi H, Matsumoto M, Kunimoto K, Kawaguchi J, Makino S, Harada M (1992) Analysis of parts of CD4+ and CD8+ T cells in autoimmune diabetes of NOD mice using transfer to NOD athymic mice. Eur J Immunol 22: 2387-2393.
Yamigawa S, Gray J D, Hashimoto S, Horwitz D A (2001) A role for TGF-beta in the generation and expansion of CD4+CD25+ regulatory T cells from human peripheral blood. J Immunol 166: 7282-7289.
Yang R, Liu Q, Grosfeld J L, Pescovitz M D (1994) Intestinal venous drainage through the liver is a prerequisite for oral tolerance induction. J Pediatr Surg 29: 1145-1148.
Zechel M A, Elliott J F, Atkinson M A, Singh B (1998) Characterization of novel T-cell epitopes on 65 kDa and 67 kDa glutamic acid decarboxylase relevant in autoimmune responses in NOD mice. J Autoimmun 1: 83-95.
Zekzer D, Wong S F, Ayalon O, Millet I, Altieri M, Shigeki S, Solimena M, Sherwin R (1998) GAD-reactive CD4+ThI cells induce diabetes in NOD/SCID mice. J Clin. Invest 101: 68-73.
Zhang Z J, Davidson L, Eisenbarth G, Weiner H L (1991) Suppression of diabetes in nonobese diabetic mice by oral administration of porcine insulin. Proc Natl Acad Sci USA 88:10252-10256.

The above description should not be construed as limiting, but merely as exemplifications of preferred embodiments. It will be understood that various modifications may be made to the embodiments disclosed herein. For example, as those skilled in the art will appreciate, the specific sequences described herein can be altered slightly without necessarily adversely affecting the functionality of the antibody or antibody fragment. For instance, substitutions of single or multiple amino acids in the antibody sequence can frequently be made without destroying the functionality of the antibody or fragment. Thus, it should be understood that antibodies having a degree of homology greater than 70% to the specific antibodies described herein are within the scope of this disclosure. In particularly useful embodiments, antibodies having a homology greater than about 80% to the specific antibodies described herein are contemplated. In other useful embodiments, antibodies having a homology greater than about 90% to the specific antibodies described herein are contemplated. Therefore, the above description should not be construed as limiting, but merely as exemplifications of preferred embodiments. Those skilled in the art will envision other modifications within the scope and spirit of this disclosure.

What is claimed is:

1. An antibody that recognizes an L-SIGN receptor and exhibits at least forty-percent internalization into isolated human liver non-parenchymal cells in two hours.

2. An antibody that is internalized into isolated human liver non-parenchymal cells and blocks the binding of ICAM-3 to L-SIGN receptor without affecting the binding of ICAM-1.

3. A composition comprising an antibody in accordance with claim 1 and a pharmaceutically acceptable carrier.

4. A vaccine comprising an antibody in accordance with claim 1.

5. An antibody that recognizes an L-SIGN receptor on a cell, the antibody being capable of effectively blocking binding of a virus to the cell.

6. An antibody in accordance with claim 5, wherein the antibody is capable of effectively blocking binding of a virus selected from the group consisting of HIV, HCV, Ebola, SARS, CMV and Sindbis to the cell.

7. An antibody that recognizes an L-SIGN receptor on a cell, the antibody being capable of effectively blocking infection of the cell by a virus.

8. An antibody in accordance with claim 7, wherein the antibody is capable of effectively blocking infection of the cell by a virus selected from the group consisting of HIV, HCV, Ebola, SARS, CMV and Sindbis.

9. An antibody that recognizes an L-SIGN receptor on a cell, the antibody being capable of effectively blocking transmission of a virus from the cell to another cell.

10. An antibody in accordance with claim 9, wherein the antibody is capable of effectively blocking transmission of a virus selected from the group consisting of HIV, HCV, Ebola, SARS, CMV and Sindbis from the cell to another cell.

11. A vaccine comprising an antibody in accordance with claim 5.

12. An antibody that recognizes an L-SIGN receptor on a cell, the antibody being capable of effectively blocking binding of a bacteria of the genus Mycobacterium to the cell.

13. An antibody in accordance with claim 12, wherein the antibody is capable of effectively blocking binding of a bacteria selected from the group consisting of M. tuberculosis and M. Bovis to the cell.

14. An antibody that recognizes an L-SIGN receptor on a cell, the antibody being capable of effectively blocking infection of the cell by a bacteria of the genus Mycobacterium.

15. An antibody in accordance with claim 14, wherein the antibody is capable of effectively blocking the infection of the cell by bacteria selected from the group consisting of M. tuberculosis and M. Bovis.

16. An antibody that recognizes an L-SIGN receptor on a cell, the antibody being capable of effectively blocking transmission of a bacteria of the genus Mycobacterium from the cell to another cell.

17. An antibody in accordance with claim 16, wherein the antibody is capable of effectively blocking the transmission of bacteria selected from the group consisting of M. tuberculosis and M. Bovis from the cell to another cell.

18. An antibody that recognizes an L-SIGN receptor on a cell, the antibody being capable of effectively blocking the binding of Schistosoma mansoni to the cell.

19. An antibody that recognizes an L-SIGN receptor on a cell, the antibody being capable of effectively blocking the infection of the cell by Schistosoma mansoni.

20. An antibody that recognizes an L-SIGN receptor on a cell, the antibody being capable of effectively blocking the transmission of Schistosoma mansoni from the cell to another cell.

21. A diagnostic agent for a tumor characterized by increased L-SIGN expression comprising an antibody that recognizes an L-SIGN receptor.

22. A diagnostic kit comprising the diagnostic agent of claim 21.

23. A method for diagnosing cancer comprising:
obtaining a tissue sample from a subject suspected of having cancer; and
determining the degree to which the tissue sample binds with an antibody that recognizes an L-SIGN receptor,
wherein an increase in the degree of binding compared to corresponding normal tissue indicates the presence of cancer.

24. A method as in claim 23 wherein the determining step comprises staining for the presence of L-SIGN.

25. A therapeutic agent for treating a cancer characterized by increased L-SIGN expression comprising an antibody that recognizes an L-SIGN receptor.

26. A method for treating a cancer comprising administering to a subject a cancer cell killing amount of a composition comprising an antibody that recognizes an L-SIGN receptor.

27. The method of claim 26 wherein the antibody that recognizes the L-SIGN receptor induces antibody-dependent cellular cytotoxicity of cancer cells.

28. The method of claim 26 wherein the antibody that recognizes the L-SIGN receptor induces complement-dependent cytotoxicity of cancer cells.

29. The method of claim 26 wherein the antibody that recognizes the L-SIGN receptor prevents negative regulation of the immune system through L-SIGN expressing cancer cells.

30. A method as in claim 26 wherein the antibody that recognizes the L-SIGN receptor is fused to a toxin.

31. A method as in claim 24 wherein the antibody that recognizes the L-SIGN receptor is fused to a high energy radiation emitter.

32. A method for treating an inflammatory disease comprising administering to a subject an L-SIGN expressing cell killing amount of a composition comprising an antibody that recognizes an L-SIGN receptor.

33. The method of claim 32 wherein the antibody that recognizes the L-SIGN receptor induces antibody-dependent cellular cytotoxicity of L-SIGN expressing cells.

34. The method of claim 32 wherein the antibody that recognizes the L-SIGN receptor induces complement-dependent cytotoxicity of L-SIGN expressing cells.

35. The method of claim 32 wherein the antibody that recognizes the L-SIGN receptor prevents negative regulation of the immune system through L-SIGN expressing cells.

36. A method as in claim 32 wherein the antibody that recognizes the L-SIGN receptor is fused to a toxin.

37. A method as in claim 32 wherein the antibody that recognizes the L-SIGN receptor is fused to a high energy radiation emitter.

38. An antibody that possesses high affinity for L-SIGN but low affinity for DC-SIGN.

39. An antibody that binds linear epitopes on L-SIGN and conformational epitopes on L-SIGN.

40. An antibody that blocks L-SIGN-T-cell interaction thereby causing proliferation of regulatory T-cells.

41. An antibody in accordance with claim 40, wherein the proliferation of regulatory T-cells occurs in the liver.

42. An antibody that blocks L-SIGN-T-cell interaction thereby suppressing proliferation of auto-reactive T-cells.

43. An antibody in accordance with claim 42, wherein the suppression of the proliferation of auto-reactive T-cells occurs in the lymph nodes.

44. An antibody-peptide construct that delivers an auto-antigen to liver sinusoidal endothelial cells thereby stimulating proliferation of regulatory cells.

45. An antibody-peptide construct that delivers an auto-antigen to liver sinusoidal endothelial cells thereby suppressing activity of auto-reactive T-cells.

46. An antibody-peptide construct that delivers a vaccine antigen to sinusoidal endothelial cells in lymph nodes thereby stimulating proliferation of antigen specific T-cells.

47. A method for isolating L-SIGN expressing cells comprising:
obtaining a mixture of L-SIGN expressing cells in combination with cells not expressing L-SIGN;
exposing the mixture to anti-L-SIGN antibody; and
allowing the L-SIGN expressing cells to bind to the anti-L-SIGN antibody thereby isolating the L-SIGN expressing cells from the cells not expressing L-SIGN.

48. A composition comprising an antibody in accordance with claim 2 and a pharmaceutically acceptable carrier.

49. A vaccine comprising an antibody in accordance with claim 2.

50. A vaccine comprising an antibody in accordance with claim 7.

51. A vaccine comprising an antibody in accordance with claim 9.
